(19) United States
(12) Patent Application Publication
(10) Pub. No.: US 2021/0380586 A1
(43) Pub. Date: Dec. 9, 2021

(54) 2-INDOLYL IMIDAZO[4,5-D]PHENANTHROLINE POLYMORPHS AND COMPOSITIONS REGARDING THE SAME

(71) Applicant: Aptose Biosciences Inc., Mississauga (CA)

(72) Inventor: William G. RICE, Del Mar, CA (US)

(21) Appl. No.: 17/336,823

(22) Filed: Jun. 2, 2021

Related U.S. Application Data
(60) Provisional application No. 63/033,343, filed on Jun. 2, 2020.

(51) Int. Cl. C07D 471/14; A61K 47/14; A61K 47/10

(52) U.S. Cl. CPC C07D 471/14; C07B 2200/13; A61K 47/10; A61K 47/14

(57) ABSTRACT

The invention relates to solid forms of 2-indolyl imidazo[4,5-D]phenanthroline, methods of their preparation, pharmaceutical compositions thereof and methods of their use.

### CROSS-REFERENCE TO RELATED APPLICATIONS

This application claims the benefit of U.S. Provisional Application No. 63/033,343, filed Jun. 2, 2020, the disclosure of which is incorporated by reference herein in its entirety.

### FIELD OF THE DISCLOSURE

The present disclosure relates to solid forms of 2-indolyl imidazo[4,5-D]phenanthroline, processes for preparing such solid forms, pharmaceutical compositions thereof, and method of treating cancer using the same.

### BACKGROUND OF THE DISCLOSURE

Metal chelators have been developed for the treatment of diseases resulting from metal overload. For example, iron chelators, such as desferrioxamine (DFO), have been studied as potential anticancer therapies, as iron has an important role in active sites of a wide range of proteins involved in energy metabolism, respiration, and DNA synthesis. See also U.S. Pat. No. 6,589,966 and U.S. Patent Application No. 2002/0119955. Further, there has been interest in zinc chelators as a potential anti-cancer agent (Zhao, R., et al. (2004) Biochem Pharmacol 67(9): 1677-88). Alternatively, other metal chelators may exert anti-neoplastic effects through the formation of cytotoxic chelate complexes, for example with redox-active metals, iron and copper.

1,10-Phenanthroline (OP) is a well-known metal chelator. Studies have investigated derivatives of 1,10-phenanthroline and their ability to chelate various metals. For example, Chao et al., have synthesized 1,3-bis([1,10]) phenanthroline-[5,6-d]imidazol-2-yl)benzene (mbpibH2) and its (bpy)2Ru²⁺ complexes and studied their electrochemical and spectroscopic properties (Polyhedron, 2000, 1975-1983). Liu et al., prepared ruthenium complexes with 2-(2-hydroxyphenyl)imidazo[4,5-f][1,10]phenanthroline (HPIP) and studied the binding behaviour of these complexes towards calf thymus DNA (JBIC, 2000, 5, 119-128). Similarly, Xu et al., have described the synthesis of 2-(4-methylphenyl)imidazol[4,5-f]1,10-phenanthroline and its Ru(II) complexes and binding of the prepared complexes to calf thymus DNA (New J. Chem., 2003, 27, 1255-1263).

More recently, International Patent Application Nos. PCT/CA2003/001229, PCT/IB2004/052433, PCT/IB2006/051675, and PCT/US2014/031349 describes a broad class of 2,4,5-trisubstituted imidazole compounds, including 2-substituted imidazo[4,5-D]phenanthroline derivatives, and their use in the treatment of cancer, which are hereby incorporated by reference in their entireties.

### SUMMARY OF THE DISCLOSURE

This disclosure relates to a crystalline form of Compound I free base tetrahydrate.

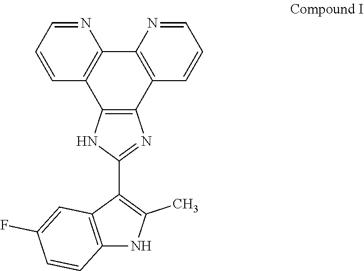 [SMILES: Cc1[nH]c2ccc(F)cc2c1-c1nc2c3cccnc3c3ncccc3c2[nH]1]

In one embodiment, the crystalline form of the present disclosure is substantially pure. In one embodiment, the crystalline form of Compound I free base tetrahydrate has a chemical purity of greater than about 95%. In one embodiment, the crystalline form of Compound I free base tetrahydrate has a chemical purity of greater than about 98%. In one embodiment, the crystalline form of Compound I free base tetrahydrate has a chemical purity of greater than about 99%.

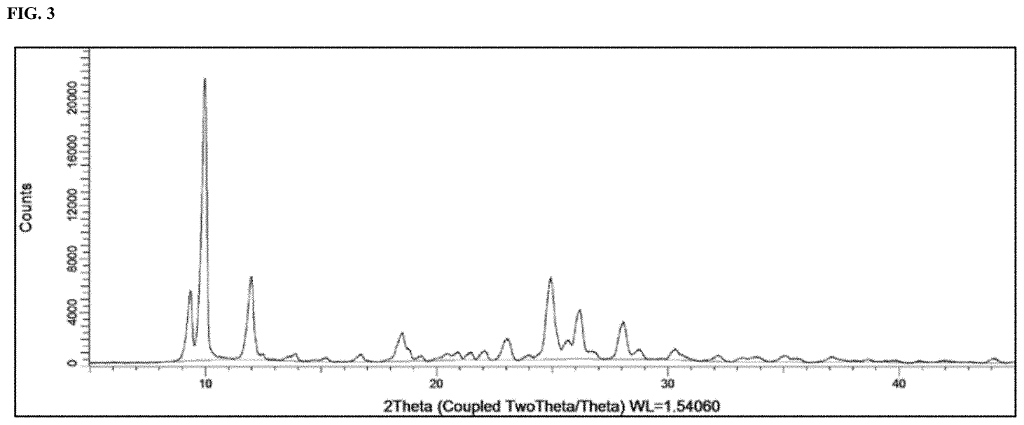

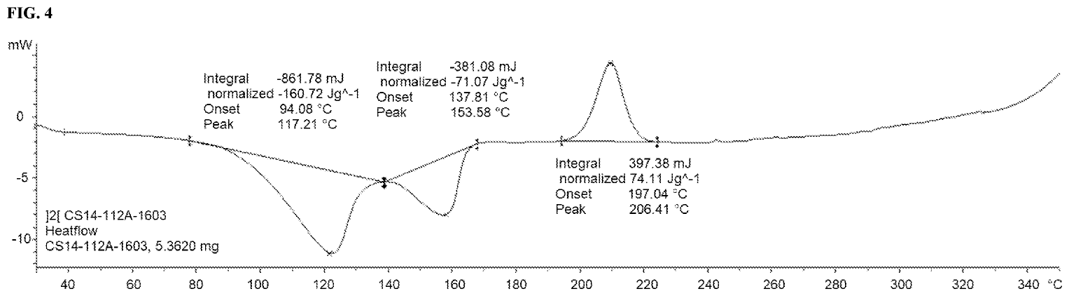

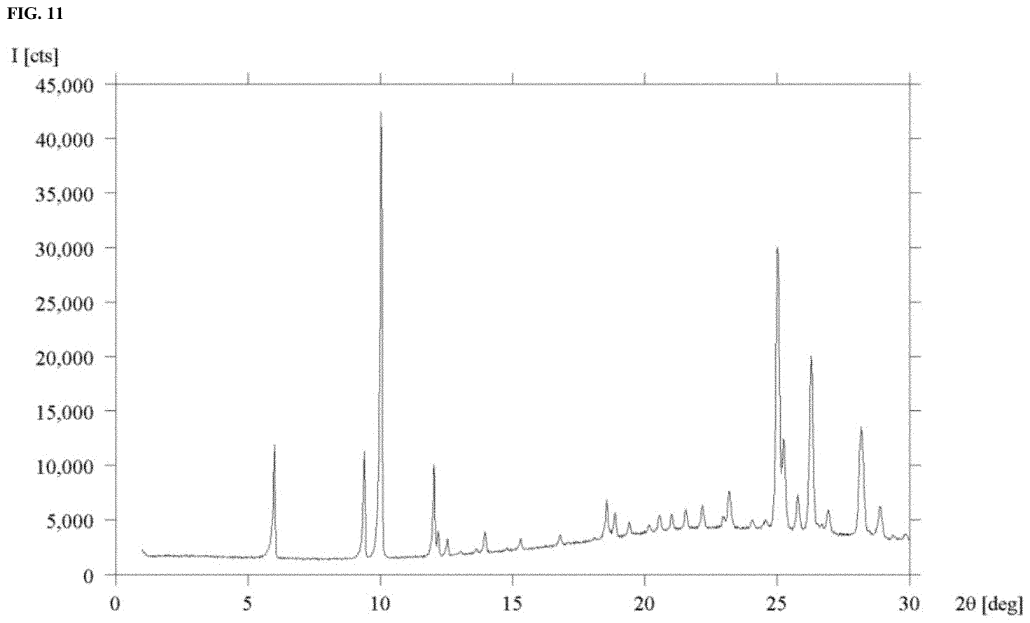

In one embodiment, the crystalline form of the present disclosure exhibits an X-ray powder diffraction (XRPD) pattern substantially similar to FIG. 11. In one embodiment, the crystalline form of the present disclosure exhibits an X-ray powder diffraction (XRPD) pattern substantially similar to FIG. 3. In one embodiment, the crystalline form of the present disclosure exhibits an XRPD pattern comprising peaks at 10.0±0.2 and at 25.0±0.2 degrees two-theta. In some embodiments, the crystalline form of the present disclosure exhibits an XRPD pattern comprising peaks at 26.3±0.2 and 28.2±0.2 degrees two-theta. In some embodiments, the crystalline form of the present disclosure exhibits an XRPD pattern comprising peaks at 6.0±0.2, 9.4±0.2 and 25.2±0.2 degrees two-theta.

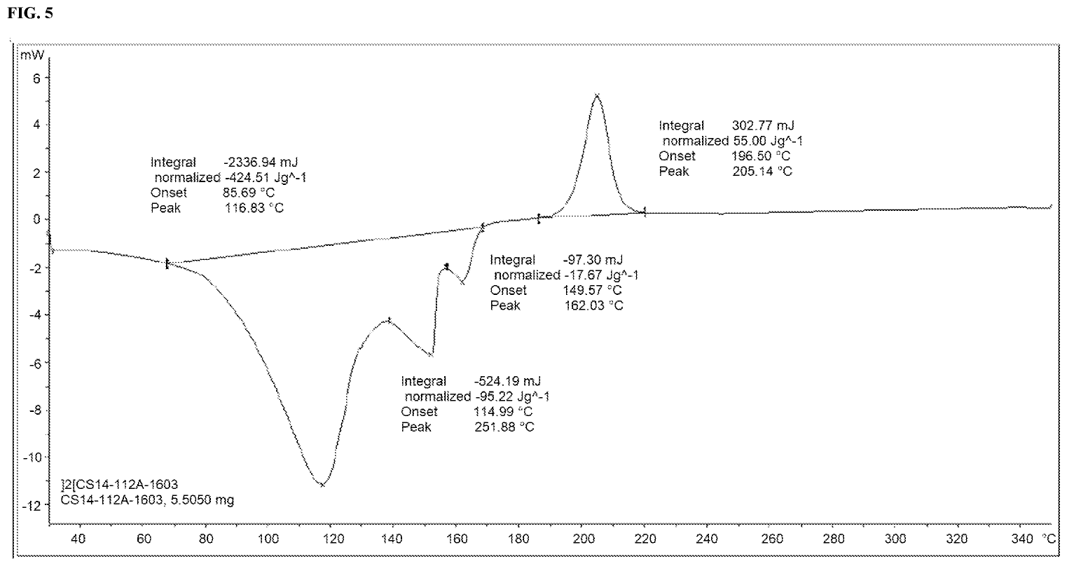

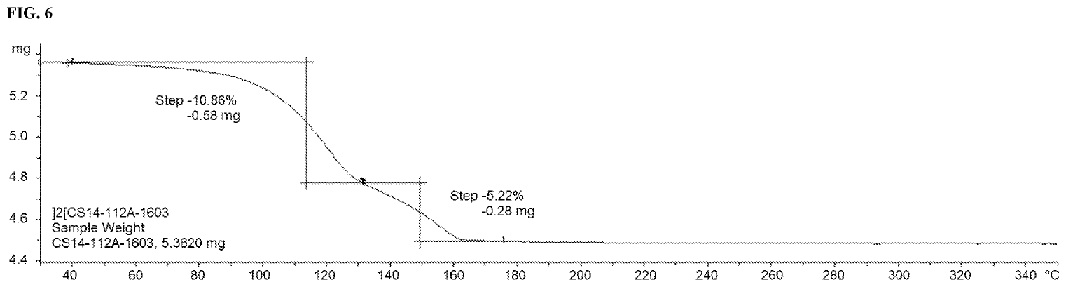

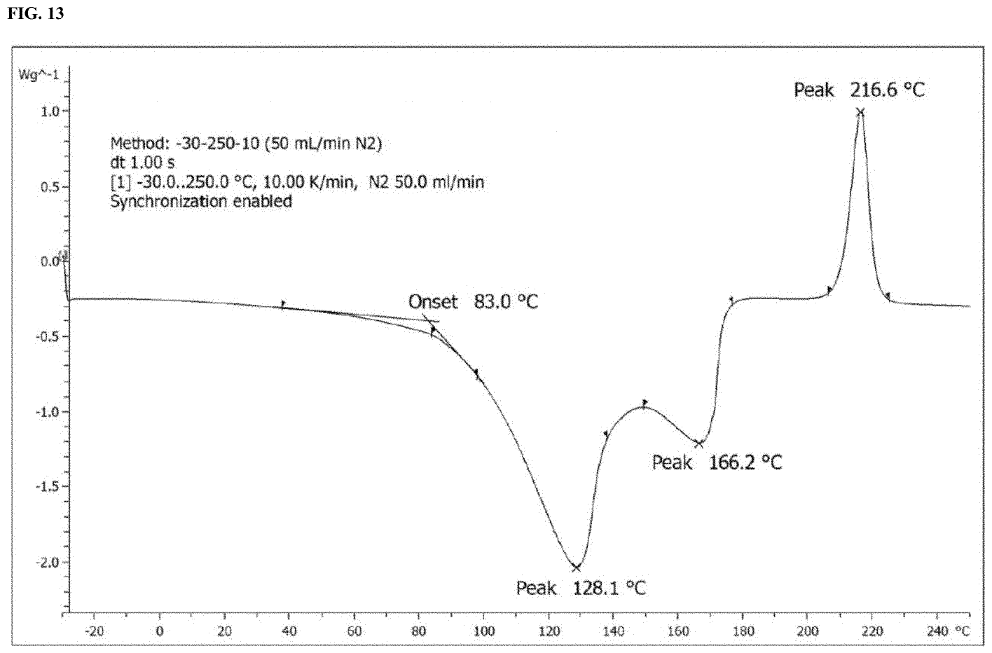

In one embodiment, the crystalline form of the present disclosure exhibits a DSC (differential scanning calorimetry) thermogram substantially similar to FIG. 4, FIG. 5, or FIG. 13. In one embodiment, the crystalline form of the present disclosure exhibits a DSC thermogram comprising an exotherm peak (max) between about 200° C. to about 220° C. In one embodiment, the crystalline form of the present disclosure exhibits a DSC thermogram comprising an exotherm peak (max) between 205° C.±0.5° C. to about 207° C.±0.5° C. In some embodiments, the crystalline form of the present disclosure exhibits a DSC thermogram further comprises at least two endotherm peaks between about 60° C. to about 180° C. In some embodiments, the crystalline form of the present disclosure exhibits a DSC thermogram further comprises an endotherm peak (max) between about 105° C. to about 130° C. In some embodiments, the crystalline form of the present disclosure exhibits a DSC thermogram further comprises an endotherm peak (max) between about 140° C. to about 170° C.

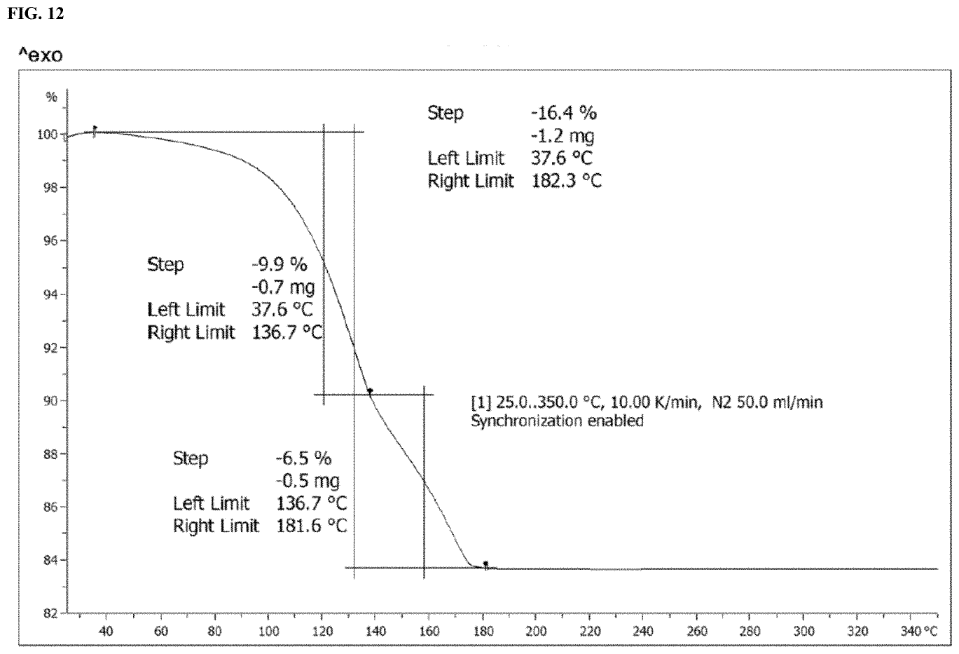

In one embodiment, the crystalline form of the present disclosure exhibits a TGA (thermogravimetric analysis) thermogram substantially similar to FIG. 6 or FIG. 12.

In one embodiment, the crystalline form of the present disclosure is isolated. In one embodiment, the crystalline form of the present disclosure is purified.

In one embodiment, the present disclosure relates to compositions comprising any one of the crystalline forms disclosed herein. In one embodiment, the composition comprises Compound I free base tetrahydrate.

In one embodiment, the present disclosure relates to pharmaceutical compositions comprising any one of the crystalline forms disclosed herein and a pharmaceutically acceptable carrier or excipient. In one embodiment, the pharmaceutical composition comprises Compound I free base tetrahydrate.

In one embodiment, the pharmaceutical compositions as disclosed herein are substantially free of Compound I acetate solvate and Compound I HCl salt.

In embodiment of the pharmaceutical compositions comprising Compound I free base tetrahydrate Form 2, the composition comprises a crystalline Compound I Form 1, Form 3, Form 4, Form 5, or Form 6 in an amount of less than about 5% by weight. In embodiment of the pharmaceutical compositions comprising Compound I free base tetrahydrate Form 2, the composition comprises a crystalline Compound I Form 1, Form 3, Form 4, Form 5, or Form 6 in an amount of less than about 1% by weight. In embodiment of the pharmaceutical compositions comprising Compound I free base tetrahydrate Form 2, the composition comprises a crystalline Compound I Form 1, Form 3, Form 4, Form 5, or Form 6 in an amount of less than about 0.5% by weight.

In embodiment of the pharmaceutical compositions comprising Compound I free base tetrahydrate Form 2, the composition comprises a crystalline Compound I Form 1, Form 3, Form 4, Form 5, or Form 6 in an amount of about 0.05% to about 50% by weight.

In one embodiment, the present disclosure relates to pharmaceutical compositions comprising Compound I or a salt or a solvate thereof, propylene glycol (PG) and macrogol (15)-hydroxystearate. In one embodiment, the Compound I is Compound I free base tetrahydrate. In one embodiment, the Compound I is crystalline Compound I free base tetrahydrate. In one embodiment, the Compound I is crystalline Compound I Form 2.

In one embodiment, the pharmaceutical compositions as disclosed herein the Compound I is present at a concentration below about 8 mg/mL. In some embodiments, the Compound I is present at a concentration ranging from about 5 mg/mL to about 3 mg/mL.

In one embodiment, the pharmaceutical compositions as disclosed herein are in a form of a solution. In one embodiment, the pharmaceutical compositions as disclosed herein have a water content is below about 12% by volume. In one embodiment, the water content is between about 4% to about 10% by volume.

In one embodiment, the pharmaceutical compositions as disclosed herein, the composition comprises (a) propylene glycol in about 60% to about 80% by volume; (b) macrogol (15)-hydroxystearate in about 15% to about 30% by volume; and (c) water in about 3% to about 12% by volume.

In one embodiment, the pharmaceutical compositions as disclosed herein, the composition comprises (a) propylene glycol in about 70% by volume; (b) macrogol (15)-hydroxystearate in about 23% by volume; and (c) water in about 7% by volume.

In one embodiment, the pharmaceutical compositions as disclosed herein is substantially free of polyethylene glycol.

In one embodiment, the pharmaceutical compositions as disclosed herein is diluted in IV fluid selected from sterile water, dextrose in water, glucose in water, invert sugar in water, saline solution in water (NaCl), sodium bicarbonate solution in water, sodium lactate solution in water, lactated Ringer's solution, or combinations thereof. In some embodiments, the pharmaceutical composition is diluted in IV fluid selected from 5% dextrose in water, 10% dextrose in water, lactated Ringer's solution, saline solution in water, or combinations thereof.

In one embodiment, the pharmaceutical compositions as disclosed herein, the Compound I or a salt or a solvate thereof stays in solution for at least about 120 minutes.

In one embodiment, the pharmaceutical compositions as disclosed herein, the pharmaceutical composition is stable for at least one month when stored at 25° C. in 60% relative humidity.

In one embodiment, the present disclosure relates to a crystalline form of Compound I, or a pharmaceutically acceptable salt, solvate, or hydrate thereof, wherein the crystalline form is selected from Crystalline Form A, Crystalline Form B, Crystalline Form 1, Crystalline Form 2, Crystalline Form 3, Crystalline Form 4, Crystalline Form 5, or Crystalline Form 6.

In one embodiment, the crystalline form of the present disclosure is substantially pure. In one embodiment, the crystalline form of Compound I has a chemical purity of greater than about 95%. In one embodiment, the crystalline form of Compound I is isolated. In one embodiment, the crystalline form of Compound I is purified.

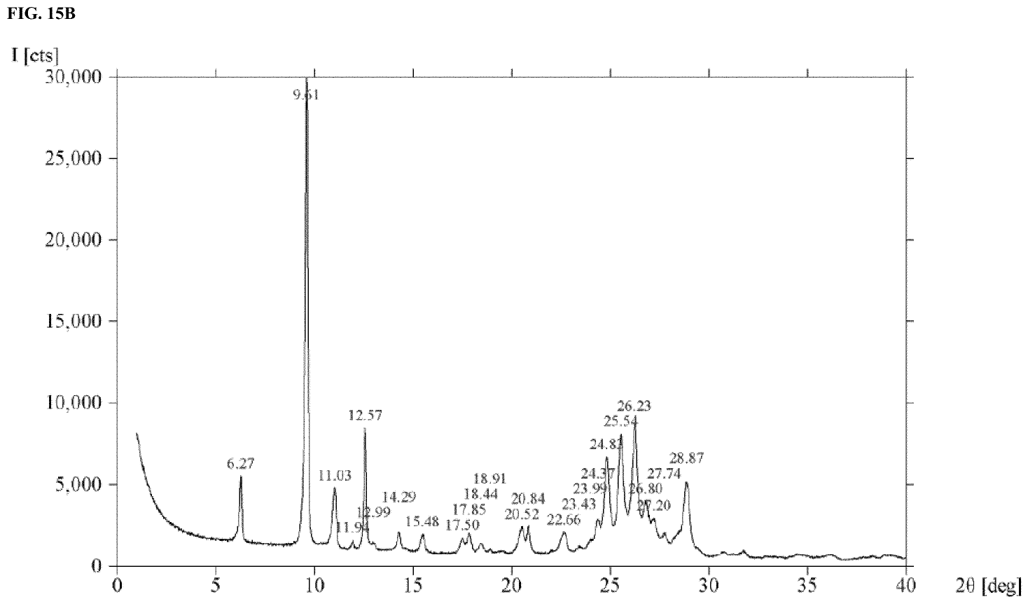

In one embodiment, the crystalline form of Compound I is Form 3. In one embodiment, Crystalline Form 3 exhibits an XRPD pattern comprising peaks at 9.6±0.2, 12.6±0.2 and 26.2±0.2 degrees two-theta. In one embodiment, Form 3 exhibits an XRPD pattern substantially similar to FIG. 15B.

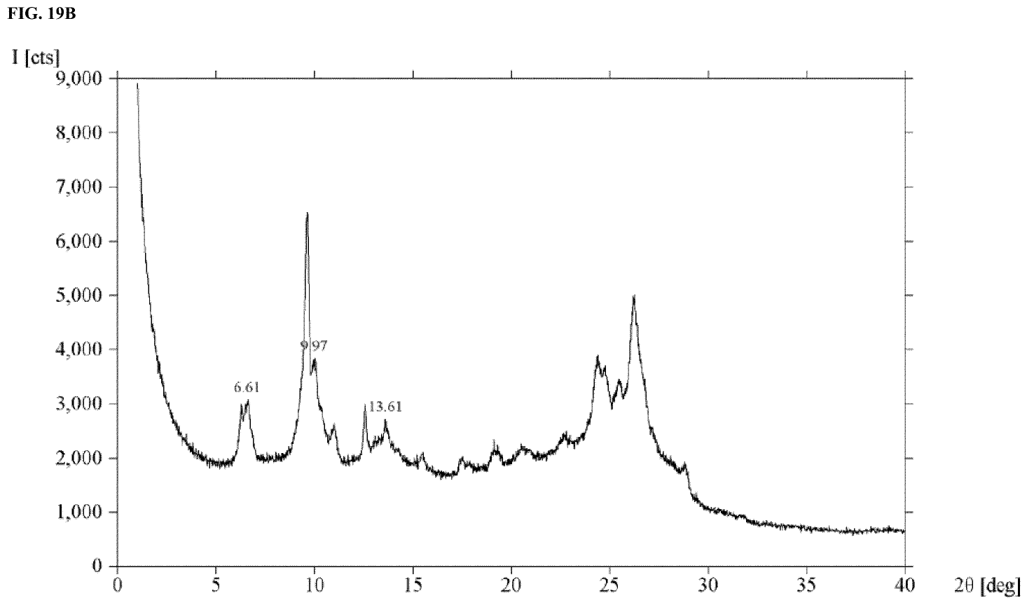

In one embodiment, the crystalline form of Compound I is Form 4. In one embodiment, Crystalline Form 4 exhibits an XRPD pattern comprising peaks at 6.6±0.2, 10.0±0.2 and 13.6±0.2 degrees two-theta. In one embodiment, Form 4 exhibits an XRPD pattern substantially similar to FIG. 19B.

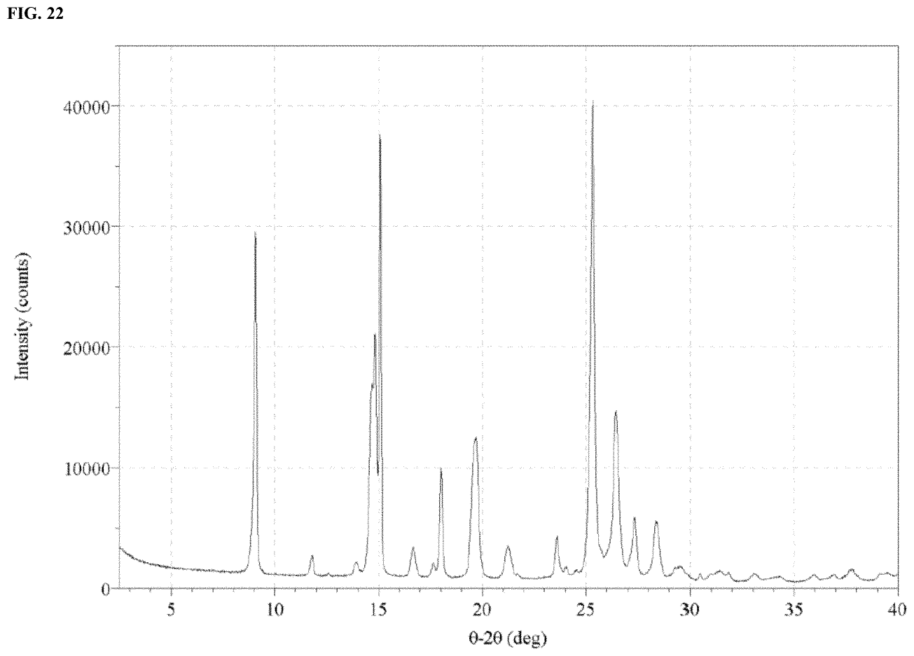

In one embodiment, the crystalline form of Compound I is Form 5. In one embodiment, Crystalline Form 5 exhibits an XRPD pattern comprising peaks at 14.5±0.2 and 21.0±0.2 degrees two-theta. In one embodiment, Form 5 exhibits an XRPD pattern substantially similar to FIG. 22.

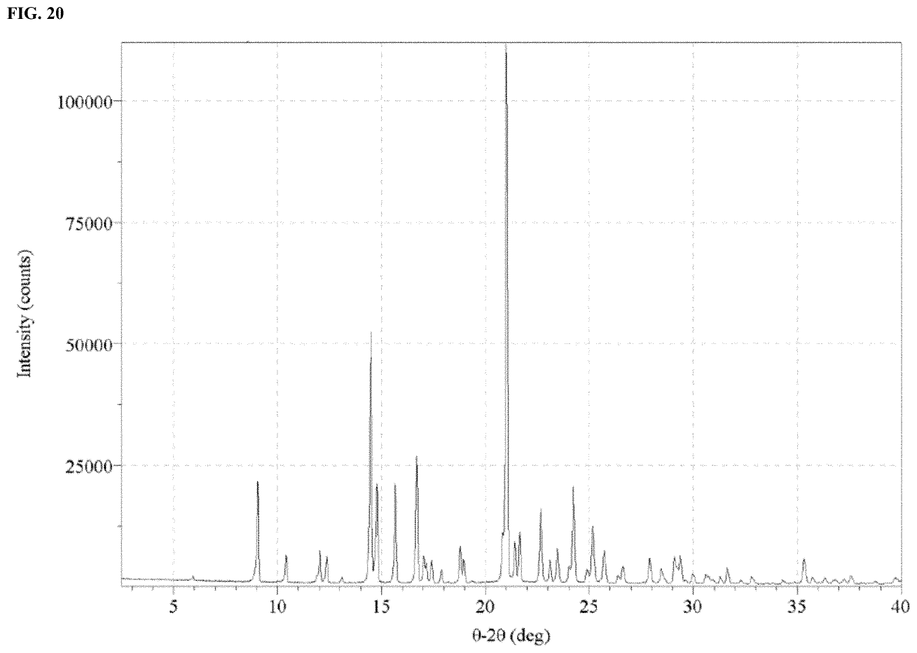

In one embodiment, the crystalline form of Compound I is Form 6. In one embodiment, Crystalline Form 6 exhibits an XRPD pattern comprising peaks at 9.1±0.2, 15.1±0.2, and 25.3±0.2 degrees two-theta. In one embodiment, Form 6 exhibits an XRPD pattern substantially similar to FIG. 20.

In one embodiment, the present disclosure relates to a pharmaceutical composition comprising two or more crystalline form of Compound I, or a pharmaceutically acceptable salt, solvate, or hydrate thereof, selected from Crystalline Form A, Crystalline Form B, Crystalline Form 1, Crystalline Form 2, Crystalline Form 3, Crystalline Form 4, Crystalline Form 5, or Crystalline Form 6.

In one embodiment, the present disclosure relates to methods of treating cancer, comprising administering any one of the crystalline forms of Compound I or a salt or solvate thereof to a subject. In one embodiment, the method comprises administering any one of the crystalline forms of Compound I or a pharmaceutically acceptable salt or solvate thereof to a subject.

In one embodiment, the present disclosure relates to methods of treating cancer, comprising administering any one of the compositions or pharmaceutical compositions comprising Compound I or a salt or solvate thereof to a subject. In one embodiment, the method comprises administering any one of the compositions or pharmaceutical compositions comprising Compound I or a pharmaceutically acceptable salt or solvate thereof to a subject.

In one embodiment of the methods disclosed herein, the cancer is acute myeloid leukemia or myelodysplastic syndrome. In one embodiment of the methods disclosed herein, the cancer is acute myeloid leukemia. In one embodiment, the cancer is relapsed or refractory acute myeloid leukemia or relapsed or refractory myelodysplastic syndrome.

In one embodiment, the present disclosure relates to a kit comprising: a first composition comprising any one of the pharmaceutical compositions disclosed herein comprising a Compound I or a salt or solvate thereof; and a second composition comprising the IV fluid selected from sterile water, dextrose in water, glucose in water, invert sugar in water, saline solution in water (NaCl), sodium bicarbonate solution in water, sodium lactate solution in water, lactated Ringer's solution, or combinations thereof. In one embodiment, the kit comprises a first composition comprising any one of the pharmaceutical compositions disclosed herein comprising a Compound I or a pharmaceutically acceptable salt or solvate thereof; and a second composition comprising the IV fluid selected from sterile water, dextrose in water, glucose in water, invert sugar in water, saline solution in water (NaCl), sodium bicarbonate solution in water, sodium lactate solution in water, lactated Ringer's solution, or combinations thereof.

This disclosure also relates to Compound I free base tetrahydrate. This disclosure also relates to a pharmaceutical composition comprising Compound I free base tetrahydrate. This disclosure also relates to methods of treating cancer comprising administering Compound I free base tetrahydrate to a subject. In one embodiment, cancer is acute myeloid leukemia or myelodysplastic syndrome.

### DETAILED DESCRIPTION OF THE DRAWINGS

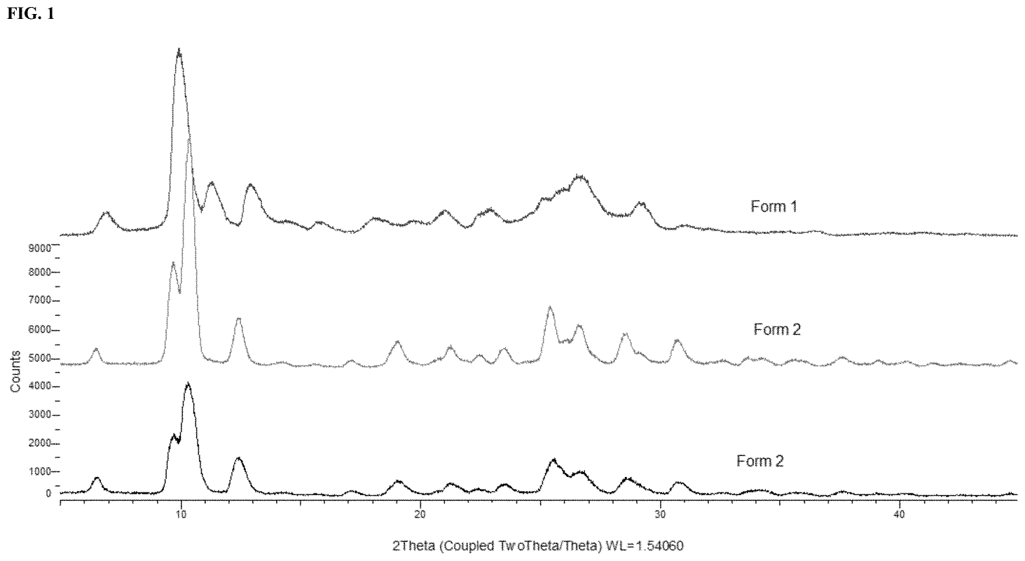

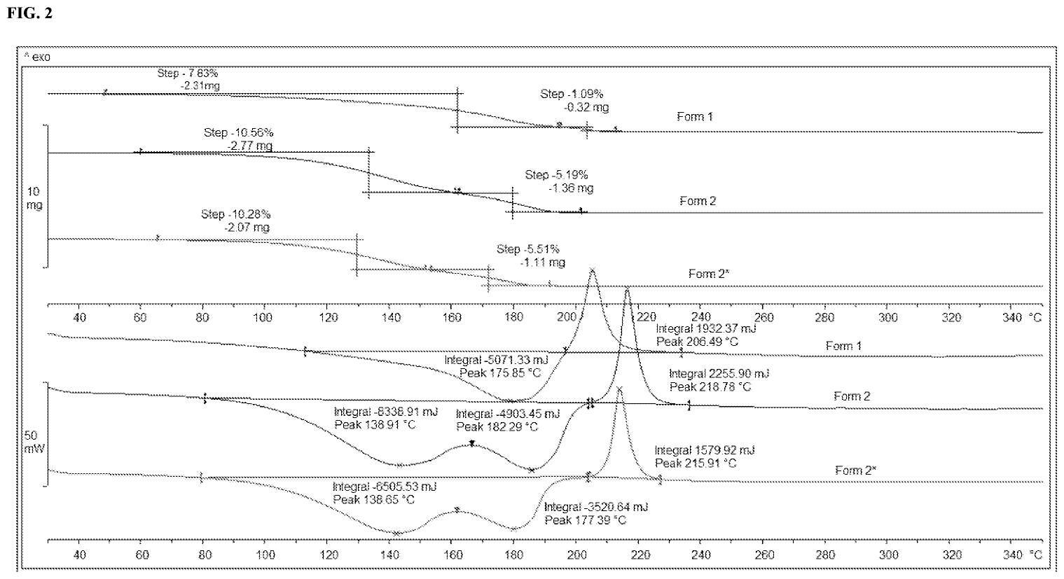

FIG. 1 shows overlay of X-ray powder diffraction (XRPD) of crystalline Compound I-acetate Form 1 and two different samples of crystalline Compound I-tetrahydrate Form 2.

FIG. 2 shows overlay of differential scanning calorimetry (DSC) thermograms and thermogravimetric analysis (TGA) thermograms of crystalline Compound I-acetate Form 1 and two different samples of crystalline Compound I-tetrahydrate Form 2.

FIG. 3 shows X-ray powder diffraction (XRPD) of Crystalline Form 2 of Compound I-hydrate.

FIG. 4 shows differential scanning calorimetry (DSC) thermogram of Crystalline Form 2 of Compound I-hydrate.

FIG. 5 shows differential scanning calorimetry (DSC) thermogram of Crystalline Form 2 of Compound I-hydrate from a different batch than FIG. 4.

FIG. 6 shows thermogravimetric analysis (TGA) thermogram of Crystalline Form 2 of Compound I-hydrate.

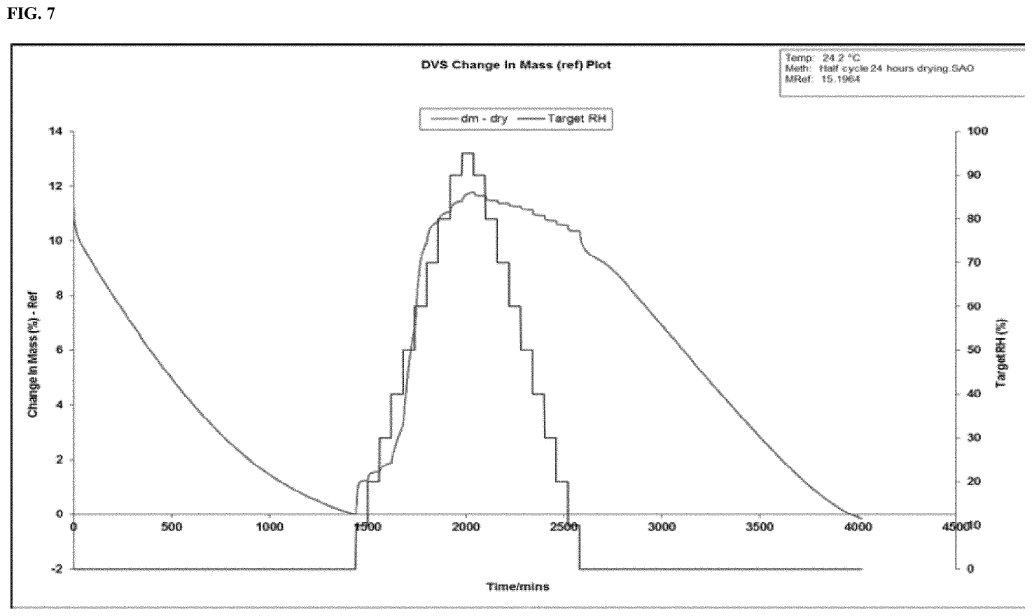

FIG. 7 shows dynamic vapor sorption (DVS) sorption and de-sorption plot of Crystalline Form 2 of Compound I-hydrate.

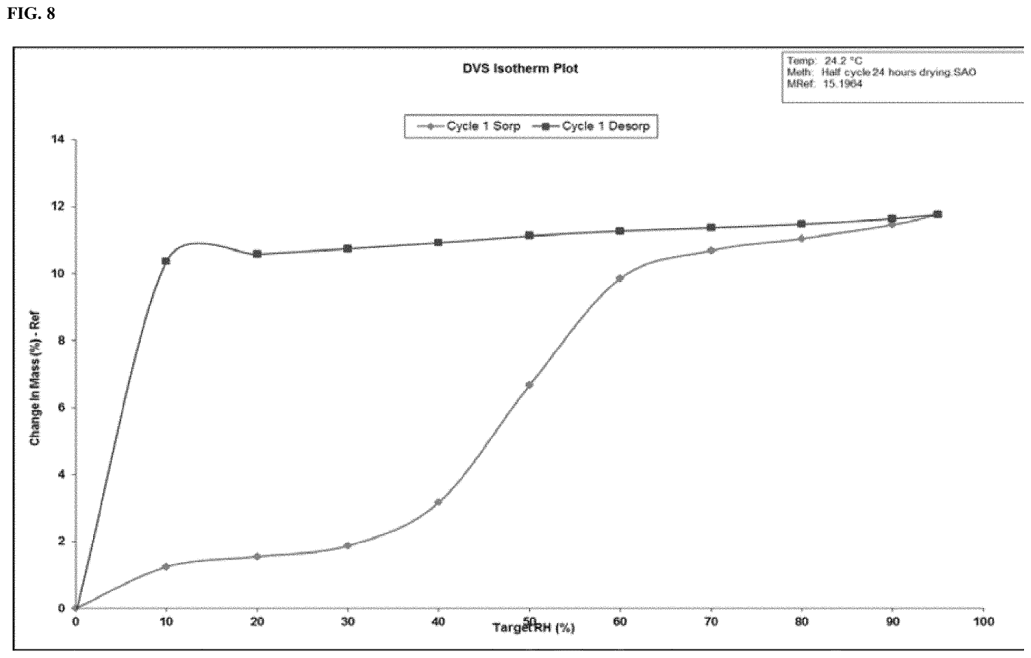

FIG. 8 shows DVS isotherm plot of Crystalline Form 2 of Compound I-hydrate.

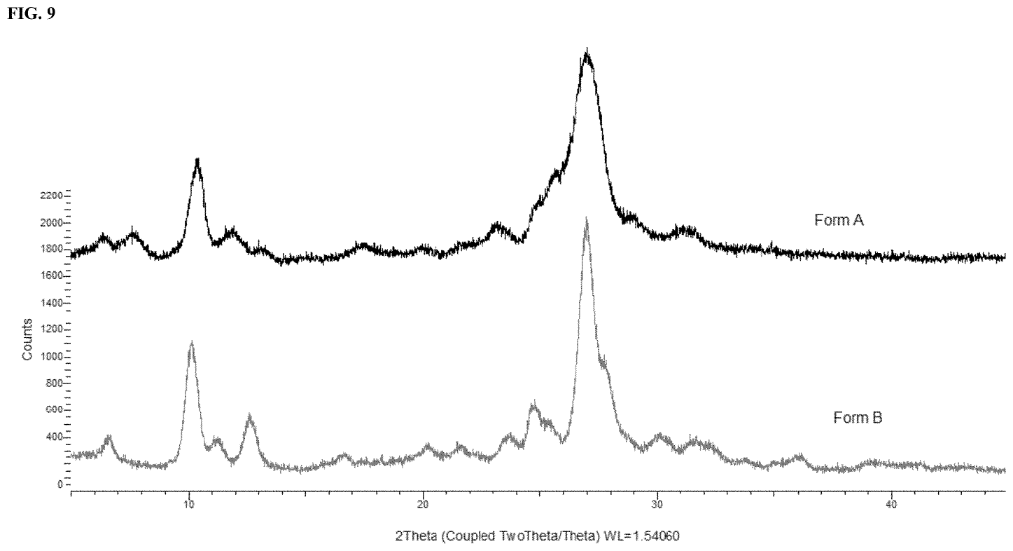

FIG. 9 shows overlay of X-ray powder diffraction (XRPD) patterns of crystalline Compound I-HCl Form A and Form B.

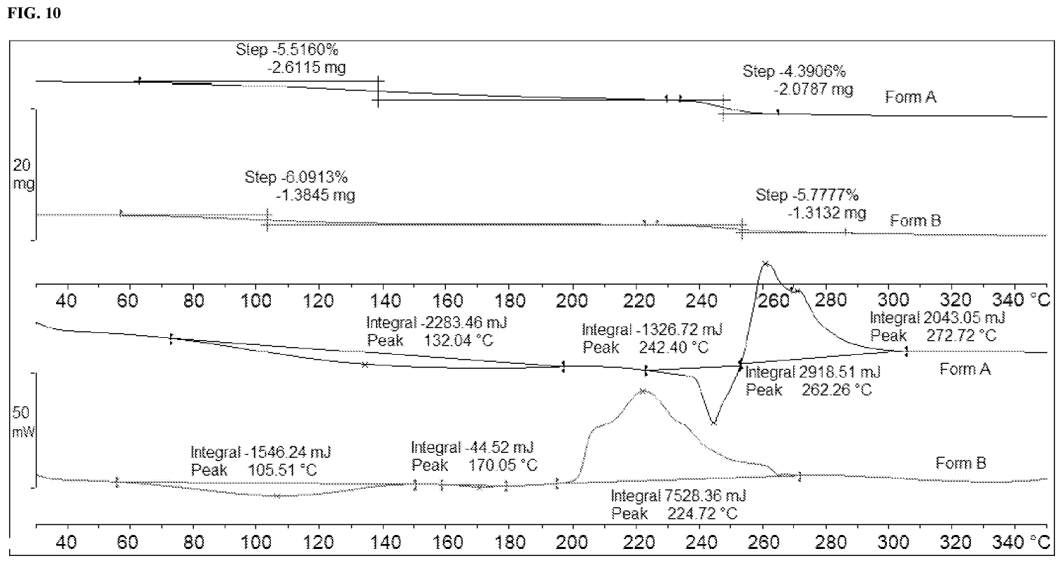

FIG. 10 shows overlay of differential scanning calorimetry (DSC) thermograms and thermogravimetric analysis (TGA) thermograms of crystalline Compound I-HCl Form A and Form B.

FIG. 11 shows X-ray powder diffraction (XRPD) of Crystalline Form 2 of Compound I-hydrate.

FIG. 12 shows thermogravimetric analysis (TGA) thermogram of Crystalline Form 2 of Compound I-hydrate prepared according to Example 10.

FIG. 13 shows differential scanning calorimetry (DSC) thermogram of Crystalline Form 2 of Compound I-hydrate prepared according to Example 10.

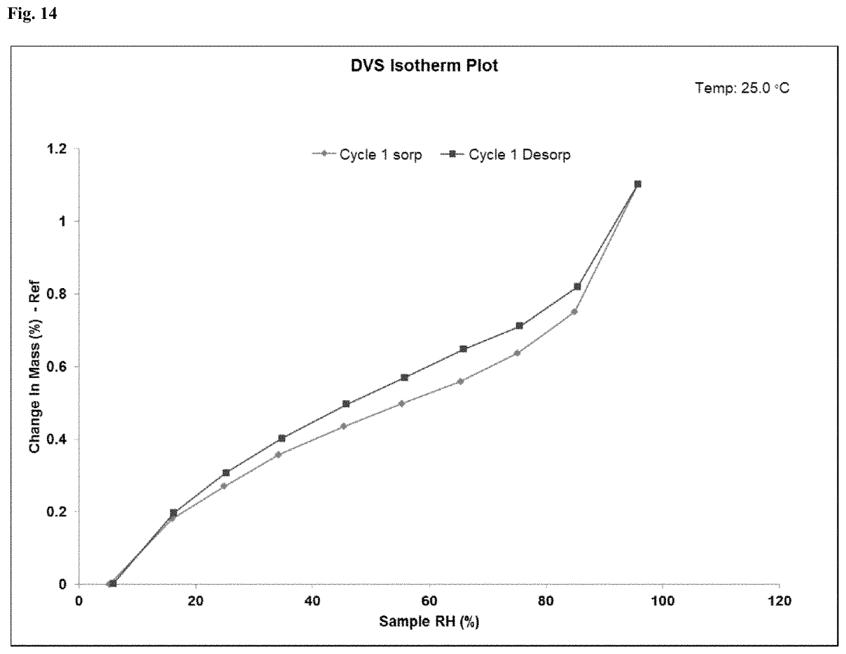

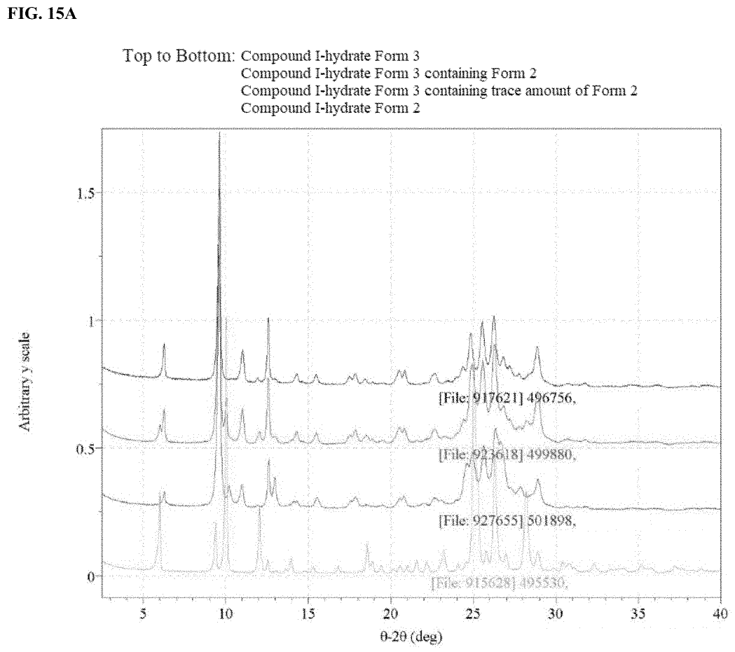

FIG. 14 shows DVS isotherm plot of Crystalline Form 2 of Compound I-hydrate prepared according to Example 10.

FIG. 15A shows overlay of X-ray powder diffraction (XRPD) patterns of crystalline Compound I-hydrate Form 2 and Form 3 as well as a sample containing both forms. FIG. 15B shows of X-ray powder diffraction (XRPD) pattern of crystalline Compound I-hydrate Form 3.

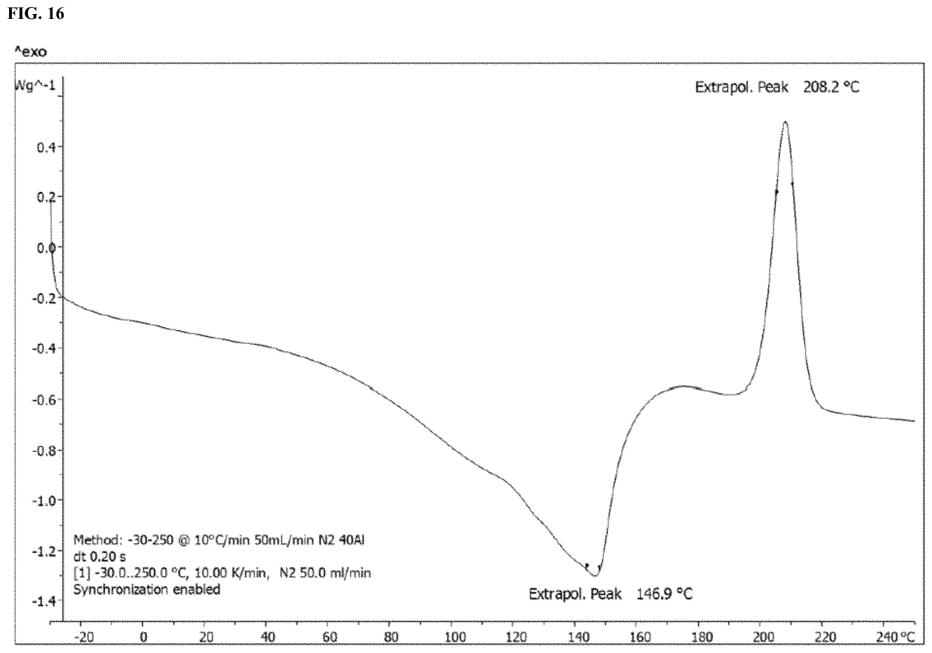

FIG. 16 shows differential scanning calorimetry (DSC) thermogram of Crystalline Form 3 of Compound I-hydrate.

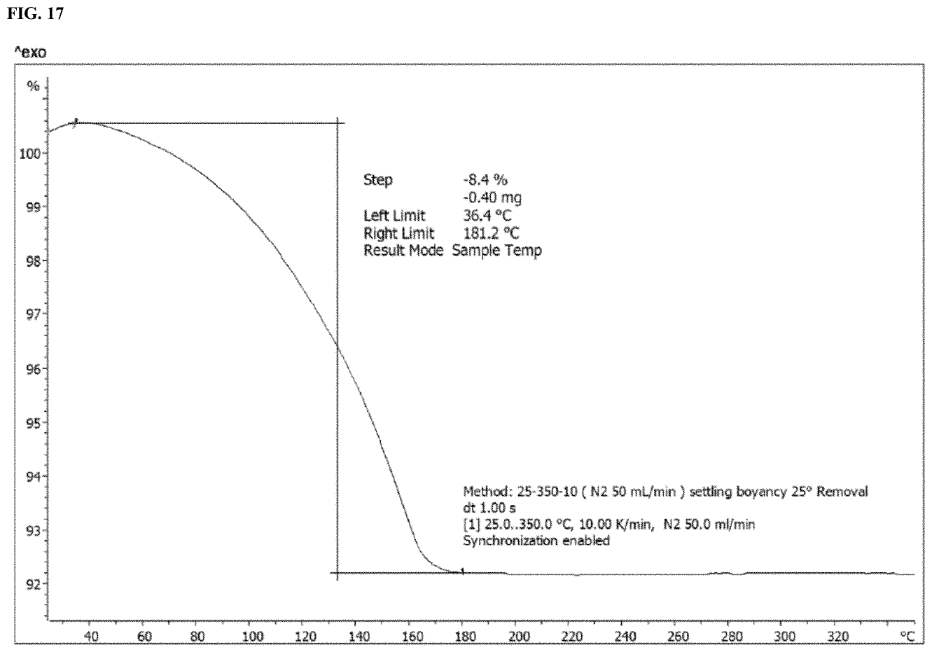

FIG. 17 shows thermogravimetric analysis (TGA) thermogram of Crystalline Form 3 of Compound I-hydrate.

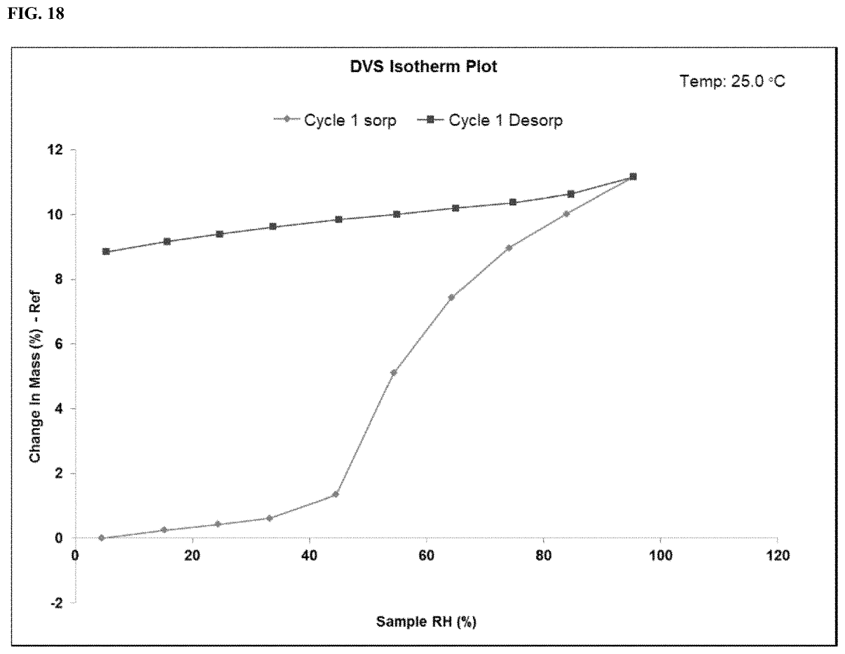

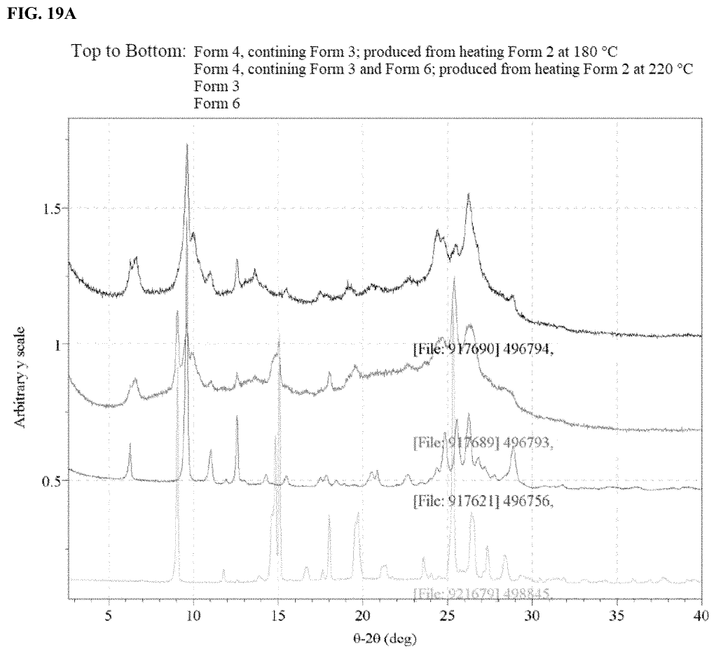

FIG. 18 shows DVS isotherm plot of Crystalline Form 3 of Compound I-hydrate.

FIG. 19A shows overlay of X-ray powder diffraction (XRPD) patterns of crystalline Compound I Form 4 and Form 3 as well as a sample containing both forms. FIG. 19B shows X-ray powder diffraction (XRPD) pattern of crystalline Compound I Form 4.

FIG. 20 shows an X-ray powder diffraction (XRPD) pattern of crystalline Compound I Form 5.

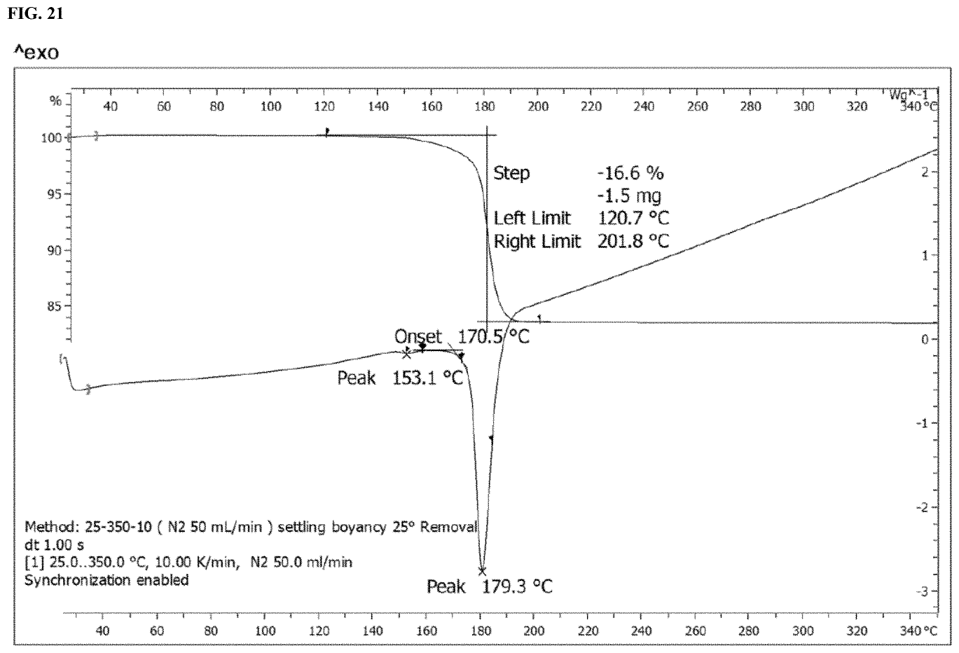

FIG. 21 shows overlay of a differential scanning calorimetry (DSC) thermogram and a thermogravimetric analysis (TGA) thermogram of Compound I Form 5.

FIG. 22 shows an X-ray powder diffraction (XRPD) pattern of crystalline Compound I Form 6.

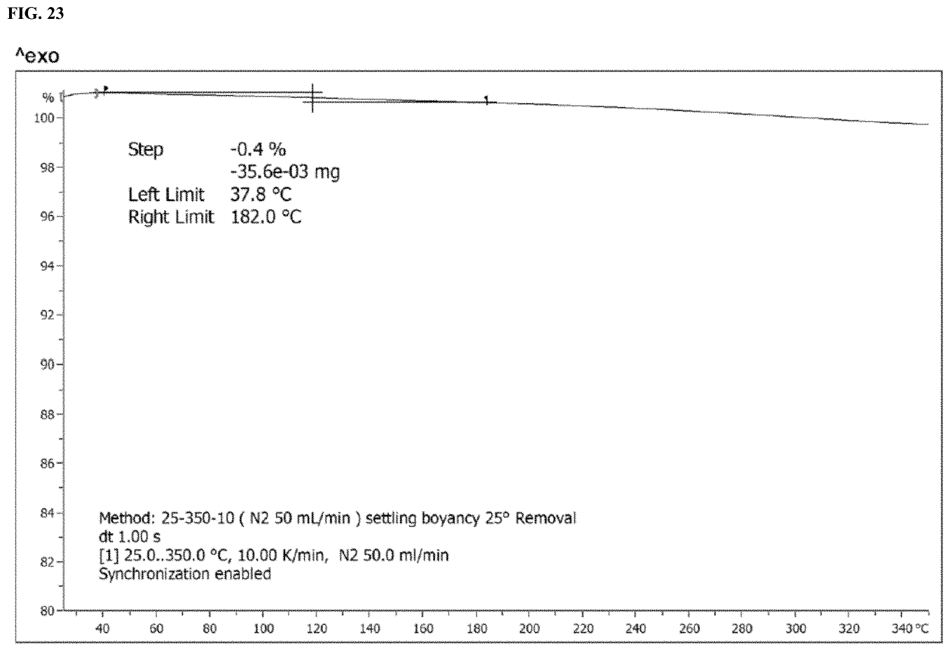

FIG. 23 shows thermogravimetric analysis (TGA) thermogram of Crystalline Form 6.

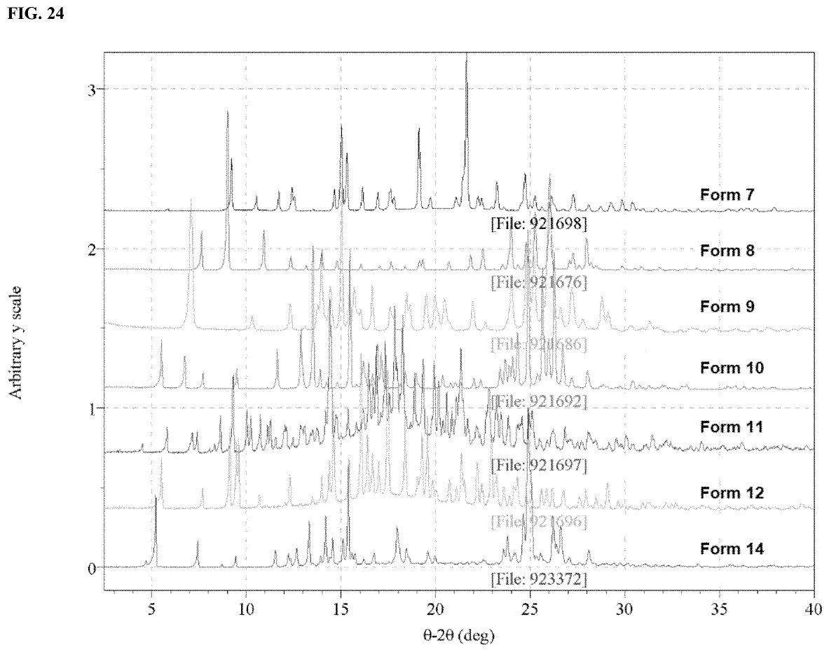

FIG. 24 shows overlay of X-ray powder diffraction (XRPD) patterns of crystalline Compound I Form 7, Form 8, Form 9, Form 10, Form 11, Form 12, and Form 14.

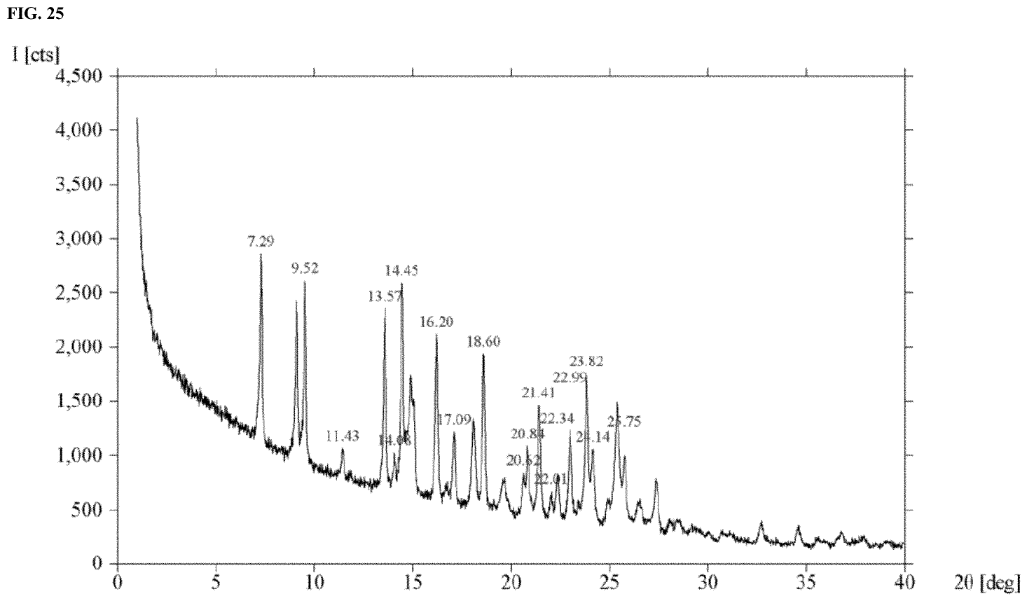

FIG. 25 shows an X-ray powder diffraction (XRPD) pattern of crystalline Compound I Form 13.

### DETAILED DESCRIPTION OF THE DISCLOSURE

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Following long-standing patent law convention, the terms “a”, “an”, and “the” refer to “one or more” when used in this application, including the claims. Thus, for example, reference to “a carrier” includes mixtures of one or more carriers, two or more carriers, and the like.

The term “compound(s) of the present invention” or “present compound(s)” refers to 2-(5-fluoro-2-methyl-1H-indol-3-yl)-1H-imidazo[4,5-f][1,10]phenanthroline (Compound I), or a salt, or a solvate thereof.

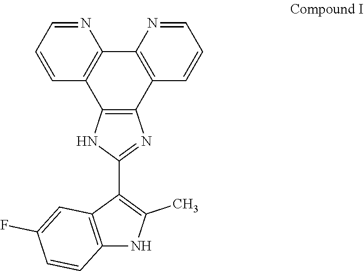 [SMILES: Cc1[nH]c2ccc(F)cc2c1-c1nc2c3cccnc3c3ncccc3c2[nH]1]

Polymorphism can be characterized as the ability of a compound to crystallize into different crystal forms, while maintaining the same chemical formula. Different polymorphs of the same compound (same chemical formula) exists in different crystalline phases that have different arrangements and/or conformation of the molecule in the crystal lattice. As used herein, a polymorph includes crystalline form of a compound (including Compound I) as well as its salts, solvates or hydrates. Polymorphism can affect one or more physical properties, such as stability, solubility, melting point, bulk density, flow properties, bioavailability, etc.

The term “impurity” of a compound, as used herein, means chemicals other than the compound, including, derivatives of the compound, or degradants of the compound that remain with the compound due to incomplete purification, or that develop over time, such as during stability testing, formulation development of the compound or storage of the compound.

The term “chemical purity” of a compound, as used herein, refers to the purity of a compound from other distinct chemical entities. For example, crystalline Compound I having 90% chemical purity means that the crystalline form contains less than 10% of molecules or chemical entity different from Compound I, including synthetic byproducts, residual solvents, or residual organic or inorganic substances.

The term “polymorphic purity” of a compound, as used herein, refers to the purity of a compound to exist in one distinct polymorphic form. For example, Compound I Form 2 having a polymorphic purity of 90% means that the crystalline form contains less than 10% of other polymorphic forms of Compound I in total, such as Form 1.

The term “isomer” refers to compounds having the same chemical formula but may have different stereochemical formula, structural formula, or special arrangements of atoms. Examples of isomers include stereoisomers, diastereomers, enantiomers, conformational isomers, rotamers, geometric isomers, and atropisomers.

The term “composition” denotes one or more substance in a physical form, such as solid, liquid, gas, or a mixture thereof. One example of composition is a pharmaceutical composition, i.e., a composition related to, prepared for, or used in medical treatment. The term “formulation” is also used to indicate one or more substance in a physical form, such as solid, liquid, gas, or a mixture thereof.

The term “co-administration” or “coadministration” refers to administration of a formulation or a composition comprising (a) a compound of the invention or a formulation prepared from a compound of the invention; and (b) one or more additional therapeutic agent and/or radio therapy, in combination, i.e., together in a coordinated fashion.

As used herein, “pharmaceutically acceptable” means suitable for use in contact with the tissues of humans and animals without undue toxicity, irritation, allergic response, and the like, commensurate with a reasonable benefit/risk ratio, and effective for their intended use within the scope of sound medical judgment.

“Salts” include derivatives of an active agent, wherein the active agent is modified by making acid or base addition salts thereof. Preferably, the salts are pharmaceutically acceptable salts. Such salts include, but are not limited to, pharmaceutically acceptable acid addition salts, pharmaceutically acceptable base addition salts, pharmaceutically acceptable metal salts, ammonium and alkylated ammonium salts. Acid addition salts include salts of inorganic acids as well as organic acids. Representative examples of suitable inorganic acids include hydrochloric, hydrobromic, hydroiodic, phosphoric, sulfuric, nitric acids and the like. Representative examples of suitable organic acids include formic, acetic, trichloroacetic, trifluoroacetic, propionic, benzoic, cinnamic, citric, fumaric, glycolic, lactic, maleic, malic, malonic, mandelic, oxalic, picric, pyruvic, salicylic, succinic, methanesulfonic, ethanesulfonic, tartaric, ascorbic, pamoic, bismethylene salicylic, ethanedisulfonic, gluconic, citraconic, aspartic, stearic, palmitic, EDTA, glycolic, p-aminobenzoic, glutamic, benzenesulfonic, p-toluenesulfonic acids, sulphates, nitrates, phosphates, perchlorates, borates, acetates, benzoates, hydroxynaphthoates, glycerophosphates, ketoglutarates and the like. Base addition salts include but are not limited to, ethylenediamine, N-methyl-glucamine, lysine, arginine, ornithine, choline, N,N′-dibenzylethylenediamine, chloroprocaine, diethanolamine, procaine, N-benzylphenethylamine, diethylamine, piperazine, tris-(hydroxymethyl)-aminomethane, tetramethylammonium hydroxide, triethylamine, dibenzylamine, ephenamine, dehydroabietylamine, N-ethylpiperidine, benzylamine, tetramethylammonium, tetraethylammonium, methylamine, dimethylamine, trimethylamine, ethylamine, basic amino acids, e. g., lysine and arginine dicyclohexylamine and the like. Examples of metal salts include lithium, sodium, potassium, magnesium salts and the like. Examples of ammonium and alkylated ammonium salts include ammonium, methylammonium, dimethylammonium, trimethylammonium, ethylammonium, hydroxyethylammonium, diethylammonium, butylammonium, tetramethylammonium salts and the like. Examples of organic bases include lysine, arginine, guanidine, diethanolamine, choline and the like. Standard methods for the preparation of pharmaceutically acceptable salts and their formulations are well known in the art, and are disclosed in various references, including for example, “Remington: The Science and Practice of Pharmacy”, A. Gennaro, ed., 20th edition, Lippincott, Williams & Wilkins, Philadelphia, Pa.

As used herein, “solvate” means a complex formed by solvation (the combination of solvent molecules with molecules or ions of the compounds of the present invention), or an aggregate that consists of a solute ion or molecule (the compounds of the present invention) with one or more solvent molecules. In the present invention, the preferred solvate is hydrate. Examples of hydrate include, but are not limited to, hemihydrate, monohydrate, dihydrate, trihydrate, tetrahydrate, hexahydrate, etc. It should be understood by one of ordinary skill in the art that the pharmaceutically acceptable salt of the present compound may also exist in a solvate form. The solvate is typically formed via hydration which is either part of the preparation of the present compound or through natural absorption of moisture by the anhydrous compound of the present invention. Solvates including hydrates may be consisting in stoichiometric ratios, for example, with two, three, four salt molecules per solvate or per hydrate molecule. Another possibility, for example, that two salt molecules are stoichiometric related to three, five, seven solvent or hydrate molecules. Solvents used for crystallization, such as alcohols, especially methanol and ethanol; aldehydes; ketones, especially acetone; esters, e.g. ethyl acetate; may be embedded in the crystal grating. Preferred are pharmaceutically acceptable solvents.

The term “substantially similar” as used herein with regards to bioavailability of pharmacokinetics means that the two or more therapeutically active agents or drugs provide the same therapeutic effects in a subject.

The term “substantially similar” as used herein with regards to an analytical spectrum, such as XRPD patterns, Raman spectroscopy, etc., means that a spectrum resembles the reference spectrum to a great degree in both the peak locations and their intensity.

The term “substantially free of” as used herein, means free from therapeutically effective amounts of compounds when administered in suggested doses, but may include trace amounts of compounds in non-therapeutically effective amounts.

The terms “excipient”, “carrier”, and “vehicle” are used interchangeably throughout this application and denote a substance with which a compound of the present invention is administered.

“Therapeutically effective amount” means the amount of a therapeutically active agent, when administered to a patient for treating a disease or other undesirable medical condition, is sufficient to have a beneficial effect with respect to that disease or condition. The therapeutically effective amount will vary depending on the identity of the therapeutically active agent, the disease or condition and its severity, and the age, weight, etc. of the patient to be treated. Determining the therapeutically effective amount of the therapeutically active agent is within the ordinary skill of the art and requires no more than routine experimentation.

As used herein, the terms “additional pharmaceutical agent” or “additional therapeutic agent” or “additional therapeutically active agent” with respect to the compounds described herein refers to an active agent other than the Compound I, or a pharmaceutically acceptable salt, ester, or solvate thereof, which is administered to elicit a therapeutic effect. The pharmaceutical agent(s) may be directed to a therapeutic effect related to the condition that the compounds of the present disclosure is intended to treat or ameliorate (e.g., cancer) or, the pharmaceutical agent may be intended to treat or ameliorate a symptom of the underlying condition (e.g., tumor growth, hemorrhage, ulceration, pain, enlarged lymph nodes, cough, jaundice, swelling, weight loss, cachexia, sweating, anemia, paraneoplastic phenomena, thrombosis, etc.) or to further reduce the appearance or severity of side effects of the compounds of the present disclosure.

As used herein, the phrase “a disorder characterized by cell proliferation” or “a condition characterized by cell proliferation” include, but are not limited to, cancer, benign and malignant tumors. Examples of cancer and tumors include, but are not limited to, cancers or tumor growth of the colorectum, breast (including inflammatory breast cancer), lung, liver, pancreas, lymph node, colon, prostate, brain, head and neck, skin, kidney, osteosarcoma, blood and heart (e.g., leukemia, lymphoma, and carcinoma).

The term “treating” means one or more of relieving, alleviating, delaying, reducing, improving, or managing at least one symptom of a condition in a subject. The term “treating” may also mean one or more of arresting, delaying the onset (i.e., the period prior to clinical manifestation of the condition) or reducing the risk of developing or worsening a condition.

The term “patient” or “subject” as used herein, includes humans and animals, preferably mammals.

As used herein, the terms “inhibiting” or “reducing” cell proliferation is meant to slow down, to decrease, or, for example, to stop the amount of cell proliferation, as measured using methods known to those of ordinary skill in the art, by, for example, 10%, 20%, 30%, 40%, 50%, 60%, 70%, 80%, 90%, 95%, or 100%, when compared to proliferating cells that are not subjected to the methods and compositions of the present application.

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application.

### Compound I

Compound I is a small molecule that inhibits expression of the c-Myc oncogene, leading to cell cycle arrest and programmed cell death (apoptosis) in human-derived solid tumor and hematologic cancer cells. Likewise, in nonclinical pharmacology studies Compound I demonstrated in vivo anti-tumor activity against xenograft models of solid tumors and hematologic cancers, with acute myeloid leukemia (AML) cells exhibiting a particular sensitivity to Compound I.

Crystallization and salt formation studies led to discoveries of different crystalline materials for Compound I or a salt or a solvate thereof having different physical properties.

In one embodiment, the present disclosure relates to Compound I free base tetrahydrate. In one embodiment, Compound I free base tetrahydrate is crystalline. In one embodiment, Compound I free base tetrahydrate is not crystalline.

Crystalline Materials of Compound I or a Salt and/or a Solvate Thereof

In one embodiment, the present disclosure provides a crystalline form of Compound I or a salt or a solvate thereof. In one embodiment, the present disclosure provides a crystalline form of Compound I or a pharmaceutically acceptable salt or a solvate thereof.

In one embodiment, the present disclosure provides a crystalline form of a salt and/or solvate of Compound I. In one embodiment, the present disclosure provides a crystalline form of a solvate of Compound I. In one embodiment, the solvate is a hydrate. In one embodiment, Compound I is a monohydrate. In another embodiment, Compound I is a dihydrate. In some embodiments, Compound I is a trihydrate. In other embodiments, Compound I is a tetrahydrate. In one embodiment, Compound I is a pentahydrate. In another embodiment, the solvate is hydrate where the ratio of Compound I and water (H₂O) is from about 1:1 to about 1:5. In one embodiment, Compound I is a free base.

In one embodiment, the crystalline form of the present disclosure relates to Compound I free base tetrahydrate.

In one embodiment, the present disclosure provides a crystalline form of a salt of Compound I. In one embodiment, the salt is a hydrochloric acid (HCl) addition salt. In one embodiment, Compound I is a mono-HCl salt. In another embodiment, Compound I is a bis-HCl salt.

In one embodiment, the crystalline forms are characterized by the interlattice plane intervals determined by an X-ray powder diffraction (XRPD) pattern. The spectrum of XRPD is typically represented by a diagram plotting the intensity of the peaks versus the location of the peaks, i.e., diffraction angle 20 (two-theta) in degrees. The intensities are often given in parenthesis with the following abbreviations: very strong=vst; strong=st; medium=m; weak=w; and very weak=vw. The characteristic peaks of a given XRPD can be selected according to the peak locations and their relative intensity to conveniently distinguish this crystalline structure from others. The % intensity of the peaks relative to the most intense peak may be represented as I/Io.

Those skilled in the art recognize that the measurements of the XRPD peak locations and/or intensity for a given crystalline form of the same compound will vary within a margin of error. The values of degree 20 allow appropriate error margins. Typically, the error margins are represented by “±”. For example, the degree 20 of about “8.7±0.3” denotes a range from about 8.7+0.3, i.e., about 9.0, to about 8.7±0.3, i.e., about 8.4. Depending on the sample preparation techniques, the calibration techniques applied to the instruments, human operational variation, and etc., those skilled in the art recognize that the appropriate error of margins for a XRPD can be about ±1.0; ±0.9; ±0.8; ±0.7; ±0.6; ±0.5; ±0.4; ±0.3; ±0.2; ±0.1; ±0.05; or less.

Additional details of the methods and equipment used for the XRPD analysis are described in the Examples section.

In one embodiment, the crystalline forms are characterized by Differential Scanning calorimetry (DSC). The DSC thermogram is typically expressed by a diagram plotting the normalized heat flow in units of Watts/gram (“W/g”) versus the measured sample temperature in degree C. The DSC thermogram is usually evaluated for extrapolated onset and end (outset) temperatures, peak temperature, and heat of fusion. A peak characteristic value of a DSC thermogram is often used as the characteristic peak to distinguish this crystalline structure from others.

Those skilled in the art recognize that the measurements of the DSC thermogram for a given crystalline form of the same compound will vary within a margin of error. The values of a single peak characteristic value, expressed in degree C., allow appropriate error margins. Typically, the error margins are represented by “±”. For example, the single peak characteristic value of about “53.09±2.0” denotes a range from about 53.09+2.0, i.e., about 55.09, to about 53.09-2.0, i.e., about 51.09. Depending on the sample preparation techniques, the calibration techniques applied to the instruments, human operational variations, and etc., those skilled in the art recognize that the appropriate error of margins for a single peak characteristic value can be ±2.5; ±2.0; ±1.5; ±1.0; ±0.5; or less.

Additional details of the methods and equipment used for the DSC thermogram analysis are described in the Examples section.

In one embodiment, the crystalline forms are characterized by Raman spectroscopy. The Raman spectrum is typically represented by a diagram plotting the Raman intensity of the peaks versus the Raman shift of the peaks. The “peaks” of Raman spectroscopy are also known as “absorption bands”. The intensities are often given in parenthesis with the following abbreviations: strong=st; medium=m; and weak=w. The characteristic peaks of a given Raman spectrum can be selected according to the peak locations and their relative intensity to conveniently distinguish this crystalline structure from others.

In some embodiments, the compound of the present invention has a chemical purity greater than about 50%, about 60%, about 70%, about 80%, about 85%, about 95%, about 98%, or any values in between (i.e., greater than about 83%, greater than about 97%, etc.). In some embodiments, the compound of the present invention has a chemical purity greater than about 80%, about 81%, about 82%, about 83%, about 84%, about 85%, about 86%, about 87%, about 88%, about 89%, about 90%, about 91%, about 92%, about 93%, about 94%, or about 95%. In some embodiments, the compound of the present invention has a chemical purity greater than about 90%. In some embodiments, the compound of the present invention has a chemical purity greater than about 95%. In some embodiments, the compound of the present invention has a chemical purity greater than about 98%. In some embodiments, the compound of the present invention has a chemical purity greater than about 99%.

In some embodiments, the compound of the present invention has a polymorphic purity greater than about 50%, about 55%, about 60%, about 65%, about 70%, about 75% about 80%, about 85%, about 90%, about 95%, about 98%, or any values in between. In some embodiments, the compound of the present invention has a polymorphic purity greater than about 80%, about 81%, about 82%, about 83%, about 84%, about 85%, about 86%, about 87%, about 88%, about 89%, about 90%, about 91%, about 92%, about 93%, about 94%, or about 95%. In some embodiments, the compound of the present invention has a polymorphic purity greater than about 90%. In some embodiments, the compound of the present invention has a polymorphic purity greater than about 95%. In some embodiments, the compound of the present invention has a polymorphic purity greater than about 98%. In some embodiments, the compound of the present invention has a polymorphic purity greater than about 99%.

Additional characterization and methods of characterization the compound of the present invention are described below and in the Examples.

### Compound I Solvates, Hydrates, and Anhydrates

In one embodiment, the crystalline form of Compound I is a free base acetate solvate (Compound I-acetate). In some embodiments, Compound I-acetate is also a hydrate. In one embodiment, the crystalline form of Compound I is a free base hydrate (Compound I-hydrate). In one embodiment, crystalline form of Compound I-acetate and Compound I-hydrate exhibits different polymorphs, which are but not limited to, Compound I-acetate Form 1 and Compound I-hydrate Form 2 (Compound I free base tetrahydrate), as defined in the following sections.

In one embodiment, the crystalline form of Compound I is Form 1, Form 2, Form 3, Form 4, Form 5, Form 6, Form 7, Form 8, Form 9, Form 10, Form 11, Form 12, Form 13, or Form 14. In one embodiment, the crystalline form of Compound I is Form 2.

In one embodiment of the present disclosure, the crystalline form of Compound I may comprise of a mixture of one or more forms of polymorphs of Compound I or a salt and/or solvate thereof and/or Compound I-hydrate and/or Compound I-acetate. In some embodiments, the crystalline form of Compound I-acetate may comprise of substantially pure form of one polymorph type. In one embodiment, the crystalline form of Compound I-acetate may comprise of over about 99.9%, about 99.8%, about 99.7%, about 99.6%, about 99.5%, about 99.4%, about 99.3%, about 99.2%, about 99.1%, or about 99.0% of one polymorph of Compound I-acetate. In another embodiment, the crystalline form of Compound I-acetate may comprise over about 99%, 98%, 97%, 96%, 95%, 94%, 93%, 92%, 91%, or 90% of one polymorph of Compound I-acetate. In some embodiments, the crystalline form of Compound I-acetate may comprise over about 90%, 85%, 80%, 75%, 70%, 65%, 60%, 55%, 50%, 45%, or 40% of one polymorph of Compound I-acetate.

In one embodiment, the crystalline form of Compound I-hydrate may comprise of over about 99.9%, about 99.8%, about 99.7%, about 99.6%, about 99.5%, about 99.4%, about 99.3%, about 99.2%, about 99.1%, or about 99.0% of one polymorph of Compound I-hydrate. In another embodiment, the crystalline form of Compound I-hydrate may comprise over about 99%, 98%, 97%, 96%, 95%, 94%, 93%, 92%, 91%, or 90% of one polymorph of Compound I-hydrate. In some embodiments, the crystalline form of Compound I-solvate may comprise over about 90%, 85%, 80%, 75%, 70%, 65%, 60%, 55%, 50%, 45%, or 40% of one polymorph of Compound I-hydrate.

In one embodiment of the present disclosure, the crystalline form of Compound I may comprise of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, about 90%, about 85%, about 80%, about 75%, about 70%, about 65%, about 60%, about 55% or about 50% of crystalline Compound I-acetate Form 1.

In one embodiment of the present disclosure, the crystalline form can be crystalline Compound I-acetate Form 1 comprising about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45% or 50% of crystalline Compound I-hydrate Form 2 (Compound I free base tetrahydrate).

In one embodiment of the present disclosure, the crystalline form of Compound I can be crystalline Compound I-acetate Form 1 comprising about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45% or 50% of crystalline Compound I-hydrate Form 2.

In one embodiment of the present disclosure, the crystalline form of Compound I can comprise of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, about 90%, about 85%, about 80%, about 75%, about 70%, about 65%, about 60%, about 55% or about 50% of crystalline Compound I-hydrate Form 2.

In one embodiment of the present disclosure, the crystalline form of Compound I can be crystalline Compound I-hydrate Form 2 comprising about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45% or 50% of crystalline Compound I-acetate Forms 1.

In one embodiment of the present disclosure, the crystalline form of Compound I can be crystalline Compound I-hydrate Form 2 comprising about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45% or 50% of crystalline Compound I-acetate Form 1.

In one embodiment, the present disclosure relates to a Compound I hydrate or solvate. In one embodiment, the Compound I hydrate or solvate is crystalline. In one embodiment, the Compound I hydrate or solvate is not crystalline.

### Crystalline Compound I-Acetate Form 1

In one embodiment, crystalline Compound I-acetate Form 1 (Crystalline Form 1) comprises about 4% to about 10% H₂O content by weight (wt %). In one embodiment, Form 1 comprises about 4%, about 5%, about 6%, about 7%, about 8%, about 9%, or about 10% H₂O content by weight. In another embodiment, Form 1 comprises about 4.5%, about 5%, or about 5.5% H₂O content by weight. In one embodiment, Crystalline Form 1 is a monohydrate.

In one embodiment, Crystalline Form 1 of Compound I-acetate exhibits an XRPD comprising one or more peaks at about 10.0, 11.5, and 13 degrees two-theta with the margin of error of about ±0.5; about ±0.4; about ±0.3; about ±0.2; about ±0.1; about ±0.05; or less. In another embodiment, the XRPD of the Crystalline Form 1 further comprises one or more peaks at about 27 degrees two-theta with the margin of error of about ±0.5; about ±0.4; about ±0.3; about ±0.2; about ±0.1; about ±0.05; or less.

In one specific embodiment, the Crystalline Form 1 exhibits an XRPD that is substantially similar to FIG. 1 (top line).

In one embodiment, the Crystalline Form 1 exhibits a DSC thermogram comprising a sharp endotherm at about 206.5° C. with the error of margin of about ±2.5; about ±2.0; about ±1.5; about ±1.0; about ±0.5; or less. In one embodiment, the Crystalline Form 1 exhibits a DSC thermogram comprising a broad exotherm at about 175.9° C. with the error of margin of about ±2.5; about ±2.0; about ±1.5; about ±1.0; about ±0.5; or less. In one specific embodiment, the Crystalline Form 1 exhibits a DSC thermogram that is substantially similar to FIG. 2 (top line of the bottom set).

In one embodiment, the Crystalline Form 1 exhibits a TGA thermogram that is substantially similar to FIG. 2 (top line of the top set). In other embodiments, the TGA thermogram of the Crystalline Form 1 exhibits a weight loss of about 0.0 to about 10% in the temperature range of 25° C. to 250° C. In other embodiments, the TGA thermogram of the Crystalline Form 1 exhibits a weight loss of about 7% to about 10% in the temperature range of 25° C. to 195° C. In other embodiments, the TGA thermogram of the Crystalline Form 1 exhibits a weight loss of about 0.2% to about 2.0% in the temperature range of 195° C. to 230° C.

In one embodiment, the present disclosure relates to Compound I acetate. In one embodiment, the present disclosure relates to Compound I acetate hydrate.

### Crystalline Compound I-Hydrate Form 2 (Compound I Free Base Tetrahydrate)

In one embodiment, a crystalline Compound I-hydrate Form 2 (Crystalline Form 2) comprises about 14% to about 18% H₂O content by weight (wt %). In one embodiment, Form 2 comprises about 14%, about 15%, about 16%, about 17%, or about 18% H₂O content by weight. In another embodiment, Form 2 comprises about 15%, about 15.5%, about 16%, about 16.5%, or about 17% H₂O content by weight. In some embodiments, water content is measured by Karl Fischer analysis.

In one embodiment, the Crystalline Form 2 of Compound I-hydrate exhibits an XRPD comprising one or more peaks at about 10.0 and about 25.0 degrees two-theta with the margin of error of about ±0.5; about ±0.4; about ±0.3; about ±0.2; about ±0.1; about ±0.05; or less. In another embodiment, the XRPD of the Crystalline Form 2 further comprises one or more peaks at about 26.3 and about 28.2 degrees two-theta with the margin of error of about ±0.5; about ±0.4; about ±0.3; about ±0.2; about ±0.1; about ±0.05; or less. In further embodiment, the Crystalline Form 2 further comprises one or more peaks at about 6.0, about 9.4, and about 25.2 degrees two-theta with the margin of error of about ±0.5; about ±0.4; about ±0.3; about ±0.2; about ±0.1; about ±0.05; or less. In one specific embodiment, the Crystalline Form 2 exhibits an XRPD that is substantially similar to FIG. 3.

In one embodiment, the Crystalline Form 2 exhibits an XRPD pattern comprising peaks at 10.0±0.2 and 25.0±0.2 degrees two-theta. In one embodiment, the Crystalline Form 2 exhibits an XRPD pattern comprising peaks at 10.0±0.2, 25.0±0.2, and 26.3±0.2 degrees two-theta. In one embodiment, the Crystalline Form 2 exhibits an XRPD pattern comprising peaks at 10.0±0.2, 25.0±0.2, 26.3±0.2, and 28.2±0.2 degrees two-theta. In one embodiment, the Crystalline Form 2 exhibits an XRPD pattern comprising peaks at 10.0±0.2, 25.0±0.2, 25.2±0.2, 26.3±0.2, and 28.2±0.2 degrees two-theta. In one embodiment, the Crystalline Form 2 exhibits an XRPD pattern comprising peaks at 6.0±0.2, 10.0±0.2, 25.0±0.2, 25.2±0.2, 26.3±0.2, and 28.2±0.2 degrees two-theta. In one embodiment, the Crystalline Form 2 exhibits an XRPD pattern comprising peaks at 6.0±0.2, 9.4±0.2, 10.0±0.2, 25.0±0.2, 25.2±0.2, 26.3±0.2, and 28.2±0.2 degrees two-theta. In one embodiment, the Crystalline Form 2 exhibits an XRPD pattern comprising peaks at 6.0±0.2, 9.4±0.2, 10.0±0.2, 12.0±0.2, 25.0±0.2, 25.2±0.2, 26.3±0.2, and 28.2±0.2 degrees two-theta.

In one embodiment, the Crystalline Form 2 of Compound I-hydrate exhibits an XRPD spectrum comprising peaks shown in Table A1. In one embodiment, the Crystalline Form 2 exhibits an XRPD spectrum comprising peaks shown in Table A2. In one embodiment, the Crystalline Form 2 exhibits an XRPD spectrum comprising all peaks in Table A1 having at least 30% intensity, with the understanding that some of the close peaks can form one broad peak. In one embodiment, the Crystalline Form 2 exhibits an XRPD spectrum comprising all peaks in Table A2 having at least 40% intensity, with the understanding that some of the close peaks can form one broad peak. In one embodiment, the Crystalline Form 2 exhibits an XRPD spectrum comprising all peaks in Table A2 having at least 30% intensity, with the understanding that some of the close peaks can form one broad peak. In one embodiment, the Crystalline Form 2 exhibits an XRPD spectrum comprising all peaks in Table A2 having at least 25% intensity, with the understanding that some of the close peaks can form one broad peak.

TABLE A1
XRPD data for the Crystalline Form 2
of Compound I-hydrate

| °2θ | d space (Å) | Intensity (%) |
|---|---|---|
| 6.00 ± 0.20 | 14.718 ± 0.490 | 28 |
| 9.39 ± 0.20 | 9.411 ± 0.200 | 26 |
| 10.03 ± 0.20 | 8.812 ± 0.175 | 100 |
| 12.03 ± 0.20 | 7.351 ± 0.122 | 24 |
| 12.19 ± 0.20 | 7.255 ± 0.119 | 9 |
| 12.53 ± 0.20 | 7.059 ± 0.112 | 8 |
| 13.07 ± 0.20 | 6.768 ± 0.103 | 5 |
| 13.62 ± 0.20 | 6.496 ± 0.095 | 6 |
| 13.95 ± 0.20 | 6.343 ± 0.090 | 9 |
| 14.79 ± 0.20 | 5.985 ± 0.080 | 6 |
| 15.31 ± 0.20 | 5.783 ± 0.075 | 8 |
| 16.80 ± 0.20 | 5.273 ± 0.062 | 9 |
| 18.56 ± 0.20 | 4.777 ± 0.051 | 16 |
| 18.86 ± 0.20 | 4.701 ± 0.049 | 13 |
| 19.41 ± 0.20 | 4.569 ± 0.047 | 11 |
| 20.16 ± 0.20 | 4.401 ± 0.043 | 11 |
| 20.55 ± 0.20 | 4.318 ± 0.042 | 13 |
| 21.01 ± 0.20 | 4.225 ± 0.040 | 13 |
| 21.55 ± 0.20 | 4.120 ± 0.038 | 14 |
| 22.18 ± 0.20 | 4.005 ± 0.036 | 15 |
| 22.98 ± 0.20 | 3.867 ± 0.033 | 13 |
| 23.19 ± 0.20 | 3.832 ± 0.033 | 18 |
| 24.06 ± 0.20 | 3.696 ± 0.030 | 12 |
| 24.57 ± 0.20 | 3.620 ± 0.029 | 12 |
| 25.01 ± 0.20 | 3.558 ± 0.028 | 71 |
| 25.24 ± 0.20 | 3.526 ± 0.027 | 29 |
| 25.78 ± 0.20 | 3.453 ± 0.026 | 17 |
| 26.28 ± 0.20 | 3.388 ± 0.025 | 47 |
| 26.93 ± 0.20 | 3.308 ± 0.024 | 14 |
| 28.18 ± 0.20 | 3.164 ± 0.022 | 32 |
| 28.89 ± 0.20 | 3.088 ± 0.021 | 15 |
| 29.41 ± 0.20 | 3.035 ± 0.020 | 8 |
| 29.85 ± 0.20 | 2.991 ± 0.020 | 9 |
| 30.39 ± 0.20 | 2.939 ± 0.019 | 11 |
| 30.79 ± 0.20 | 2.902 ± 0.018 | 10 |
| 31.10 ± 0.20 | 2.873 ± 0.018 | 8 |
| 31.82 ± 0.20 | 2.810 ± 0.017 | 7 |
| 32.27 ± 0.20 | 2.772 ± 0.017 | 9 |

TABLE A2
XRPD data for the Crystalline
Form 2 of Compound I-hydrate

| °2θ | d space (Å) | Intensity (%) |
|---|---|---|
| 6.00 ± 0.20 | 14.718 ± 0.490 | 28 |
| 9.39 ± 0.20 | 9.411 ± 0.200 | 26 |
| 10.03 ± 0.20 | 8.812 ± 0.175 | 100 |
| 12.03 ± 0.20 | 7.351 ± 0.122 | 24 |
| 25.01 ± 0.20 | 3.558 ± 0.028 | 71 |
| 25.24 ± 0.20 | 3.526 ± 0.027 | 29 |
| 26.28 ± 0.20 | 3.388 ± 0.025 | 47 |
| 28.18 ± 0.20 | 3.164 ± 0.022 | 32 |

In one embodiment, the Crystalline Form 2 exhibits a DSC thermogram comprising an exotherm peak (maximum) at about 200° C. to about 220° C. with the error of margin of about ±2.5; about ±2.0; about ±1.5; about ±1.0; about ±0.5; or less. In one embodiment, the Crystalline Form 2 exhibits a DSC thermogram comprising an exotherm peak at about 205° C. to about 207° C. with the error of margin of about ±2.5; about ±2.0; about ±1.5; about ±1.0; about ±0.5; or less. In one embodiment, the Crystalline Form 2 exhibits a DSC thermogram comprising an exotherm peak in between 200° C.±0.5° C. to about 220° C.±0.5° C. In one embodiment, the Crystalline Form 2 exhibits a DSC thermogram comprising an exotherm peak in between 205° C.±0.5° C. to about 207° C.±0.5° C.

In one embodiment, the Crystalline Form 2 exhibits a DSC thermogram comprising at least one broad endotherm between about 60° C. to about 180° C. In one embodiment, the Crystalline Form 2 exhibits a DSC thermogram comprising at least two broad endotherm peaks between about 60° C. to about 180° C. In one embodiment, the Crystalline Form 2 exhibits a DSC thermogram comprising three broad endotherm peaks between about 60° C. to about 180° C.

In one embodiment the Crystalline Form 2 exhibits a DSC thermogram comprising an endotherm peak (minimum) in between 105° C. and about 130° C. with the error of margin of about ±2.5; about ±2.0; about ±1.5; about ±1.0; about ±0.5; or less. In one embodiment the Crystalline Form 2 exhibits a DSC thermogram comprising an endotherm peak in between 115° C. and about 120° C. with the error of margin of about ±2.5; about ±2.0; about ±1.5; about ±1.0; about ±0.5; or less. In one embodiment the Crystalline Form 2 exhibits a DSC thermogram comprising an endotherm peak in between 105° C.±1° C. to about 130° C.±1° C. In one embodiment the Crystalline Form 2 exhibits a DSC thermogram comprising an endotherm peak in between 115° C.±1° C. to about 118° C.±1° C.

In one embodiment the Crystalline Form 2 exhibits a DSC thermogram comprising an endotherm peak in between 140° C. and about 170° C. with the error of margin of about ±2.5; about ±2.0; about ±1.5; about ±1.0; about ±0.5; or less. In one embodiment the Crystalline Form 2 exhibits a DSC thermogram comprising an endotherm peak in between 148° C. and about 156° C. with the error of margin of about ±2.5; about ±2.0; about ±1.5; about ±1.0; about ±0.5; or less. In one embodiment the Crystalline Form 2 exhibits a DSC thermogram comprising an endotherm peak in between 140° C.±1° C. to about 170° C.±1° C. In one embodiment the Crystalline Form 2 exhibits a DSC thermogram comprising an endotherm peak in between 150° C.±1° C. to about 155° C.±1° C.

In one specific embodiment, the Crystalline Form 2 exhibits a DSC thermogram that is substantially similar to FIG. 4. In another embodiment, the Crystalline Form 2 exhibits a DSC thermogram that is substantially similar to FIG. 5. In some embodiments, the Crystalline Form 2 exhibits a DSC thermogram that is substantially similar to FIG. 13.

In one embodiment, the Crystalline Form 2 exhibits a TGA thermogram that is substantially similar to FIG. 6. In one embodiment, the Crystalline Form 2 exhibits a TGA thermogram that is substantially similar to FIG. 12. In other embodiments, the TGA thermogram of the Crystalline Form 2 exhibits a weight loss of about 0.0 to about 20% in the temperature range of 25° C. to 250° C. In other embodiments, the TGA thermogram of the Crystalline Form 2 exhibits a weight loss of about 14% to about 18% in the temperature range of 25° C. to 250° C. In other embodiments, the TGA thermogram of the Crystalline Form 2 exhibits a weight loss of about 8% to about 12% in the temperature range of 30° C. to 137° C. In other embodiments, the TGA thermogram of the Crystalline Form 2 exhibits a weight loss of about 8% to about 12% in the temperature range of 30° C. to 130° C. In other embodiments, the TGA thermogram of the Crystalline Form 2 exhibits a weight loss of about 2.0% to about 8.0% in the temperature range of 137° C. to 190° C. In other embodiments, the TGA thermogram of the Crystalline Form 2 exhibits a weight loss of about 2.0% to about 8.0% in the temperature range of 130° C. to 190° C.

In one embodiment, Crystalline Form 2 exhibits a dynamic vapor sorption (DVS) sorption and de-sorption substantially similar to FIG. 7. In another embodiment, Crystalline Form 2 exhibits a DVS isotherm substantially similar to FIG. 8. FIGS. 7 and 8 shows continuous isothermal adsorption of water with a total mass uptake of 12%. The mass uptake of 12% is equal to three equivalents of water; however, the material was not fully dry yet after drying at 0% RH for 24 hours. Karl Fischer Coulometry data show 16 mass % water, which corresponds to the theoretical value of a tetrahydrate; 16%. The water uptake and release is not stepwise, and no indication of other hydrates with different ratios of water is present.

The de-sorption cycle gives a stable signal down to 10% RH, which demonstrates that Crystalline Form 2 is very stable. An anhydrate Compound I from Crystalline Form 2 could not be obtained at 0% RH or at full vacuum for 24 hours. Under these drying conditions the monohydrate is the stable form. In one embodiment, Crystalline Form 1 is the stable form.

In some embodiments, Crystalline Form 2 exhibits a DVS isotherm substantially similar to FIG. 14.

In one embodiment, Crystalline Form 1 transforms to Crystalline Form 2 in the presence of water and/or humidity. In one embodiment, at 20° C., only 1% water is needed for the transition of Crystalline Form 1 into Crystalline Form 2. In another embodiment, at 50° C., at least 25% water is needed for the transition of Crystalline Form 1 into Crystalline Form 2 (Example 1).

In one embodiment, Crystalline Form 2 transforms to Crystalline Form 3 at below about 11% RH. In one embodiment, Crystalline Form 2 transforms to Crystalline Form 3 at an elevated temperature and/or under vacuum.

In one embodiment, Crystalline Form 2 transforms to Crystalline Form 4 at an elevated temperature. In one embodiment, Crystalline Form 2 transforms to Crystalline Form 4 at about 180° C. to about 220° C.

In some embodiments, Crystalline Form 2 comprises about 15.5% w/w to about 17.0% w/w water. In some embodiments, Crystalline Form 2 comprises about 15.8% w/w to about 16.8% w/w water. In some embodiments, Crystalline Form 2 comprises 16.0%±0.3 w/w water. In some embodiments, Crystalline Form 2 comprises 16.0%±0.2 w/w water. In some embodiments, water content is measured by Karl Fischer analysis.

In one embodiment, Crystalline Form 2 is a stable polymorph in the presence of water.

In one embodiment, Crystalline Form 2 is stable at and above about 11% RH in the solid state.

In one embodiment, the Crystalline Form 2 is substantially pure. In another embodiment, the Crystalline Form 2 is at least 95%, at least 95.5%, at least 96%, at least 96.5%, at least 97%, at least 97.5%, at least 98%, at least 98.5%, or at least 99% chemically pure (w/w %). In one embodiment, the Crystalline Form 2 is substantially pure. In another embodiment, the Crystalline Form 2 is at least about 95%, at least about 95.5%, at least about 96%, at least about 96.5%, at least about 97%, at least about 97.5%, at least about 98%, at least about 98.5%, at least about 99% or at least about 99.5% chemically pure (w/w %). In one embodiment, the Crystalline Form 2 is greater than or equal to 97.5% pure (w/w %). In one embodiment, the Crystalline Form 2 is greater than or equal to about 97.5% pure (w/w %). In one embodiment, the Crystalline Form 2 is greater than or equal to about 98.0% pure (w/w %). In one embodiment, the Crystalline Form 2 is greater than or equal to about 98.5% pure (w/w %). In one embodiment, the Crystalline Form 2 is greater than or equal to about 99.0% pure (w/w %). In one embodiment, the chemical purity is assayed by high performance liquid chromatography (HPLC).

In another embodiment, the Crystalline Form 2 comprises less than 5%, less than 4.5%, less than 4%, less than 3.5%, less than 3%, less than 2.5%, less than 2%, less than 1.5%, less than 1%, or less than 0.5% total impurities (w/w %). In another embodiment, the Crystalline Form 2 comprises less than about 5%, less than about 4.5%, less than about 4%, less than about 3.5%, less than about 3%, less than about 2.5%, less than about 2%, less than about 1.5%, less than about 1%, or less than about 0.5% total impurities (w/w %). In another embodiment, the Crystalline Form 2 comprises less than or equal to 2.5% total impurities (w/w %). In another embodiment, the Crystalline Form 2 comprises less than or equal to 1.0% total impurities (w/w %). In another embodiment, the Crystalline Form 2 comprises less than or equal to 0.5% total impurities (w/w %). In one embodiment, the impurities are measured by HPLC.

In one embodiment, the Crystalline Form 2 comprises less than about 5%, less than about 4.5%, less than about 4%, less than about 3.5%, less than about 3%, less than about 2.5%, less than about 2%, less than about 1.5%, less than about 1%, or less than about 0.5% Crystalline Form 3 (w/w %).

In one embodiment, the Crystalline Form 2 comprises less than 10,000 ppm, less than 7,500 ppm, less than 6,000 ppm, less than 5,000 ppm, less than 4,000 ppm, or less than 3,000 ppm acetone. In one embodiment, the Crystalline Form 2 comprises less than or equal to 5,000 ppm acetone. In some embodiments, the amount of acetone is determined by headspace gas chromatography (HS-GC).

In one embodiment, the Crystalline Form 2 comprises less than 10,000 ppm, less than 7,500 ppm, less than 6,000 ppm, less than 5,000 ppm, less than 4,000 ppm, or less than 3,000 ppm 2-propanol. In one embodiment, the Crystalline Form 2 comprises less than or equal to 5,000 ppm 2-propanol. In some embodiments, the amount of 2-propanol is determined by headspace gas chromatography (HS-GC).

In one embodiment, the Crystalline Form 2 comprises less than 1,500 ppm, less than 1,250 ppm, less than 1,000 ppm, less than 900 ppm, less than 800 ppm, less than 700 ppm or less than 600 ppm tetrahydrofuran. In one embodiment, the Crystalline Form 2 comprises less than or equal to 720 ppm tetrahydrofuran. In some embodiments, the amount of tetrahydrofuran is determined by headspace gas chromatography (HS-GC).

In one embodiment, the Crystalline Form 2 comprises less than 10,000 ppm, less than 7,500 ppm, less than 6,000 ppm, less than 5,000 ppm, less than 4,000 ppm, or less than 3,000 ppm acetic acid. In one embodiment, the Crystalline Form 2 comprises less than or equal to 5,000 ppm acetic acid. In some embodiments, the amount of acetic acid is determined by ion chromatography.

### Crystalline Compound I-Hydrate Form 3 (Compound I Free Base Dihydrate)

In one embodiment, crystalline Compound I-hydrate Form 3 (Crystalline Form 3) comprises about 6% to about 10% H₂O content by weight (wt %). In one embodiment, Form 3 comprises about 6%, about 7%, about 8%, about 9%, or about 10% H₂O content by weight. In another embodiment, Form 3 comprises about 7%, about 7.5%, about 8%, about 8.1%, about 8.2%, about 8.3%, about 8.4%, about 8.5%, about 8.6%, about 8.7%, about 8.8%, about 8.9%, or about 9% H₂O content by weight. In some embodiments, water content is measured by Karl Fischer analysis.

In one embodiment, Crystalline Form 3 of Compound I-hydrate exhibits an XRPD comprising one or more peaks at about 9.6, about 12.6, and about 26.2 degrees two-theta with the margin of error of about ±0.5; about ±0.4; about ±0.3; about ±0.2; about ±0.1; about ±0.05; or less. In another embodiment, the XRPD of the Crystalline Form 3 further comprises one or more peaks at about 24.8 and about 25.5 degrees two-theta with the margin of error of about ±0.5; about ±0.4; about ±0.3; about ±0.2; about ±0.1; about ±0.05; or less. In further embodiment, the Crystalline Form 3 further comprises one or more peaks at about 6.3 and about 28.9 degrees two-theta with the margin of error of about ±0.5; about ±0.4; about ±0.3; about ±0.2; about ±0.1; about ±0.05; or less. In one specific embodiment, the Crystalline Form 3 exhibits an XRPD that is substantially similar to FIG. 15B.

In one embodiment, the Crystalline Form 3 exhibits an XRPD pattern comprising peaks at 9.6±0.2, 12.6±0.2 and 26.2±0.2 degrees two-theta. In one embodiment, the Crystalline Form 3 exhibits an XRPD pattern comprising peaks at 9.6±0.2, 12.6±0.2, 24.8±0.2, 25.5±0.2, and 26.2±0.2 degrees two-theta. In one embodiment, the Crystalline Form 3 exhibits an XRPD pattern comprising peaks at 6.3±0.2, 9.6±0.2, 12.6±0.2, 24.8±0.2, 25.5±0.2, and 26.2±0.2 degrees two-theta. In one embodiment, the Crystalline Form 3 exhibits an XRPD pattern comprising peaks at 6.3±0.2, 9.6±0.2, 12.6±0.2, 24.8±0.2, 25.5±0.2, 26.2±0.2, and 28.9±0.2 degrees two-theta.

In one embodiment, the Crystalline Form 3 of Compound I-hydrate exhibits an XRPD spectrum comprising peaks shown in Table B1. In one embodiment, the Crystalline Form 3 exhibits an XRPD spectrum comprising peaks shown in Table B2. In one embodiment, the Crystalline Form 3 exhibits an XRPD spectrum comprising all peaks in Table B1 having at least 25% intensity, with the understanding that some of the close peaks can form one broad peak. In one embodiment, the Crystalline Form 3 exhibits an XRPD spectrum comprising all peaks in Table B2 having at least 25% intensity, with the understanding that some of the close peaks can form one broad peak. In one embodiment, the Crystalline Form 3 exhibits an XRPD spectrum comprising all peaks in Table B2 having at least 20% intensity, with the understanding that some of the close peaks can form one broad peak. In one embodiment, the Crystalline Form 3 exhibits an XRPD spectrum comprising all peaks in Table B2 having at least 15% intensity, with the understanding that some of the close peaks can form one broad peak.

TABLE B1
XRPD data for the Crystalline Form 3

| °2θ | d space (Å) | Intensity (%) |
|---|---|---|
| 6.27 ± 0.20 | 14.085 ± 0.449 | 18 |
| 9.61 ± 0.20 | 9.196 ± 0.191 | 100 |
| 11.03 ± 0.20 | 8.015 ± 0.145 | 16 |
| 11.94 ± 0.20 | 7.406 ± 0.124 | 5 |
| 12.57 ± 0.20 | 7.036 ± 0.112 | 28 |
| 12.99 ± 0.20 | 6.810 ± 0.104 | 5 |
| 14.29 ± 0.20 | 6.193 ± 0.086 | 7 |
| 15.48 ± 0.20 | 5.720 ± 0.073 | 7 |
| 17.50 ± 0.20 | 5.064 ± 0.057 | 6 |
| 17.85 ± 0.20 | 4.965 ± 0.055 | 7 |
| 18.44 ± 0.20 | 4.808 ± 0.052 | 5 |
| 18.91 ± 0.20 | 4.689 ± 0.049 | 4 |
| 20.52 ± 0.20 | 4.325 ± 0.042 | 8 |
| 20.84 ± 0.20 | 4.259 ± 0.040 | 8 |
| 22.66 ± 0.20 | 3.921 ± 0.034 | 7 |
| 23.43 ± 0.20 | 3.794 ± 0.032 | 4 |
| 23.99 ± 0.20 | 3.707 ± 0.030 | 5 |
| 24.37 ± 0.20 | 3.650 ± 0.029 | 10 |
| 24.83 ± 0.20 | 3.583 ± 0.028 | 22 |
| 25.54 ± 0.20 | 3.485 ± 0.027 | 27 |
| 26.23 ± 0.20 | 3.395 ± 0.025 | 29 |
| 26.80 ± 0.20 | 3.324 ± 0.024 | 13 |
| 27.20 ± 0.20 | 3.276 ± 0.024 | 10 |
| 27.74 ± 0.20 | 3.213 ± 0.023 | 7 |
| 28.87 ± 0.20 | 3.090 ± 0.021 | 17 |

TABLE B2
XRPD data for the Crystalline Form 3

| °2θ | d space (Å) | Intensity (%) |
|---|---|---|
| 6.27 ± 0.20 | 14.085 ± 0.449 | 18 |
| 9.61 ± 0.20 | 9.196 ± 0.191 | 100 |
| 11.03 ± 0.20 | 8.015 ± 0.145 | 16 |
| 12.57 ± 0.20 | 7.036 ± 0.112 | 28 |
| 24.83 ± 0.20 | 3.583 ± 0.028 | 22 |
| 25.54 ± 0.20 | 3.485 ± 0.027 | 27 |
| 26.23 ± 0.20 | 3.395 ± 0.025 | 29 |
| 28.87 ± 0.20 | 3.090 ± 0.021 | 17 |

In one embodiment, the Crystalline Form 3 exhibits a DSC thermogram comprising an exotherm peak (maximum) at about 200° C. to about 220° C. with the error of margin of about ±2.5; about ±2.0; about ±1.5; about ±1.0; about ±0.5; or less. In one embodiment, the Crystalline Form 3 exhibits a DSC thermogram comprising an exotherm peak at about 205° C. to about 210° C. with the error of margin of about ±2.5; about ±2.0; about ±1.5; about ±1.0; about ±0.5; or less. In one embodiment, the Crystalline Form 3 exhibits a DSC thermogram comprising an exotherm peak in between 200° C.±0.5° C. to about 220° C.±0.5° C. In one embodiment, the Crystalline Form 3 exhibits a DSC thermogram comprising an exotherm peak in between 205° C.±0.5° C. to about 210° C.±0.5° C.

In one embodiment, the Crystalline Form 3 exhibits a DSC thermogram comprising at least one broad endotherm between about 60° C. to about 180° C. In one embodiment the Crystalline Form 3 exhibits a DSC thermogram comprising an endotherm peak in between 130° C. and about 160° C. with the error of margin of about ±2.5; about ±2.0; about ±1.5; about ±1.0; about ±0.5; or less. In one embodiment the Crystalline Form 3 exhibits a DSC thermogram comprising an endotherm peak in between 140° C. and about 150° C. with the error of margin of about ±2.5; about ±2.0; about ±1.5; about ±1.0; about ±0.5; or less. In one embodiment the Crystalline Form 3 exhibits a DSC thermogram comprising an endotherm peak in between 130° C.±1° C. to about 160° C.±1° C. In one embodiment the Crystalline Form 3 exhibits a DSC thermogram comprising an endotherm peak in between 140° C.±1° C. to about 150° C.±1° C.

In one specific embodiment, the Crystalline Form 3 exhibits a DSC thermogram that is substantially similar to FIG. 16.

In one embodiment, the Crystalline Form 3 exhibits a TGA thermogram that is substantially similar to FIG. 17. In other embodiments, the TGA thermogram of the Crystalline Form 3 exhibits a weight loss of about 0.0 to about 15% in the temperature range of 25° C. to 250° C. In other embodiments, the TGA thermogram of the Crystalline Form 3 exhibits a weight loss of about 6% to about 10% in the temperature range of 30° C. to 200° C.

In some embodiments, Crystalline Form 3 exhibits a DVS isotherm substantially similar to FIG. 18. In one embodiment, Crystalline Form 3 gains about 11.1 wt % (equivalent to about 2.5 moles of H₂O) between 5 and 95% RH, with the majority of the water uptake (about 9.8 wt %) occurring above 45% RH. In one embodiment, Form 3 upon desorption to 5% RH, the sample retains about 8.8 wt % of the gained moisture, equivalent to two moles of H₂O. In some embodiments, the post-DVS sample contains approximately four moles of H₂O, which is consistent with to the water content of the tetrahydrate Form 2. The XRPD pattern of the post-DVS solids is consistent with Form 2.

In one embodiment, Crystalline Form 2 transforms to Crystalline Form 3 when exposed to low humidity, e.g., over P₂O₅, and dried. In one embodiment, Crystalline Form 3 transforms to Crystalline Form 2 under high humidity or with high water activity. In one embodiment, Crystalline Form 3 transforms to Crystalline Form 2 when exposed to about 59% RH for 11 days. In one embodiment, Crystalline Form 3 transforms to Crystalline Form 2 when exposed to about 75% RH for 11 days.

In one embodiment, Crystalline Form 3 is kinetically stable. In one embodiment, Form 3 is kinetically stable at least through about 59% RH.

In one embodiment, Crystalline Form 3 contains some disorder in its crystallinity.

In one embodiment, the Crystalline Form 3 is substantially pure. In another embodiment, the Crystalline Form 3 is at least 95%, at least 95.5%, at least 96%, at least 96.5%, at least 97%, at least 97.5%, at least 98%, at least 98.5%, or at least 99% chemically pure (w/w %). In one embodiment, the Crystalline Form 3 is substantially pure. In another embodiment, the Crystalline Form 3 is at least about 95%, at least about 95.5%, at least about 96%, at least about 96.5%, at least about 97%, at least about 97.5%, at least about 98%, at least about 98.5%, at least about 99% or at least about 99.5% chemically pure (w/w %). In one embodiment, the Crystalline Form 3 is greater than or equal to 97.5% pure (w/w %). In one embodiment, the Crystalline Form 3 is greater than or equal to about 97.5% pure (w/w %). In one embodiment, the Crystalline Form 3 is greater than or equal to about 98.0% pure (w/w %). In one embodiment, the Crystalline Form 3 is greater than or equal to about 98.5% pure (w/w %). In one embodiment, the Crystalline Form 3 is greater than or equal to about 99.0% pure (w/w %). In one embodiment, the chemical purity is assayed by high performance liquid chromatography (HPLC).

In another embodiment, the Crystalline Form 3 comprises less than 5%, less than 4.5%, less than 4%, less than 3.5%, less than 3%, less than 2.5%, less than 2%, less than 1.5%, less than 1%, or less than 0.5% total impurities (w/w %). In another embodiment, the Crystalline Form 3 comprises less than about 5%, less than about 4.5%, less than about 4%, less than about 3.5%, less than about 3%, less than about 2.5%, less than about 2%, less than about 1.5%, less than about 1%, or less than about 0.5% total impurities (w/w %). In another embodiment, the Crystalline Form 3 comprises less than or equal to 2.5% total impurities (w/w %). In another embodiment, the Crystalline Form 3 comprises less than or equal to 1.0% total impurities (w/w %). In another embodiment, the Crystalline Form 3 comprises less than or equal to 0.5% total impurities (w/w %). In one embodiment, the impurities are measured by HPLC.

In some embodiments, the Crystalline From 3 contains about 0.5% (w/w) to about 80% Form 2. In some embodiments, the Crystalline From 3 contains about 0.5% (w/w) to about 60% Form 2. In some embodiments, the Crystalline From 3 contains about 0.5% (w/w) to about 40% Form 2.

In some embodiments, a composition comprises Crystalline Form 2 and Crystalline Form 3.

In one embodiment, the Crystalline Form 3 comprises less than 10,000 ppm, less than 7,500 ppm, less than 6,000 ppm, less than 5,000 ppm, less than 4,000 ppm, or less than 3,000 ppm acetone. In one embodiment, the Crystalline Form 3 comprises less than or equal to 5,000 ppm acetone. In some embodiments, the amount of acetone is determined by headspace gas chromatography (HS-GC).

In one embodiment, the Crystalline Form 3 comprises less than 10,000 ppm, less than 7,500 ppm, less than 6,000 ppm, less than 5,000 ppm, less than 4,000 ppm, or less than 3,000 ppm 2-propanol. In one embodiment, the Crystalline Form 3 comprises less than or equal to 5,000 ppm 2-propanol. In some embodiments, the amount of 2-propanol is determined by headspace gas chromatography (HS-GC).

In one embodiment, the Crystalline Form 3 comprises less than 1,500 ppm, less than 1,250 ppm, less than 1,000 ppm, less than 900 ppm, less than 800 ppm, less than 700 ppm or less than 600 ppm tetrahydrofuran. In one embodiment, the Crystalline Form 3 comprises less than or equal to 720 ppm tetrahydrofuran. In some embodiments, the amount of tetrahydrofuran is determined by headspace gas chromatography (HS-GC).

In one embodiment, the Crystalline Form 3 comprises less than 10,000 ppm, less than 7,500 ppm, less than 6,000 ppm, less than 5,000 ppm, less than 4,000 ppm, or less than 3,000 ppm acetic acid. In one embodiment, the Crystalline Form 3 comprises less than or equal to 5,000 ppm acetic acid. In some embodiments, the amount of acetic acid is determined by ion chromatography.

In one embodiment, the present disclosure relates to Compound I hydrate. In one embodiment, the present disclosure relates to Compound I dihydrate.

### Crystalline Compound I Form 4 (Compound I Free Base)

In one embodiment, crystalline Compound I Form 4 (Crystalline Form 4) is a hydrate. In one embodiment, Crystalline Form 4 is anhydrous.

In one embodiment, Crystalline Form 4 of Compound I-hydrate exhibits an XRPD comprising one or more peaks at about 6.6, about 10.0 and about 13.6 degrees two-theta with the margin of error of about ±0.5; about ±0.4; about ±0.3; about ±0.2; about ±0.1; about ±0.05; or less. In one embodiment, the Crystalline Form 4 exhibits an XRPD that is substantially similar to FIG. 19A (top spectrum) excluding peaks attributable to Crystalline Form 4. In one embodiment, the Crystalline Form 4 exhibits an XRPD that is substantially similar to FIG. 19A (second from top spectrum) excluding peaks attributable to Crystalline Forms 3 and 6. In one embodiment, the Crystalline Form 4 exhibits an XRPD that is substantially similar to FIG. 19B.

In one embodiment, the Crystalline Form 4 exhibits an XRPD pattern comprising peaks at 6.6±0.2, 10.0±0.2 and 13.6±0.2 degrees two-theta.

In one embodiment, the Crystalline Form 4 of Compound I exhibits an XRPD spectrum comprising peaks shown in Table C.

TABLE C
XRPD data for the Crystalline Form 4

| °2θ | d space (Å) | Intensity (%) |
|---|---|---|
| 6.61 ± 0.20 | 13.369 ± 0.404 | 79 |
| 9.97 ± 0.20 | 8.864 ± 0.177 | 100 |
| 13.61 ± 0.20 | 6.499 ± 0.095 | 71 |

In one embodiment, Crystalline Form 4 contains some disorder in its crystallinity.

In one embodiment, the Crystalline Form 4 is substantially pure. In another embodiment, the Crystalline Form 4 is at least 95%, at least 95.5%, at least 96%, at least 96.5%, at least 97%, at least 97.5%, at least 98%, at least 98.5%, or at least 99% chemically pure (w/w %). In one embodiment, the Crystalline Form 4 is substantially pure. In another embodiment, the Crystalline Form 4 is at least about 95%, at least about 95.5%, at least about 96%, at least about 96.5%, at least about 97%, at least about 97.5%, at least about 98%, at least about 98.5%, at least about 99% or at least about 99.5% chemically pure (w/w %). In one embodiment, the Crystalline Form 4 is greater than or equal to 97.5% pure (w/w %). In one embodiment, the Crystalline Form 4 is greater than or equal to about 97.5% pure (w/w %). In one embodiment, the Crystalline Form 4 is greater than or equal to about 98.0% pure (w/w %). In one embodiment, the Crystalline Form 4 is greater than or equal to about 98.5% pure (w/w %). In one embodiment, the Crystalline Form 4 is greater than or equal to about 99.0% pure (w/w %). In one embodiment, the chemical purity is assayed by high performance liquid chromatography (HPLC).

In another embodiment, the Crystalline Form 4 comprises less than 5%, less than 4.5%, less than 4%, less than 3.5%, less than 3%, less than 2.5%, less than 2%, less than 1.5%, less than 1%, or less than 0.5% total impurities (w/w %). In another embodiment, the Crystalline Form 4 comprises less than about 5%, less than about 4.5%, less than about 4%, less than about 3.5%, less than about 3%, less than about 2.5%, less than about 2%, less than about 1.5%, less than about 1%, or less than about 0.5% total impurities (w/w %). In another embodiment, the Crystalline Form 4 comprises less than or equal to 2.5% total impurities (w/w %). In another embodiment, the Crystalline Form 4 comprises less than or equal to 1.0% total impurities (w/w %). In another embodiment, the Crystalline Form 4 comprises less than or equal to 0.5% total impurities (w/w %). In one embodiment, the impurities are measured by HPLC.

In some embodiments, the Crystalline From 4 contains about 0.5% (w/w) to about 80% Form 3. In some embodiments, the Crystalline From 4 contains about 0.5% (w/w) to about 60% Form 3. In some embodiments, the Crystalline From 4 contains about 0.5% (w/w) to about 40% Form 3.

In some embodiments, the Crystalline From 4 contains about 0.5% (w/w) to about 80% Form 6. In some embodiments, the Crystalline From 4 contains about 0.5% (w/w) to about 60% Form 6. In some embodiments, the Crystalline From 4 contains about 0.5% (w/w) to about 40% Form 6.

In some embodiments, a composition comprises Crystalline Form 4 and Crystalline Form 3. In some embodiments, a composition comprises Crystalline Form 4 and Crystalline Form 6. In some embodiments, a composition comprises Crystalline Form 4, Crystalline Form 3, and Crystalline Form 6.

In some embodiments, a composition comprises Crystalline Form 2 and Crystalline Form 4. In some embodiments, a composition comprises Crystalline Form 2, Crystalline Form 3, and Crystalline Form 4. In some embodiments, a composition comprises Crystalline Form 2, Crystalline Form 3, Crystalline Form 4, and Crystalline Form 6.

In one embodiment, the present disclosure relates to anhydrous Compound I.

### Crystalline Compound I-Solvate Form 5

In one embodiment, crystalline Compound I Form 5 (Crystalline Form 5) is a solvate. In one embodiment, Crystalline Form 5 is a butanol solvate.

In one embodiment, Crystalline Form 5 comprises Compound I and butanol in about 1:1 mole ratio.

In one embodiment, Crystalline Form 5 of Compound I-hydrate exhibits an XRPD comprising one or more peaks at about 14.5 and about 21.0 degrees two-theta with the margin of error of about ±0.5; about ±0.4; about ±0.3; about ±0.2; about ±0.1; about ±0.05; or less. In another embodiment, the XRPD of the Crystalline Form 5 further comprises one or more peaks at about 9.1 and about 16.7 degrees two-theta with the margin of error of about ±0.5; about ±0.4; about ±0.3; about ±0.2; about ±0.1; about ±0.05; or less. In further embodiment, the Crystalline Form 5 further comprises one or more peaks at about 14.8, about 15.7, and about 24.2 degrees two-theta with the margin of error of about ±0.5; about ±0.4; about ±0.3; about ±0.2; about ±0.1; about ±0.05; or less. In one specific embodiment, the Crystalline Form 5 exhibits an XRPD that is substantially similar to FIG. 20.

In one embodiment, the Crystalline Form 5 exhibits an XRPD pattern comprising peaks at 14.5±0.2 and 21.0±0.2 degrees two-theta. In one embodiment, the Crystalline Form 5 exhibits an XRPD pattern comprising peaks at 9.1±0.2, 14.5±0.2, 16.7±0.2, and 21.0±0.2 degrees two-theta. In one embodiment, the Crystalline Form 5 exhibits an XRPD pattern comprising peaks at 9.1±0.2, 14.5±0.2, 14.8±0.2, 16.7±0.2, and 21.0±0.2 degrees two-theta. In one embodiment, the Crystalline Form 5 exhibits an XRPD pattern comprising peaks at 9.1±0.2, 14.5±0.2, 14.8±0.2, 15.7±0.2, 16.7±0.2, and 21.0±0.2 degrees two-theta. In one embodiment, the Crystalline Form 5 exhibits an XRPD pattern comprising peaks at 9.1±0.2, 14.5±0.2, 14.8±0.2, 15.7±0.2, 16.7±0.2, 21.0±0.2, and 24.2±0.2 degrees two-theta.

In one embodiment, the Crystalline Form 5 of Compound I exhibits an XRPD spectrum comprising peaks shown in Table D1. In one embodiment, the Crystalline Form 5 exhibits an XRPD spectrum comprising peaks shown in Table D2. In one embodiment, the Crystalline Form 5 exhibits an XRPD spectrum comprising all peaks in Table D1 having at least 40% intensity, with the understanding that some of the close peaks can form one broad peak. In one embodiment, the Crystalline Form 5 exhibits an XRPD spectrum comprising all peaks in Table D2 having at least 40% intensity, with the understanding that some of the close peaks can form one broad peak. In one embodiment, the Crystalline Form 5 exhibits an XRPD spectrum comprising all peaks in Table D2 having at least 20% intensity, with the understanding that some of the close peaks can form one broad peak. In one embodiment, the Crystalline Form 5 exhibits an XRPD spectrum comprising all peaks in Table D2 having at least 15% intensity, with the understanding that some of the close peaks can form one broad peak.

TABLE D1
XRPD data for the Crystalline Form 5

| °2θ | d space (Å) | Intensity (%) |
|---|---|---|
| 5.93 ± 0.20 | 14.892 ± 0.502 | 2 |
| 9.05 ± 0.20 | 9.764 ± 0.215 | 20 |
| 10.41 ± 0.20 | 8.491 ± 0.163 | 6 |
| 12.04 ± 0.20 | 7.345 ± 0.122 | 7 |
| 12.36 ± 0.20 | 7.155 ± 0.115 | 6 |
| 13.10 ± 0.20 | 6.753 ± 0.103 | 2 |
| 14.48 ± 0.20 | 6.112 ± 0.084 | 48 |
| 14.79 ± 0.20 | 5.985 ± 0.080 | 19 |
| 15.67 ± 0.20 | 5.651 ± 0.072 | 19 |
| 16.70 ± 0.20 | 5.304 ± 0.063 | 24 |
| 17.04 ± 0.20 | 5.199 ± 0.061 | 6 |
| 17.16 ± 0.20 | 5.163 ± 0.060 | 5 |
| 17.42 ± 0.20 | 5.087 ± 0.058 | 5 |
| 17.89 ± 0.20 | 4.954 ± 0.055 | 3 |
| 18.79 ± 0.20 | 4.719 ± 0.050 | 8 |
| 18.96 ± 0.20 | 4.677 ± 0.049 | 5 |
| 19.37 ± 0.20 | 4.579 ± 0.047 | 1 |
| 20.82 ± 0.20 | 4.263 ± 0.041 | 10 |
| 21.02 ± 0.20 | 4.223 ± 0.040 | 100 |
| 21.41 ± 0.20 | 4.147 ± 0.038 | 9 |
| 21.65 ± 0.20 | 4.101 ± 0.037 | 10 |
| 22.67 ± 0.20 | 3.919 ± 0.034 | 14 |
| 23.10 ± 0.20 | 3.847 ± 0.033 | 5 |
| 23.47 ± 0.20 | 3.787 ± 0.032 | 7 |
| 24.01 ± 0.20 | 3.703 ± 0.030 | 4 |
| 24.23 ± 0.20 | 3.670 ± 0.030 | 19 |
| 24.89 ± 0.20 | 3.574 ± 0.028 | 3 |
| 25.16 ± 0.20 | 3.537 ± 0.028 | 11 |
| 25.72 ± 0.20 | 3.461 ± 0.026 | 7 |
| 26.39 ± 0.20 | 3.375 ± 0.025 | 2 |
| 26.61 ± 0.20 | 3.347 ± 0.025 | 4 |
| 27.90 ± 0.20 | 3.195 ± 0.022 | 5 |
| 28.46 ± 0.20 | 3.134 ± 0.022 | 3 |
| 29.10 ± 0.20 | 3.066 ± 0.021 | 6 |
| 29.37 ± 0.20 | 3.039 ± 0.020 | 6 |
| 29.62 ± 0.20 | 3.014 ± 0.020 | 1 |
| 29.98 ± 0.20 | 2.978 ± 0.019 | 2 |
| 30.61 ± 0.20 | 2.918 ± 0.019 | 2 |
| 30.73 ± 0.20 | 2.907 ± 0.018 | 2 |
| 30.94 ± 0.20 | 2.888 ± 0.018 | 1 |
| 31.29 ± 0.20 | 2.856 ± 0.018 | 2 |
| 31.63 ± 0.20 | 2.826 ± 0.017 | 4 |

TABLE D2
XRPD data for the Crystalline Form 5

| °2θ | d space (Å) | Intensity (%) |
|---|---|---|
| 9.05 ± 0.20 | 9.764 ± 0.215 | 20 |
| 14.48 ± 0.20 | 6.112 ± 0.084 | 48 |
| 14.79 ± 0.20 | 5.985 ± 0.080 | 19 |
| 15.67 ± 0.20 | 5.651 ± 0.072 | 19 |
| 16.70 ± 0.20 | 5.304 ± 0.063 | 24 |
| 21.02 ± 0.20 | 4.223 ± 0.040 | 100 |
| 22.67 ± 0.20 | 3.919 ± 0.034 | 14 |
| 24.23 ± 0.20 | 3.670 ± 0.030 | 19 |

In one embodiment, the Crystalline Form 5 exhibits a DSC thermogram comprising at least one small endotherm between about 130° C. to about 170° C. In one embodiment the Crystalline Form 5 exhibits a DSC thermogram comprising an endotherm peak (minimum) in between 140° C. and about 160° C. with the error of margin of about ±2.5; about ±2.0; about ±1.5; about ±1.0; about ±0.5; or less. In one embodiment the Crystalline Form 5 exhibits a DSC thermogram comprising an endotherm peak in between 140° C.±1° C. to about 160° C.±1° C. In one embodiment the Crystalline Form 5 exhibits a DSC thermogram comprising an endotherm peak in between 150° C.±1° C. to about 160° C.±1° C.

In one embodiment, the Crystalline Form 5 exhibits a DSC thermogram comprising at least one endotherm between about 170° C. to about 200° C. In one embodiment the Crystalline Form 5 exhibits a DSC thermogram comprising an endotherm peak (minimum) in between 170° C. and about 190° C. with the error of margin of about ±2.5; about ±2.0; about ±1.5; about ±1.0; about ±0.5; or less. In one embodiment the Crystalline Form 5 exhibits a DSC thermogram comprising an endotherm peak in between 147° C.±1° C. to about 190° C.±1° C. In one embodiment the Crystalline Form 5 exhibits a DSC thermogram comprising an endotherm peak in between 170° C.±1° C. to about 185° C.±1° C.

In one specific embodiment, the Crystalline Form 5 exhibits a DSC thermogram that is substantially similar to FIG. 21.

In one embodiment, the Crystalline Form 5 exhibits a TGA thermogram that is substantially similar to FIG. 21. In other embodiments, the TGA thermogram of the Crystalline Form 5 exhibits a weight loss of about 0.0 to about 25% in the temperature range of 25° C. to 250° C. In other embodiments, the TGA thermogram of the Crystalline Form 5 exhibits a weight loss of about 10% to about 25% in the temperature range of 100° C. to 250° C.

In other embodiments, the TGA thermogram of the Crystalline Form 5 exhibits a weight loss of about 15% to about 20% in the temperature range of 110° C. to 210° C. In other embodiments, the TGA thermogram of the Crystalline Form 5 exhibits a weight loss of about 17 wt %.

In one embodiment, the Crystalline Form 5 is substantially pure. In another embodiment, the Crystalline Form 5 is at least 95%, at least 95.5%, at least 96%, at least 96.5%, at least 97%, at least 97.5%, at least 98%, at least 98.5%, or at least 99% chemically pure (w/w %). In one embodiment, the Crystalline Form 5 is substantially pure. In another embodiment, the Crystalline Form 5 is at least about 95%, at least about 95.5%, at least about 96%, at least about 96.5%, at least about 97%, at least about 97.5%, at least about 98%, at least about 98.5%, at least about 99% or at least about 99.5% chemically pure (w/w %). In one embodiment, the Crystalline Form 5 is greater than or equal to 97.5% pure (w/w %). In one embodiment, the Crystalline Form 5 is greater than or equal to about 97.5% pure (w/w %). In one embodiment, the Crystalline Form 5 is greater than or equal to about 98.0% pure (w/w %). In one embodiment, the Crystalline Form 5 is greater than or equal to about 98.5% pure (w/w %). In one embodiment, the Crystalline Form 5 is greater than or equal to about 99.0% pure (w/w %). In one embodiment, the chemical purity is assayed by high performance liquid chromatography (HPLC).

In another embodiment, the Crystalline Form 5 comprises less than 5%, less than 4.5%, less than 4%, less than 3.5%, less than 3%, less than 2.5%, less than 2%, less than 1.5%, less than 1%, or less than 0.5% total impurities (w/w %). In another embodiment, the Crystalline Form 5 comprises less than about 5%, less than about 4.5%, less than about 4%, less than about 3.5%, less than about 3%, less than about 2.5%, less than about 2%, less than about 1.5%, less than about 1%, or less than about 0.5% total impurities (w/w %). In another embodiment, the Crystalline Form 5 comprises less than or equal to 2.5% total impurities (w/w %). In another embodiment, the Crystalline Form 5 comprises less than or equal to 1.0% total impurities (w/w %). In another embodiment, the Crystalline Form 5 comprises less than or equal to 0.5% total impurities (w/w %). In one embodiment, the impurities are measured by HPLC.

In one embodiment, the present disclosure relates to Compound I butanol solvate.

### Crystalline Compound I Form 6 (Anhydrous)

In one embodiment, crystalline Compound I Form 6 (Crystalline Form 6) is anhydrous.

In one embodiment, Crystalline Form 6 of Compound I-hydrate exhibits an XRPD comprising one or more peaks at about 9.1, about 15.1, and about 25.3 degrees two-theta with the margin of error of about ±0.5; about ±0.4; about ±0.3; about ±0.2; about ±0.1; about ±0.05; or less. In another embodiment, the XRPD of the Crystalline Form 6 further comprises one or more peaks at about 14.7 and about 14.8 degrees two-theta with the margin of error of about ±0.5; about ±0.4; about ±0.3; about ±0.2; about ±0.1; about ±0.05; or less. In further embodiment, the Crystalline Form 6 further comprises one or more peaks at about 26.4 degrees two-theta with the margin of error of about ±0.5; about ±0.4; about ±0.3; about ±0.2; about ±0.1; about ±0.05; or less. In one specific embodiment, the Crystalline Form 6 exhibits an XRPD that is substantially similar to FIG. 22.

In one embodiment, the Crystalline Form 6 exhibits an XRPD pattern comprising peaks at 9.1±0.2, 15.1±0.2, and 25.3±0.2 degrees two-theta. In one embodiment, the Crystalline Form 6 exhibits an XRPD pattern comprising peaks at 9.1±0.2, 14.7±0.2, 14.8±0.2, 15.1±0.2, and 25.3±0.2 degrees two-theta. In one embodiment, the Crystalline Form 6 exhibits an XRPD pattern comprising peaks at 9.1±0.2, 14.7±0.2, 14.8±0.2, 15.1±0.2, 25.3±0.2, and 26.4±0.2 degrees two-theta. In one embodiment, the Crystalline Form 6 exhibits an XRPD pattern comprising peaks at 9.1±0.2, 14.7±0.2, 14.8±0.2, 15.1±0.2, 19.7±0.2, 25.3±0.2, and 26.4±0.2 degrees two-theta.

In one embodiment, the Crystalline Form 6 exhibits an XRPD spectrum comprising peaks shown in Table E1. In one embodiment, the Crystalline Form 6 exhibits an XRPD spectrum comprising peaks shown in Table E2. In one embodiment, the Crystalline Form 6 exhibits an XRPD spectrum comprising all peaks in Table E1 having at least 50% intensity, with the understanding that some of the close peaks can form one broad peak. In one embodiment, the Crystalline Form 6 exhibits an XRPD spectrum comprising all peaks in Table E2 having at least 50% intensity, with the understanding that some of the close peaks can form one broad peak. In one embodiment, the Crystalline Form 6 exhibits an XRPD spectrum comprising all peaks in Table E2 having at least 40% intensity, with the understanding that some of the close peaks can form one broad peak. In one embodiment, the Crystalline Form 6 exhibits an XRPD spectrum comprising all peaks in Table E2 having at least 30% intensity, with the understanding that some of the close peaks can form one broad peak.

TABLE E1
XRPD data for the Crystalline Form 6

| °2θ | d space (Å) | Intensity (%) |
|---|---|---|
| 9.06 ± 0.20 | 9.753 ± 0.215 | 73 |
| 11.79 ± 0.20 | 7.500 ± 0.127 | 7 |
| 12.58 ± 0.20 | 7.033 ± 0.111 | 3 |
| 13.92 ± 0.20 | 6.357 ± 0.091 | 5 |
| 14.67 ± 0.20 | 6.033 ± 0.082 | 42 |
| 14.83 ± 0.20 | 5.969 ± 0.080 | 52 |
| 15.08 ± 0.20 | 5.870 ± 0.077 | 93 |
| 16.67 ± 0.20 | 5.314 ± 0.063 | 9 |
| 17.63 ± 0.20 | 5.027 ± 0.057 | 5 |
| 18.02 ± 0.20 | 4.919 ± 0.054 | 24 |
| 19.70 ± 0.20 | 4.503 ± 0.045 | 31 |
| 21.24 ± 0.20 | 4.180 ± 0.039 | 8 |
| 23.60 ± 0.20 | 3.767 ± 0.031 | 11 |
| 24.03 ± 0.20 | 3.700 ± 0.030 | 4 |
| 25.32 ± 0.20 | 3.515 ± 0.027 | 100 |
| 26.44 ± 0.20 | 3.368 ± 0.025 | 36 |
| 27.34 ± 0.20 | 3.259 ± 0.023 | 14 |
| 28.38 ± 0.20 | 3.142 ± 0.022 | 14 |

TABLE E2
XRPD data for the Crystalline Form 6

| °2θ | d space (Å) | Intensity (%) |
|---|---|---|
| 9.06 ± 0.20 | 9.753 ± 0.215 | 73 |
| 14.67 ± 0.20 | 6.033 ± 0.082 | 42 |
| 14.83 ± 0.20 | 5.969 ± 0.080 | 52 |
| 15.08 ± 0.20 | 5.870 ± 0.077 | 93 |
| 18.02 ± 0.20 | 4.919 ± 0.054 | 24 |
| 19.70 ± 0.20 | 4.503 ± 0.045 | 31 |
| 25.32 ± 0.20 | 3.515 ± 0.027 | 100 |
| 26.44 ± 0.20 | 3.368 ± 0.025 | 36 |

In one embodiment, the Crystalline Form 6 exhibits a TGA thermogram that is substantially similar to FIG. 23. In other embodiments, the TGA thermogram of the Crystalline Form 6 exhibits a weight loss of about 0.0 to about 5% in the temperature range of 25° C. to 250° C. In other embodiments, the TGA thermogram of the Crystalline Form 6 exhibits a weight loss of about 0% to about 3% in the temperature range of 30° C. to 200° C. In other embodiments, the TGA thermogram of the Crystalline Form 6 exhibits a weight loss of about 0% to about 2% in the temperature range of 30° C. to 190° C.

In one embodiment, Crystalline Form 6 transforms to Crystalline Form 2 when exposed to about 75% RH for about 11 days.

In one embodiment, the Crystalline Form 6 is kinetically stable.

In one embodiment, the Crystalline Form 6 is substantially pure. In another embodiment, the Crystalline Form 5 is at least 95%, at least 95.5%, at least 96%, at least 96.5%, at least 97%, at least 97.5%, at least 98%, at least 98.5%, or at least 99% chemically pure (w/w %). In one embodiment, the Crystalline Form 6 is substantially pure. In another embodiment, the Crystalline Form 6 is at least about 95%, at least about 95.5%, at least about 96%, at least about 96.5%, at least about 97%, at least about 97.5%, at least about 98%, at least about 98.5%, at least about 99% or at least about 99.5% chemically pure (w/w %). In one embodiment, the Crystalline Form 6 is greater than or equal to 97.5% pure (w/w %). In one embodiment, the Crystalline Form 6 is greater than or equal to about 97.5% pure (w/w %). In one embodiment, the Crystalline Form 6 is greater than or equal to about 98.0% pure (w/w %). In one embodiment, the Crystalline Form 6 is greater than or equal to about 98.5% pure (w/w %). In one embodiment, the Crystalline Form 6 is greater than or equal to about 99.0% pure (w/w %). In one embodiment, the chemical purity is assayed by high performance liquid chromatography (HPLC).

In another embodiment, the Crystalline Form 6 comprises less than 5%, less than 4.5%, less than 4%, less than 3.5%, less than 3%, less than 2.5%, less than 2%, less than 1.5%, less than 1%, or less than 0.5% total impurities (w/w %). In another embodiment, the Crystalline Form 6 comprises less than about 5%, less than about 4.5%, less than about 4%, less than about 3.5%, less than about 3%, less than about 2.5%, less than about 2%, less than about 1.5%, less than about 1%, or less than about 0.5% total impurities (w/w %). In another embodiment, the Crystalline Form 6 comprises less than or equal to 2.5% total impurities (w/w %). In another embodiment, the Crystalline Form 6 comprises less than or equal to 1.0% total impurities (w/w %). In another embodiment, the Crystalline Form 6 comprises less than or equal to 0.5% total impurities (w/w %). In one embodiment, the impurities are measured by HPLC.

In some embodiments, the Crystalline From 6 contains about 0.5% (w/w) to about 80% Form 2. In some embodiments, the Crystalline From 6 contains about 0.5% (w/w) to about 60% Form 2. In some embodiments, the Crystalline From 6 contains about 0.5% (w/w) to about 40% Form 2.

In some embodiments, a composition comprises Crystalline Form 2 and Crystalline Form 6.

In one embodiment, the Crystalline Form 6 comprises less than 10,000 ppm, less than 7,500 ppm, less than 6,000 ppm, less than 5,000 ppm, less than 4,000 ppm, or less than 3,000 ppm acetone. In one embodiment, the Crystalline Form 6 comprises less than or equal to 5,000 ppm acetone. In some embodiments, the amount of acetone is determined by headspace gas chromatography (HS-GC).

In one embodiment, the Crystalline Form 6 comprises less than 10,000 ppm, less than 7,500 ppm, less than 6,000 ppm, less than 5,000 ppm, less than 4,000 ppm, or less than 3,000 ppm 2-propanol. In one embodiment, the Crystalline Form 6 comprises less than or equal to 5,000 ppm 2-propanol. In some embodiments, the amount of 2-propanol is determined by headspace gas chromatography (HS-GC).

In one embodiment, the Crystalline Form 6 comprises less than 1,500 ppm, less than 1,250 ppm, less than 1,000 ppm, less than 900 ppm, less than 800 ppm, less than 700 ppm or less than 600 ppm tetrahydrofuran. In one embodiment, the Crystalline Form 6 comprises less than or equal to 720 ppm tetrahydrofuran. In some embodiments, the amount of tetrahydrofuran is determined by headspace gas chromatography (HS-GC).

In one embodiment, the Crystalline Form 6 comprises less than 10,000 ppm, less than 7,500 ppm, less than 6,000 ppm, less than 5,000 ppm, less than 4,000 ppm, or less than 3,000 ppm acetic acid. In one embodiment, the Crystalline Form 6 comprises less than or equal to 5,000 ppm acetic acid. In some embodiments, the amount of acetic acid is determined by ion chromatography.

In one embodiment, the present disclosure relates to anhydrous Compound I.

### Crystalline Compound I-Solvate Form 7

In one embodiment, crystalline Compound I Form 7 (Crystalline Form 7) is a solvate. In one embodiment, Crystalline Form 7 is an isopropanol solvate.

In one embodiment, the Crystalline Form 7 exhibits an XRPD spectrum comprising peaks shown in Table F. In one embodiment, the Crystalline Form 7 exhibits an XRPD that is substantially similar to FIG. 24 (top line).

TABLE F
XRPD data for the Crystalline Form 7

| °2θ | d space (Å) | Intensity (%) |
|---|---|---|
| 9.23 ± 0.20 | 9.574 ± 0.207 | 33 |
| 15.03 ± 0.20 | 5.890 ± 0.078 | 54 |
| 15.31 ± 0.20 | 5.783 ± 0.075 | 37 |
| 19.12 ± 0.20 | 4.638 ± 0.048 | 54 |
| 21.64 ± 0.20 | 4.103 ± 0.037 | 100 |
| 23.24 ± 0.20 | 3.824 ± 0.032 | 20 |
| 24.72 ± 0.20 | 3.599 ± 0.029 | 25 |

Crystalline Form 7 was prepared by a slurry of Compound I (free base) in isopropanol and stirring at room temperature or at 50° C. for about 2.5 weeks.

### Crystalline Compound I-Solvate Form 8

In one embodiment, crystalline Compound I Form 8 (Crystalline Form 8) is a solvate. In one embodiment, Crystalline Form 8 is a methanol solvate.

In one embodiment, the Crystalline Form 8 exhibits an XRPD spectrum comprising peaks shown in Table G. In one embodiment, the Crystalline Form 8 exhibits an XRPD that is substantially similar to FIG. 24 (second from top line).

TABLE G
XRPD data for the Crystalline Form 8

| °2θ | d space (Å) | Intensity (%) |
|---|---|---|
| 7.65 ± 0.20 | 11.547 ± 0.301 | 21 |
| 9.04 ± 0.20 | 9.774 ± 0.216 | 100 |
| 10.96 ± 0.20 | 8.066 ± 0.147 | 23 |
| 14.01 ± 0.20 | 6.316 ± 0.090 | 14 |
| 23.99 ± 0.20 | 3.706 ± 0.030 | 35 |
| 24.81 ± 0.20 | 3.586 ± 0.028 | 18 |
| 25.29 ± 0.20 | 3.519 ± 0.027 | 38 |
| 26.06 ± 0.20 | 3.417 ± 0.026 | 63 |
| 28.02 ± 0.20 | 3.182 ± 0.022 | 20 |

Crystalline Form 8 was prepared by a slurry of Compound I (free base) in methanol and stirring at room temperature for about 2.5 weeks.

### Crystalline Compound I-Solvate Form 9

In one embodiment, crystalline Compound I Form 9 (Crystalline Form 9) is a solvate. In one embodiment, Crystalline Form 9 is a tetrahydrofuran solvate.

In one embodiment, the Crystalline Form 9 exhibits an XRPD spectrum comprising peaks shown in Table H. In one embodiment, the Crystalline Form 9 exhibits an XRPD that is substantially similar to FIG. 24 (third from top line).

TABLE H
XRPD data for the Crystalline Form 9

| °2θ | d space (Å) | Intensity (%) |
|---|---|---|
| 7.07 ± 0.20 | 12.493 ± 0.353 | 84 |
| 13.99 ± 0.20 | 6.325 ± 0.090 | 53 |
| 14.43 ± 0.20 | 6.133 ± 0.085 | 30 |
| 15.04 ± 0.20 | 5.886 ± 0.078 | 91 |
| 15.70 ± 0.20 | 5.640 ± 0.071 | 29 |
| 16.65 ± 0.20 | 5.320 ± 0.063 | 31 |
| 23.99 ± 0.20 | 3.706 ± 0.030 | 32 |
| 24.87 ± 0.20 | 3.577 ± 0.028 | 56 |
| 25.20 ± 0.20 | 3.531 ± 0.028 | 49 |
| 26.02 ± 0.20 | 3.422 ± 0.026 | 100 |
| 27.20 ± 0.20 | 3.276 ± 0.024 | 30 |

Crystalline Form 9 was prepared by a slurry of Compound I (free base) in tetrahydrofuran and stirring at room temperature for about 2.5 weeks.

### Crystalline Compound I-Solvate Form 10

In one embodiment, crystalline Compound I Form 10 (Crystalline Form 10) is a solvate. In one embodiment, Crystalline Form 10 is a N-methyl-2-pyrrolidone (NMP) solvate.

In one embodiment, the Crystalline Form 10 exhibits an XRPD spectrum comprising peaks shown in Table I. In one embodiment, the Crystalline Form 10 exhibits an XRPD that is substantially similar to FIG. 24 (fourth from top line).

TABLE I
XRPD data for the Crystalline Form 10

| °2θ | d space (Å) | Intensity (%) |
|---|---|---|
| 5.53 ± 0.20 | 15.968 ± 0.577 | 33 |
| 6.75 ± 0.20 | 13.085 ± 0.387 | 23 |
| 11.63 ± 0.20 | 7.603 ± 0.130 | 26 |
| 12.89 ± 0.20 | 6.862 ± 0.106 | 39 |
| 13.53 ± 0.20 | 6.539 ± 0.096 | 89 |
| 15.48 ± 0.20 | 5.720 ± 0.073 | 89 |
| 16.93 ± 0.20 | 5.233 ± 0.061 | 30 |
| 17.12 ± 0.20 | 5.175 ± 0.060 | 24 |
| 24.33 ± 0.20 | 3.655 ± 0.030 | 37 |
| 24.88 ± 0.20 | 3.576 ± 0.028 | 100 |
| 25.65 ± 0.20 | 3.470 ± 0.027 | 78 |
| 25.97 ± 0.20 | 3.428 ± 0.026 | 34 |
| 26.26 ± 0.20 | 3.391 ± 0.025 | 86 |
| 26.73 ± 0.20 | 3.332 ± 0.024 | 26 |

Crystalline Form 10 was prepared by a slurry of Compound I (free base) in NMP and stirring at room temperature for about 2.5 weeks.

### Crystalline Compound I-Solvate Form 11

In one embodiment, crystalline Compound I Form 11 (Crystalline Form 11) is a solvate. In one embodiment, Crystalline Form 11 is a hexafluoroisopropanol solvate.

In one embodiment, the Crystalline Form 11 exhibits an XRPD spectrum comprising peaks shown in Table J. In one embodiment, the Crystalline Form 11 exhibits an XRPD that is substantially similar to FIG. 24 (fifth from top line).

TABLE J
XRPD data for the Crystalline Form 11

| °2θ | d space (Å) | Intensity (%) |
|---|---|---|
| 9.31 ± 0.20 | 9.492 ± 0.203 | 57 |
| 14.42 ± 0.20 | 6.138 ± 0.085 | 100 |
| 16.48 ± 0.20 | 5.375 ± 0.065 | 63 |
| 16.69 ± 0.20 | 5.308 ± 0.063 | 51 |
| 16.87 ± 0.20 | 5.251 ± 0.062 | 73 |
| 17.33 ± 0.20 | 5.113 ± 0.059 | 76 |
| 17.84 ± 0.20 | 4.968 ± 0.055 | 95 |
| 17.96 ± 0.20 | 4.935 ± 0.055 | 64 |
| 18.26 ± 0.20 | 4.855 ± 0.053 | 90 |
| 18.89 ± 0.20 | 4.694 ± 0.049 | 57 |
| 19.34 ± 0.20 | 4.586 ± 0.047 | 66 |
| 19.92 ± 0.20 | 4.454 ± 0.044 | 64 |
| 20.17 ± 0.20 | 4.399 ± 0.043 | 52 |
| 21.33 ± 0.20 | 4.162 ± 0.039 | 72 |
| 22.81 ± 0.20 | 3.895 ± 0.234 | 47 |
| 23.22 ± 0.20 | 3.828 ± 0.033 | 44 |

Crystalline Form 11 was prepared by a slurry of Compound I (free base) in hexafluoroisopropanol (HFIPA)/water (97/3) and stirring at room temperature for about 2.5 weeks.

### Crystalline Compound I-Solvate Form 12

In one embodiment, crystalline Compound I Form 12 (Crystalline Form 12) is a solvate. In one embodiment, Crystalline Form 12 is a hexafluoroisopropanol solvate.

In one embodiment, the Crystalline Form 12 exhibits an XRPD spectrum comprising peaks shown in Table K. In one embodiment, the Crystalline Form 12 exhibits an XRPD that is substantially similar to FIG. 24 (sixth from top line).

TABLE K
XRPD data for the Crystalline Form 12

| °2θ | d space (Å) | Intensity (%) |
|---|---|---|
| 5.53 ± 0.20 | 15.968 ± 0.577 | 38 |
| 9.12 ± 0.20 | 9.689 ± 0.212 | 54 |
| 9.48 ± 0.20 | 9.322 ± 0.196 | 48 |
| 9.59 ± 0.20 | 9.215 ± 0.192 | 47 |
| 12.31 ± 0.20 | 7.184 ± 0.116 | 28 |
| 14.38 ± 0.20 | 6.154 ± 0.085 | 37 |
| 14.61 ± 0.20 | 6.058 ± 0.082 | 70 |
| 15.42 ± 0.20 | 5.742 ± 0.074 | 38 |
| 16.06 ± 0.20 | 5.514 ± 0.068 | 100 |
| 16.40 ± 0.20 | 5.401 ± 0.065 | 51 |
| 17.47 ± 0.20 | 5.072 ± 0.058 | 90 |
| 18.38 ± 0.20 | 4.823 ± 0.052 | 62 |
| 19.27 ± 0.20 | 4.602 ± 0.047 | 53 |
| 19.56 ± 0.20 | 4.535 ± 0.046 | 62 |
| 21.37 ± 0.20 | 4.155 ± 0.038 | 40 |
| 22.19 ± 0.20 | 4.003 ± 0.036 | 36 |
| 22.94 ± 0.20 | 3.874 ± 0.033 | 50 |

Crystalline Form 12 was prepared by a slurry of Compound I (free base) in hexafluoroisopropanol (HFIPA)/water (98/2) and stirring at room temperature for about 2.5 weeks.

### Crystalline Compound I Form 13

In one embodiment, the crystalline form is crystalline Compound I Form 13 (Crystalline Form 13).

In one embodiment, the Crystalline Form 13 exhibits an XRPD spectrum comprising peaks shown in Table L. In one embodiment, the Crystalline Form 13 exhibits an XRPD that is substantially similar to FIG. 25, excluding peaks attributable to Form 6.

TABLE L
XRPD data for the Crystalline Form 13

| °2θ | d space (Å) | Intensity (%) |
|---|---|---|
| 7.29 ± 0.20 | 12.124 ± 0.332 | 100 |
| 9.52 ± 0.20 | 9.283 ± 0.195 | 91 |
| 13.57 ± 0.20 | 6.519 ± 0.096 | 83 |
| 14.45 ± 0.20 | 6.126 ± 0.084 | 91 |
| 16.20 ± 0.20 | 5.466 ± 0.067 | 73 |
| 18.60 ± 0.20 | 4.766 ± 0.051 | 68 |
| 21.41 ± 0.20 | 4.146 ± 0.038 | 51 |
| 23.82 ± 0.20 | 3.732 ± 0.031 | 61 |

Crystalline Form 13 was prepared by drying Form 5 at 220° C. for 1 day.

### Crystalline Compound I-Solvate Form 14

In one embodiment, crystalline Compound I Form 14 (Crystalline Form 14) is a solvate. In one embodiment, Crystalline Form 14 is an NMP solvate.

In one embodiment, the Crystalline Form 14 exhibits an XRPD spectrum comprising peaks shown in Table M. In one embodiment, the Crystalline Form 14 exhibits an XRPD that is substantially similar to FIG. 24 (bottom line).

TABLE M
XRPD data for the Crystalline Form 14

| °2θ | d space (Å) | Intensity (%) |
|---|---|---|
| 5.22 ± 0.20 | 16.916 ± 0.648 | 46 |
| 7.43 ± 0.20 | 11.888 ± 0.320 | 18 |
| 13.32 ± 0.20 | 6.642 ± 0.099 | 31 |
| 14.19 ± 0.20 | 6.236 ± 0.087 | 34 |
| 14.55 ± 0.20 | 6.083 ± 0.083 | 20 |
| 15.11 ± 0.20 | 5.859 ± 0.077 | 19 |
| 15.39 ± 0.20 | 5.753 ± 0.074 | 65 |
| 17.97 ± 0.20 | 4.933 ± 0.054 | 26 |
| 23.80 ± 0.20 | 3.736 ± 0.031 | 21 |
| 24.63 ± 0.20 | 3.611 ± 0.029 | 35 |
| 24.89 ± 0.20 | 3.575 ± 0.028 | 100 |
| 26.20 ± 0.20 | 3.399 ± 0.025 | 33 |
| 26.61 ± 0.20 | 3.347 ± 0.025 | 28 |

Crystalline Form 14 was prepared by a slurry of Compound I (free base) in NMP and stirring at 2-8° C. for about 2.5 weeks.

### Compound I Salts

In one embodiment, the crystalline form of Compound I is a salt. In some embodiments, the crystalline form of Compound I is a salt is an acid addition salt. In another embodiment, the crystalline form of Compound I acid addition salt where the acid is selected from hydrochloric acid (HCl), sulfuric acid, nitric acid, phosphoric acid, methanesulfonic acid, citric acid, maleic acid, succinic acid, or the like.

In one embodiment, the crystalline form of Compound I is a salt is HCl addition salt. In one embodiment, the crystalline form of Compound I is a mono-HCl salt. In another embodiment, the crystalline form of Compound I is a bis-HCl salt (Compound I-HCl).

In one embodiment, crystalline form of Compound I-HCl exhibits different polymorphs, which are but not limited to, Forms A and B, as defined in the following sections.

In one embodiment of the present disclosure, the crystalline form of Compound I may comprise of a mixture of one or more forms of polymorphs of Compound I and/or various Compound I salts and/or Compound I-HCl. In some embodiments, the crystalline form of Compound I-HCl may comprise of substantially pure form of one polymorph type. In one embodiment, the crystalline form of Compound I-HCl may comprise of over about 99.9%, about 99.8%, about 99.7%, about 99.6%, about 99.5%, about 99.4%, about 99.3%, about 99.2%, about 99.1%, or about 99.0% of one polymorph of Compound I-HCl. In another embodiment, the crystalline form of Compound I-HCl may comprise over about 99%, 98%, 97%, 96%, 95%, 94%, 93%, 92%, 91%, or 90% of one polymorph of Compound I-HCl. In some embodiments, the crystalline form of Compound I-acetate may comprise over about 90%, 85%, 80%, 75%, 70%, 65%, 60%, 55%, 50%, 45%, or 40% of one polymorph of Compound I-HCl.

In some embodiments, the crystalline form of Compound I-HCl may comprise of substantially pure form of one polymorph type. In one embodiment, the crystalline form of Compound I-HCl may comprise of over about 99.9%, about 99.8%, about 99.7%, about 99.6%, about 99.5%, about 99.4%, about 99.3%, about 99.2%, about 99.1%, or about 99.0% of one polymorph of Compound I-HCl. In another embodiment, the crystalline form of Compound I-HCl may comprise over about 99%, 98%, 97%, 96%, 95%, 94%, 93%, 92%, 91%, or 90% of one polymorph of Compound I-HCl. In some embodiments, the crystalline form of Compound I-HCl may comprise over about 90%, 85%, 80%, 75%, 70%, 65%, 60%, 55%, 50%, 45%, or 40% of one polymorph of Compound I-HCl.

In one embodiment of the present disclosure, the crystalline form of Compound I may comprise of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, about 90%, about 85%, about 80%, about 75%, about 70%, about 65%, about 60%, about 55% or about 50% of crystalline Compound I-HCl Form A.

In one embodiment of the present disclosure, the crystalline form of Compound I can be crystalline Compound I-HCl Form A comprising about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45% or 50% of crystalline Compound I-HCl Form B.

In one embodiment of the present disclosure, the crystalline form of Compound I can be crystalline Compound I-HCl Form A comprising about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45% or 50% of crystalline Compound I-HCl Form B.

In one embodiment of the present disclosure, the crystalline form of Compound I can comprise of at least about 99.9%, about 99.8%, about 99.7%, about 99.6%, about 99.5%, about 99.4%, about 99.3%, about 99.2%, about 99.1%, about 99.0%, about 98%, about 97%, about 96%, about 95%, about 94%, about 93%, about 92%, about 91%, about 90%, about 85%, about 80%, about 75%, about 70%, about 65%, about 60%, about 55% or about 50% of crystalline Compound I-HCl Form B.

In one embodiment of the present disclosure, the crystalline form of Compound I can be crystalline Compound I-HCl Form B comprising about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45% or 50% of crystalline Compound I-HCl Form A.

In one embodiment of the present disclosure, the crystalline form of Compound I can be crystalline Compound I-HCl Form B comprising about 0.1%, 0.2%, 0.3%, 0.4%, 0.5%, 0.6%, 0.7%, 0.8%, 0.9%, 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 11%, 12%, 13%, 14%, 15%, 16%, 17%, 18%, 19%, 20%, 25%, 30%, 35%, 40%, 45% or 50% of crystalline Compound I-HCl Form A.

### Crystalline Compound I HCl Salt Form A (Bis HCl Salt)

In one embodiment, the crystalline form of the Compound I-HCl is Crystalline Form A of (Crystalline Form A). In one embodiment, the Crystalline Form A exhibits an XRPD comprising one or more peaks at about 10.5 and 27 degrees two-theta with the margin of error of about ±0.5; about ±0.4; about ±0.3; about ±0.2; about ±0.1; about ±0.05; or less.

In one specific embodiment, the Crystalline Form A exhibits an XRPD that is substantially similar to FIG. 9 (top line).

In one embodiment, the Crystalline Form A exhibits a DSC thermogram comprising a broad endotherm between about 250° C. to about 310° C. In one embodiment, the Crystalline Form A exhibits a DSC thermogram comprising at least two broad endotherm peaks between about 250° C. to about 310° C. In one embodiment, the Crystalline Form A exhibits a DSC thermogram comprising an exotherm between at about 242° C. with the error of margin of about ±2.5; about ±2.0; about ±1.5; about ±1.0; about ±0.5; or less. In one specific embodiment, the Crystalline Form A exhibits a DSC thermogram that is substantially similar to FIG. 10 (top line of bottom set).

In one embodiment, the Crystalline Form A exhibits a TGA thermogram that is substantially similar to FIG. 10 (top line of top set). In other embodiments, the TGA thermogram of the Crystalline Form A exhibits a weight loss of about 0.0 to about 12% in the temperature range of 25° C. to 250° C. In other embodiments, the TGA thermogram of the Crystalline Form A exhibits a weight loss of about 10% in the temperature range of 25° C. to 250° C. In other embodiments, the TGA thermogram of the Crystalline Form A exhibits a weight loss of about 4.0% to about 7.0% in the temperature range of 60° C. to 220° C. In other embodiments, the TGA thermogram of the Crystalline Form A exhibits a weight loss of about 1.0% to about 3.0% in the temperature range of 230° C. to 270° C.

In one embodiment, Crystalline Form A can be synthesized from Crystalline Form 1 (Compound I-acetate).

### Crystalline Form B of Compound I-HCl (Bis-HCl Salt)

In one embodiment, the crystalline form of the Compound I-HCl is Crystalline Form B of (Crystalline Form B). In one embodiment, Crystalline Form B of Compound I-HCl exhibits an XRPD comprising one or more peaks at about 10 and 27 degrees two-theta with the margin of error of about ±0.5; about ±0.4; about ±0.3; about ±0.2; about ±0.1; about ±0.05; or less. In another embodiment, the XRPD of the Crystalline Form B further comprises one or more peaks at about 12.5 and 34.8 degrees two-theta with the margin of error of about ±0.5; about ±0.4; about ±0.3; about ±0.2; about ±0.1; about ±0.05; or less.

In one specific embodiment, the Crystalline Form B exhibits an XRPD that is substantially similar to FIG. 9 (bottom line).

In one embodiment, the Crystalline Form B exhibits a DSC thermogram comprising a broad endotherm between about 190° C. to about 275° C. In one embodiment, the Crystalline Form B exhibits a DSC thermogram comprising a broad exotherm between about 55° C. to about 150° C. In one specific embodiment, the Crystalline Form B exhibits a DSC thermogram that is substantially similar to FIG. 10 (bottom line of bottom set).

In one embodiment, the Crystalline Form B exhibits a TGA thermogram that is substantially similar to FIG. 10 (bottom line of top set). In other embodiments, the TGA thermogram of the Crystalline Form B exhibits a weight loss of about 0.0 to about 14% in the temperature range of 25° C. to 250° C. In other embodiments, the TGA thermogram of the Crystalline Form B exhibits a weight loss of about 12% in the temperature range of 25° C. to 250° C. In other embodiments, the TGA thermogram of the Crystalline Form B exhibits a weight loss of about 4.0% to about 8.0% in the temperature range of 55° C. to 220° C. In other embodiments, the TGA thermogram of the Crystalline Form B exhibits a weight loss of about 4.0% to about 8.0% in the temperature range of 225° C. to 290° C.

In one embodiment, Crystalline Form B can be synthesized from Crystalline Form 2 (Compound I free base tetrahydrate).

### Compound I-HCl Amorphous

In one embodiment, a solid form of Compound I can take an amorphous form of Compound I-HCl.

### Pharmaceutical Compositions

In one aspect, the present disclosure provides a pharmaceutical composition comprising a solid form of Compound I or a salt or a solvate thereof. In one embodiment, the composition comprises a solid form of Compound I or a pharmaceutically acceptable salt or solvate thereof. In one embodiment, a pharmaceutical composition comprises a crystalline form of Compound I or a salt or solvate thereof, as described herein. In one embodiment, a pharmaceutical composition comprises a therapeutically effective amount of a crystalline form of Compound I. In one embodiment, any one of pharmaceutical compositions described herein comprising a solid form of Compound I further comprises a pharmaceutically acceptable carrier or a pharmaceutically acceptable vehicle.

In one embodiment, a pharmaceutical composition comprises a crystalline form of Compound I solvate. In one embodiment, a pharmaceutical composition comprises a crystalline form of Compound I-acetate. In one embodiment, a pharmaceutical composition comprises a crystalline form of Compound I-hydrate. In one embodiment, a pharmaceutical composition comprises a crystalline form of Compound I-salt. In one embodiment, a pharmaceutical composition comprises a crystalline form of Compound I hydrochloric acid salt. In one embodiment, a pharmaceutical composition comprises at least on of Crystalline Form 1, Crystalline Form 2, Crystalline Form A, or Crystalline Form B as described herein.

In one embodiment, a pharmaceutical composition comprises Crystalline Form 2 of Compound I free base tetrahydrate.

In embodiment of the pharmaceutical compositions comprising Compound I free base tetrahydrate Form 2, the composition comprises a crystalline Compound I Form 1, Form 3, Form 4, Form 5, or Form 6 in an amount of less than about 5% by weight. In embodiment of the pharmaceutical compositions comprising Compound I free base tetrahydrate Form 2, the composition comprises a crystalline Compound I Form 1, Form 3, Form 4, Form 5, or Form 6 in an amount of less than about 1% by weight. In embodiment of the pharmaceutical compositions comprising Compound I free base tetrahydrate Form 2, the composition comprises a crystalline Compound I Form 1, Form 3, Form 4, Form 5, or Form 6 in an amount of less than about 0.5% by weight. In embodiment of the pharmaceutical compositions comprising Compound I free base tetrahydrate Form 2, the composition comprises less than about 0.5% by weight of each of crystalline Compound I Form 1, Form 3, Form 4, Form 5, or Form 6.

In embodiment of the pharmaceutical compositions comprising Compound I free base tetrahydrate Form 2, the composition comprises a crystalline Compound I Form 1, Form 3, Form 4, Form 5, or Form 6 in an amount of about 0.05% to about 50% by weight. In embodiment of the pharmaceutical compositions comprising Compound I free base tetrahydrate Form 2, the composition comprises a crystalline Compound I Form 1, Form 3, Form 4, Form 5, or Form 6 in an amount of about 0.05% to about 5% by weight, about 0.05% to about 10% by weight, about 0.05% to about 15% by weight, about 0.05% to about 20% by weight, about 0.05% to about 25% by weight, about 0.05% to about 30% by weight, about 0.05% to about 35% by weight, about 0.05% to about 40% by weight, about 0.05% to about 45% by weight, about 0.05% to about 50% by weight, about 0.05% to about 55% by weight, about 0.05% to about 60% by weight, about 0.05% to about 65% by weight, about 0.05% to about 70% by weight, about 0.05% to about 75% by weight, or about 0.05% to about 80% by weight.

In one embodiment, the present disclosure relates to a pharmaceutical composition comprising two or more crystalline form of Compound I, or a pharmaceutically acceptable salt, solvate, or hydrate thereof, selected from Crystalline Form A, Crystalline Form B, Crystalline Form 1, Crystalline Form 2, Crystalline Form 3, Crystalline Form 4, Crystalline Form 5, or Crystalline Form 6.

In one embodiment, a pharmaceutical composition as described herein can be useful for treating cancer. In another embodiment, a pharmaceutical composition as described herein can be useful for treating hematological malignancies.

In another embodiment, the present invention provides a pharmaceutical composition comprising a therapeutically effective amount of a crystalline form of Compound I, or a pharmaceutically acceptable salt, ester, and/or solvate thereof, as disclosed herein, as the active ingredient, combined with a pharmaceutically acceptable excipient or carrier. The excipients are added to the formulation for a variety of purposes.

In one embodiment, the present disclosure relates to solid formulation where the crystalline form of Compound I is maintained. In some embodiments, the present disclosure relates to formulation of various types as disclosed herein, prepared from a crystalline form of Compound I.

Diluents may be added to the formulations of the present invention. Diluents increase the bulk of a solid pharmaceutical composition, and may make a pharmaceutical dosage form containing the composition easier for the patient and care giver to handle. Diluents for solid compositions include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT®), potassium chloride, powdered cellulose, sodium chloride, sorbitol, and talc.

Solid pharmaceutical compositions that are compacted into a dosage form, such as a tablet, may include excipients whose functions include helping to bind the active ingredient and other excipients together after compression. Binders for solid pharmaceutical compositions include acacia, alginic acid, carbomer (e.g., carbopol), carboxymethylcellulose sodium, dextrin, ethyl cellulose, gelatin, guar gum, gum tragacanth, hydrogenated vegetable oil, hydroxyethyl cellulose, hydroxypropyl cellulose (e.g., KLUCEL), hydroxypropyl methyl cellulose (e.g., METHOCEL), liquid glucose, magnesium aluminum silicate, maltodextrin, methylcellulose, polymethacrylates, povidone (e.g., KOLLIDON, PLASDONE), pregelatinized starch, sodium alginate, and starch.

The dissolution rate of a compacted solid pharmaceutical composition in the patient's stomach may be increased by the addition of a disintegrant to the composition. Disintegrants include alginic acid, carboxymethylcellulose calcium, carboxymethylcellulose sodium (e.g., AC-DI-SOL and PRIMELLOSE), colloidal silicon dioxide, croscarmellose sodium, crospovidone (e.g., KOLLIDON and POLYPLASDONE), guar gum, magnesium aluminum silicate, methyl cellulose, microcrystalline cellulose, polacrilin potassium, powdered cellulose, pregelatinized starch, sodium alginate, sodium starch glycolate (e.g., EXPLOTAB), potato starch, and starch.

Glidants can be added to improve the flowability of a non-compacted solid composition and to improve the accuracy of dosing. Excipients that may function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

In some embodiment, the crystalline form of Compound I is maintained through the tableting process, including being under pressure from a punch and dye.

Flavoring agents and flavor enhancers make the dosage form more palatable to the patient. Common flavoring agents and flavor enhancers for pharmaceutical products that may be included in the composition of the present invention include maltol, vanillin, ethyl vanillin, menthol, citric acid, fumaric acid, ethyl maltol, and tartaric acid.

Solid and liquid compositions may also be dyed using any pharmaceutically acceptable colorant to improve their appearance and/or facilitate patient identification of the product and unit dosage level.

In liquid pharmaceutical compositions may be prepared using the crystalline forms of the present invention and any other solid excipients where the components are dissolved or suspended in a liquid carrier such as water, vegetable oil, alcohol, polyethylene glycol, propylene glycol, or glycerin.

Liquid pharmaceutical compositions may contain propylene glycol (PG) and/or macrogol (15)-hydroxystearate.

Liquid pharmaceutical compositions may contain emulsifying agents to disperse uniformly throughout the composition an active ingredient or other excipient that is not soluble in the liquid carrier. Emulsifying agents that may be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions may also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, and xanthan gum.

Sweetening agents such as aspartame, lactose, sorbitol, saccharin, sodium saccharin, sucrose, aspartame, fructose, mannitol, and invert sugar may be added to improve the taste.

Preservatives and chelating agents such as alcohol, sodium benzoate, butylated hydroxyl toluene, butylated hydroxyanisole, and ethylenediamine tetraacetic acid may be added at levels safe for ingestion to improve storage stability.

A liquid composition may also contain a buffer such as gluconic acid, lactic acid, citric acid or acetic acid, sodium gluconate, sodium lactate, sodium citrate, or sodium acetate. Selection of excipients and the amounts used may be readily determined by the formulation scientist based upon experience and consideration of standard procedures and reference works in the field.

The solid compositions of the present invention include powders, granules, aggregates and compacted compositions. The dosages include dosages suitable for oral, buccal, rectal, parenteral (including subcutaneous, intramuscular, and intravenous), inhalant and ophthalmic administration. Although the most suitable administration in any given case will depend on the nature and severity of the condition being treated, the most preferred route of the present invention is oral. The dosages may be conveniently presented in unit dosage form and prepared by any of the methods well-known in the pharmaceutical arts.

Dosage forms include solid dosage forms like tablets, powders, capsules, suppositories, sachets, troches and lozenges, as well as liquid syrups, suspensions, aerosols and elixirs.

The dosage form of the present invention may be a capsule containing the composition, preferably a powdered or granule solid composition of the invention, within either a hard or soft shell. The shell may be made from gelatin and optionally contain a plasticizer such as glycerin and sorbitol, and an opacifying agent or colorant.

A composition for tableting or capsule filling may be prepared by wet granulation. In wet granulation, some or all of the active ingredients and excipients in powder form are blended and then further mixed in the presence of a liquid, typically water that causes the powders to clump into granules. The granules are screened and/or milled, dried and then screened and/or milled to the desired particle size. The granules may be tableted, or other excipients may be added prior to tableting, such as a glidant and/or a lubricant.

A tableting composition may be prepared conventionally by dry blending. For example, the blended composition of the actives and excipients may be compacted into a slug or a sheet and then comminuted into compacted granules. The compacted granules may subsequently be compressed into a tablet.

As an alternative to dry granulation, a blended composition may be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate and colloidal silica. The proper use of these and other excipients in direct compression tableting is known to those in the art with experience and skill in particular formulation challenges of direct compression tableting.

A capsule filling of the present invention may comprise any of the aforementioned blends and granules that were described with reference to tableting; however, they are not subjected to a final tableting step.

In one embodiment, the crystalline form of Compound I, or a pharmaceutically acceptable salt and/or solvate thereof, is reconstituted prior to administration in pharmaceutically acceptable carrier or solvent. In one embodiment, the reconstituted solution formulation comprising Compound I, or a pharmaceutically acceptable salt and/or solvate thereof, is administered by an IV.

The active ingredient and excipients may be formulated into compositions and dosage forms according to methods known in the art.

In one embodiment, a dosage form may be provided as a kit comprising crystalline form of Compound I and pharmaceutically acceptable excipients and carriers as separate components. In some embodiments, the dosage form kit allow physicians and patients to formulate an oral solution or injection solution prior to use by dissolving, suspending, or mixing the crystalline form of Compound I with pharmaceutically acceptable excipients and carriers. In one embodiment, a dosage form kit which provides crystalline form of Compound I has improved stability of Compound I compared to pre-formulated liquid formulations of Compound I.

In one embodiment, any compositions and dosage forms disclosed herein can be prepared with any one of crystalline or non-crystalline forms of Compound I as disclosed herein.

### Pharmaceutical Compositions for Intravenous Formulation

In one embodiment of the present disclosure, an IV composition comprising a solid form of Compound I or a pharmaceutically acceptable salt or solvate thereof is provided. In one embodiment, a pharmaceutical IV composition comprises a crystalline form of Compound I or a pharmaceutically acceptable salt or solvate thereof. In one embodiment, a pharmaceutical IV composition comprises a crystalline form of Compound I solvate. In one embodiment, a pharmaceutical IV composition comprises a crystalline form of Compound I salt.

In one embodiment, a pharmaceutical IV composition comprises a crystalline form of Compound I-acetate. In one embodiment, a pharmaceutical IV composition comprises a crystalline form of Compound I-acetate Form 1.

In one embodiment, a pharmaceutical IV composition comprises a crystalline form of Compound I-hydrate. In one embodiment, a pharmaceutical IV composition comprises a crystalline form of Compound I free base hydrate. In one embodiment, a pharmaceutical IV composition comprises a crystalline form of Compound I tetrahydrate. In one embodiment, a pharmaceutical IV composition comprises a crystalline form of Compound I free base tetrahydrate. In one embodiment, a pharmaceutical IV composition comprises a crystalline form of Compound I free base tetrahydrate Form 2.

In one embodiment, a pharmaceutical IV composition comprises a crystalline form of Compound I-HCl. In one embodiment, a pharmaceutical IV composition comprises a crystalline form of Compound I bis-HCl salt. In one embodiment, a pharmaceutical IV composition comprises a crystalline form of Compound I-HCl Form A. In one embodiment, a pharmaceutical IV composition comprises a crystalline form of Compound I-HCl Form B.

In one embodiment of the present disclosure, an IV formulation comprising any one of the pharmaceutical compositions comprising Compound I or a pharmaceutically acceptable salt or solvate thereof as disclosed herein and an IV fluid is provided. In one embodiment, IV fluid is, but not limited to, sterile water, dextrose in water, glucose in water, invert sugar in water, saline solution in water (NaCl), sodium bicarbonate solution in water, sodium lactate solution in water, lactated Ringer's solution, or combinations thereof. In one embodiment, IV fluid is dextrose solutions, saline, half saline solution, neut, lactated Ringer's solution, and combinations thereof. In some embodiment, the IV fluid comprises 5% dextrose in water (D5W) or 10% dextrose in water (D10W). In another embodiment, the IV fluid comprises neut. In another embodiment, the IV fluid comprises D5W with neut or D10W with neut.

In one embodiment of the present disclosure, IV formulation comprising a crystalline form of Compound I or a pharmaceutically acceptable salt or solvate thereof passes through an in-line filter during infusion. In one embodiment, the IV formulation comprising a crystalline form of Compound I passes through an in-line filter greater than or equal to about 3 μm, 4 μm, 6 μm, 7 μm, 8 μm, 9 μm, or 10 In one embodiment, the IV formulation comprising a crystalline form of Compound I passes through an in-line filter greater than or equal to about 4 μm, 5 μm, 6 μm, 7 μm, 8 μm, 9 μm, or 10 μm for at least 120 minutes. In one embodiment, the IV formulation passes through an in-line filter of about 5 μm for at least 120 minutes.

In another embodiment, the IV formulation comprising a crystalline form of Compound I free base tetrahydrate Form 2 passes through an in-line filter of about 5 In one embodiment, the IV formulation comprising a crystalline form of Compound I free base tetrahydrate Form 2 passes through an in-line filter of about 5 μm for at least 120 minutes.

In one embodiment, the pharmaceutical composition comprising Compound I or a pharmaceutically acceptable salt or solvate thereof is stable for at least one month when stored at 25° C. in 60% relative humidity. In one embodiment, the pharmaceutical composition prepared from crystalline forms of Compound I or a pharmaceutically acceptable salt or solvate thereof is stable for at least one month when stored at 25° C. in 60% relative humidity.

In one embodiment, the chemical purity of Compound I or a pharmaceutically acceptable salt or solvate thereof in a pharmaceutical composition comprising Compound I or a pharmaceutically acceptable salt or solvate thereof remains greater than 95% when the pharmaceutical composition is stored at 25° C. in 60% relative humidity. In one embodiment, the chemical purity of Compound I or a pharmaceutically acceptable salt or solvate thereof in a pharmaceutical composition comprising Compound I or a pharmaceutically acceptable salt or solvate thereof remains greater than 96%, greater than 97%, greater than 98%, or greater than 99% when the pharmaceutical composition is stored at 25° C. in 60% relative humidity.

In one embodiment, the polymorphic purity of Compound I or a pharmaceutically acceptable salt or solvate thereof in a pharmaceutical composition comprising Compound I or a pharmaceutically acceptable salt or solvate thereof remains greater than 95% when the pharmaceutical composition is stored at 25° C. in 60% relative humidity.

In one embodiment, the pharmaceutical composition comprising a crystalline form of Compound I or a pharmaceutically acceptable salt or solvate thereof can be in a solution form. In one embodiment, the pharmaceutical composition comprising a crystalline form of Compound I or a pharmaceutically acceptable salt or solvate thereof is at a concentration of about 0.5 mg/mL to about 20 mg/mL of Compound I or a pharmaceutically acceptable salt or solvate thereof. In one embodiment, the pharmaceutical composition is at a concentration of about 0.5 mg/mL to about 10 mg/mL of Compound I or a pharmaceutically acceptable salt or solvate thereof. In some embodiments, the pharmaceutical composition is at a concentration of about 0.5 mg/mL, 1.0 mg/mL, 1.5 mg/mL, 2.0 mg/mL, 2.5 mg/mL, 3.0 mg/mL, 3.5 mg/mL, 4.0 mg/mL, 4.5 mg/mL, 5.0 mg/mL, 5.5 mg/mL, 6.0 mg/mL, 6.5 mg/mL, 7.0 mg/mL, 7.5 mg/mL, 8.0 mg/mL, 8.5 mg/mL, 9.0 mg/mL, 9.5 mg/mL, or 10.0 mg/mL of Compound I or a pharmaceutically acceptable salt or solvate thereof. In one embodiment, the pharmaceutical composition is at a concentration below 10 mg/mL of Compound I or a pharmaceutically acceptable salt or solvate thereof. In one embodiment, the pharmaceutical composition is at a concentration below 8 mg/mL of Compound I or a pharmaceutically acceptable salt or solvate thereof. In one embodiment, the pharmaceutical composition is at a concentration is at a range of about 3 mg/mL to about 5 mg/mL of Compound I or a pharmaceutically acceptable salt or solvate thereof.

In one embodiment, the pharmaceutical composition comprising a crystalline form of Compound I or a pharmaceutically acceptable salt or solvate thereof further comprises at least one pharmaceutically acceptable diluent selected from, but not limited to, water, saline, alkyleneglycols (e.g., propylene glycol), polyalkylene glycols (e.g., polyethylene glycol) oils, alcohols, slightly acidic buffers between pH 4 and pH 6 (e.g., acetate, citrate, ascorbate at between about 5 mM to about 50 mM), or the like. In one embodiment, the pharmaceutical composition comprising a crystalline form of Compound I or a pharmaceutically acceptable salt or solvate thereof further comprises propylene glycol. In another embodiment, the pharmaceutical composition comprising a crystalline form of Compound I or a pharmaceutically acceptable salt or solvate thereof further comprises water.

In one embodiment, the pharmaceutical composition comprising a crystalline form of Compound I or a pharmaceutically acceptable salt or solvate thereof further comprises propylene glycol in about 10% to about 90% by volume of the composition. In one embodiment, the pharmaceutical composition comprises propylene glycol in about 10%, 20%, 30%, 40%, 50%, 60%, 70%, 80%, or 90% by volume of the composition. In another embodiment, the pharmaceutical composition comprises propylene glycol in about 50%, 55%, 60%, 65%, 70%, 75%, 80% or 85% by volume of the composition. In one embodiment, the pharmaceutical composition comprises propylene glycol in about 70% by volume of the composition.

In one embodiment, the pharmaceutical composition comprising a crystalline form of Compound I or a pharmaceutically acceptable salt or solvate thereof further comprises water in less than about 50%, less than about 40%, less than about 30%, less than about 20%, or less than about 10% by volume of the composition. In one embodiment, the pharmaceutical composition comprises water in less than about 15%, 14%, 13%, 12%, 11%, 10%, 9%, 8%, 7%, 6%, 5%, or 4% by volume of the composition. In one embodiment, the pharmaceutical composition comprises water in about 4% to about 10% by volume of the composition.

In one embodiment, the pharmaceutical composition comprising a crystalline form of Compound I further comprises at least one pharmaceutically acceptable excipient selected from, but not limited to, macrogol (15)-hydroxystearate (e.g., Solutol® HS 15), egg lecithin, Polyoxy capryllic glyceride, polyoxy 10 oleyl ether, polyoxyethylene sorbitan fatty acid esters, ethanol, polyethylene glycol, or the like. In one embodiment, the pharmaceutical composition comprising a crystalline form of Compound I further comprises Solutol® HS 15.

In one embodiment, the pharmaceutical composition comprising a crystalline form of Compound I or a pharmaceutically acceptable salt or solvate thereof further comprises Solutol® HS 15 in about 5% to about 50% by volume of the composition. In one embodiment, the pharmaceutical composition comprises Solutol® HS 15 in about 5%, 10%, 20%, 30%, 40%, or 50% by volume of the composition. In one embodiment, the pharmaceutical composition comprises Solutol® HS 15 in about 10%, 11%, 12%, 13%, 14%, 15%, 16%, 17%, 18%, 19%, 20%, 21%, 22%, 23%, 24%, 25%, 26%, 27%, 28%, 29%, or 30% by volume of the composition. In one embodiment, the pharmaceutical composition comprises Solutol® HS 15 in about 20%, 21%, 22%, 23%, 24%, or 25% by volume of the composition.

In one embodiment, the pharmaceutical composition comprising a crystalline form of Compound I or a pharmaceutically acceptable salt or solvate thereof, the composition is substantially free of polyethylene glycol.

In one embodiment, the pharmaceutical composition comprising a crystalline form of Compound I or a pharmaceutically acceptable salt or solvate thereof comprises propylene glycol in about 60% to about 80%; Solutol® HS 15 in about 15% to about 30%; and water in about 3% to about 12%. In another embodiment, the pharmaceutical composition comprising a crystalline form of Compound I or a pharmaceutically acceptable salt or solvate thereof comprise propylene glycol in about 70%, Solutol® HS 15 in about 23%, and water in about 7%.

In one embodiment, in any one of the pharmaceutical compositions described herein, the crystalline form of Compound I or a pharmaceutically acceptable salt or solvate thereof is Compound I free base tetrahydrate Form 2.

In one embodiment, the pharmaceutical composition comprising a crystalline form of Compound I or a pharmaceutically acceptable salt or solvate thereof in propylene glycol, Solutol® HS 15 and water is a solution free of particles. In another embodiment, the pharmaceutical composition comprising a crystalline form of Compound I or a pharmaceutically acceptable salt or solvate thereof in propylene glycol, Solutol® HS 15 and water contains less than about 10%, about 9%, about 8%, about 7%, about 6%, about 5%, about 4%, about 3%, about 2%, about 1% total impurities. In one embodiment, the composition comprises less than or equal to 3% total impurities.

In one embodiment, the pharmaceutical composition comprising a crystalline form of Compound I in propylene glycol, Solutol® HS 15 and water, the average number of particles present does not exceed 6000 per container equal to or greater than 10 μm and does not exceed 600 per container equal to or greater than 25 μm.

In one embodiment, any pharmaceutical compositions disclosed herein can be prepared with any one of crystalline or non-crystalline forms of Compound I as disclosed herein.

### Methods of Use

In one aspect, the present disclosure provides methods of treating cancer in a subject in need thereof comprising administering to the subject a therapeutically effective amount of any one of the solid form of Compound I or a pharmaceutically acceptable salt or solvate thereof as described herein. In one embodiment, cancer is hematological malignancies. In one embodiment, hematological malignancies include leukemia and lymphoma. In further embodiments, the solid form of Compound I or a pharmaceutically acceptable salt or solvate thereof comprises a crystalline form of Compound I or a pharmaceutically acceptable salt or solvate thereof. In further embodiment, a crystalline form of Compound I includes solvates, hydrates, and salt including Compound I-acetate Form 1, Compound I free base tetrahydrate Form 2, Compound I-HCl Form A, and Compound I-HCl Form B, or mixtures thereof.

In another aspect of the present disclosure, any one of the solid forms of Compound I or a pharmaceutically acceptable salt or solvate thereof as described herein can be used to treat, stabilize or prevent cancer in a subject. In this context, the compounds may exert either a cytotoxic or cytostatic effect resulting in a reduction in the size of a tumour, the slowing or prevention of an increase in the size of a tumour, an increase in the disease-free survival time between the disappearance or removal of a tumour and its reappearance, prevention of an initial or subsequent occurrence of a tumour (e.g. metastasis), an increase in the time to progression, reduction of one or more adverse symptom associated with a tumour, or an increase in the overall survival time of a subject having cancer.

Exemplary tumours include, but are not limited to, haematologic neoplasms, including leukaemias, myelomas and lymphomas; carcinomas, including adenocarcinomas and squamous cell carcinomas; melanomas and sarcomas. Carcinomas and sarcomas are also frequently referred to as “solid tumours.” Examples of commonly occurring solid tumours include, but are not limited to, cancer of the brain, breast, cervix, colon, head and neck, kidney, lung, ovary, pancreas, prostate, stomach and uterus, non-small cell lung cancer and colorectal cancer. Various forms of lymphoma also may result in the formation of a solid tumour and, therefore, are also often considered to be solid tumours.

The cancers which can be treated in accordance with one embodiment of the present invention thus include, but are not limited to, leukaemias; adenocarcinomas and carcinomas, including squamous cell carcinomas. Carcinomas are also frequently referred to as “solid tumours,” as described above, and examples of commonly occurring solid tumours that can be treated in accordance with the present invention include, but are not limited to, anal cancer, bladder cancer, colon cancer, colorectal cancer, duodenal cancer, gastric (stomach) cancer, lung (non-small cell) cancer, oesophageal cancer, prostate cancer, rectal cancer and small intestine cancer. Accordingly, one embodiment of the present invention provides for the use of a compound of Formula I in the treatment of a cancer selected from the group of leukemia, bladder cancer, lung (non-small cell) cancer, prostate cancer and a cancer of the GI tract, wherein cancers of the GI tract include, but are not limited to, anal cancer, colon cancer, colorectal cancer, duodenal cancer, gastric (stomach) cancer, oesophageal cancer, rectal cancer and small intestine cancer.

One embodiment of the present disclosure provides for the use of any one of the solid form of Compound I or a pharmaceutically acceptable salt or solvate thereof as described herein in the treatment of one or more of prostate cancer, non-small cell lung cancer, colon cancer, renal cancer, pancreatic cancer, leukemia, lymphoma and/or brain cancer/tumour.

The term “leukaemia” or “leukemia” refers broadly to progressive, malignant diseases of the blood-forming organs. Leukaemia is typically characterized by a distorted proliferation and development of leukocytes and their precursors in the blood and bone marrow but can also refer to malignant diseases of other blood cells such as erythroleukaemia, which affects immature red blood cells. Leukaemia is generally clinically classified on the basis of (1) the duration and character of the disease—acute or chronic; (2) the type of cell involved—myeloid (myelogenous), lymphoid (lymphogenous) or monocytic, and (3) the increase or non-increase in the number of abnormal cells in the blood—leukaemic or aleukaemic (subleukaemic). Leukaemia includes, for example, acute nonlymphocytic leukaemia, chronic lymphocytic leukaemia, acute granulocytic leukaemia, chronic granulocytic leukaemia, acute promyelocytic leukaemia, adult T-cell leukaemia, aleukaemic leukaemia, aleukocythemic leukaemia, basophylic leukaemia, blast cell leukaemia, bovine leukaemia, chronic myelocytic leukaemia, leukaemia cutis, embryonal leukaemia, eosinophilic leukaemia, Gross' leukaemia, hairy-cell leukaemia, hemoblastic leukaemia, hemocytoblastic leukaemia, histiocytic leukaemia, stem cell leukaemia, acute monocytic leukaemia, leukopenic leukaemia, lymphatic leukaemia, lymphoblastic leukaemia, lymphocytic leukaemia, lymphogenous leukaemia, lymphoid leukaemia, lymphosarcoma cell leukaemia, mast cell leukaemia, megakaryocytic leukaemia, micromyeloblastic leukaemia, monocytic leukaemia, myeloblastic leukaemia, myelocytic leukaemia, myeloid granulocytic leukaemia, myelomonocytic leukaemia, Naegeli leukaemia, plasma cell leukaemia, plasmacytic leukaemia, promyelocytic leukaemia, Rieder cell leukaemia, Schilling's leukaemia, stem cell leukaemia, subleukaemic leukaemia, and undifferentiated cell leukaemia.

In one embodiment, any one of the solid forms of Compound I or a pharmaceutically acceptable salt or solvate thereof as described herein can be useful in treating acute myeloid leukemia (AML). In one embodiment, AML, is relapsed or refractory AML.

The term “carcinoma” refers to a malignant new growth made up of epithelial cells tending to infiltrate the surrounding tissues and give rise to metastases. The term “carcinoma” also encompasses adenocarcinomas. Adenocarcinomas are carcinomas that originate in cells that make organs which have glandular (secretory) properties or that originate in cells that line hollow viscera, such as the gastrointestinal tract or bronchial epithelia, and include adenocarcinomas of the lung and prostate.

In accordance with the present disclosure, any one of the solid forms of Compound I or a pharmaceutically acceptable salt or solvate thereof as described herein can be used to treat various stages and grades of cancer cell, tumor and/or cancer development and progression. The present disclosure, therefore, contemplates the use of the combinations in the treatment of early stage cancers including early neoplasias that may be small, slow growing, localized and/or nonaggressive, for example, with the intent of curing the disease or causing regression of the cancer, as well as in the treatment of intermediate stage and in the treatment of late stage cancers including advanced and/or metastatic and/or aggressive neoplasias, for example, to slow the progression of the disease, to reduce metastasis or to increase the survival of the patient. Similarly, the combinations may be used in the treatment of low grade cancers, intermediate grade cancers and or high grade cancers.

The present disclosure also contemplates that any one of the solid form of Compound I or a pharmaceutically acceptable salt or solvate thereof as described herein can be used in the treatment of indolent cancers, recurrent cancers including locally recurrent, distantly recurrent and/or refractory cancers (i.e. cancers that have not responded to treatment), metastatic cancers, locally advanced cancers and aggressive cancers. Thus, an “advanced” cancer includes locally advanced cancer and metastatic cancer and refers to overt disease in a patient, wherein such overt disease is not amenable to cure by local modalities of treatment, such as surgery or radiotherapy. The term “metastatic cancer” refers to cancer that has spread from one part of the body to another. Advanced cancers may also be unresectable, that is, they have spread to surrounding tissue and cannot be surgically removed.

One skilled in the art will appreciate that many of these categories may overlap, for example, aggressive cancers are typically also metastatic. “Aggressive cancer,” as used herein, refers to a rapidly growing cancer. One skilled in the art will appreciate that for some cancers, such as breast cancer or prostate cancer the term “aggressive cancer” will refer to an advanced cancer that has relapsed within approximately the earlier two-thirds of the spectrum of relapse times for a given cancer, whereas for other types of cancer, such as small cell lung carcinoma (SCLC) nearly all cases present rapidly growing cancers which are considered to be aggressive. The term can thus cover a subsection of a certain cancer type or it may encompass all of other cancer types.

The compounds may also be used to treat drug resistant cancers, including multidrug resistant tumors. As is known in the art, the resistance of cancer cells to chemotherapy is one of the central problems in the management of cancer.

Certain cancers, such as prostate, can be treated by hormone therapy, i.e. with hormones or anti-hormone drugs that slow or stop the growth of certain cancers by blocking the body's natural hormones. Such cancers may develop resistance, or be intrinsically resistant, to hormone therapy. The present invention further contemplates the use of the compounds in the treatment of such “hormone-resistant” or “hormone-refractory” cancers.

The compounds and compositions of the present disclosure may be used as part of a neo-adjuvant therapy (to primary therapy), or as part of an adjuvant therapy regimen. The present invention contemplates the use of the compounds of the present invention at various stages in tumor development and progression, including in the treatment of advanced and/or aggressive neoplasias (i.e. overt disease in a subject that is not amenable to cure by local modalities of treatment, such as surgery or radiotherapy), metastatic disease, locally advanced disease and/or refractory tumors (i.e. a cancer or tumor that has not responded to treatment).

“Primary therapy” refers to a first line of treatment upon the initial diagnosis of cancer in a subject. Exemplary primary therapies may involve surgery, a wide range of chemotherapies and radiotherapy. “Adjuvant therapy” refers to a therapy that follows a primary therapy and that is administered to subjects at risk of relapsing. Adjuvant systemic therapy is usually begun soon after primary therapy to delay recurrence, prolong survival or cure a subject.

It is contemplated that the compounds and the compositions of the disclosure can be used alone or in combination with one or more other chemotherapeutic agents as part of a primary therapy or an adjuvant therapy. Combinations of the compounds of the present invention and standard chemotherapeutics may act to improve the efficacy of the chemotherapeutic and, therefore, can be used to improve standard cancer therapies. This application can be important in the treatment of drug-resistant cancers which are not responsive to standard treatment. Drug-resistant cancers can arise, for example, from heterogeneity of tumor cell populations, alterations in response to chemotherapy and increased malignant potential. Such changes are often more pronounced at advanced stages of disease.

The present disclosure also contemplates the use of any one of the solid forms of Compound I or a pharmaceutically acceptable salt or solvate thereof as described herein as “sensitizing agents,” which selectively inhibit the growth of cancer cells. In this case, the compound alone does not have a cytotoxic effect on the cancer cell, but provides a means of weakening the cancer cells, and better facilitate the benefit obtained from the application of conventional anti-cancer therapeutics, or to otherwise potentiate said therapeutics.

Thus, the present disclosure contemplates the administration to a subject of a therapeutically effective amount of one or more of the solid forms of Compound I as described herein together with one or more anti-cancer therapeutics. The compound(s) can be administered before, during or after treatment with the anti-cancer therapeutic. An “anti-cancer therapeutic” is a compound, composition or treatment that prevents or delays the growth and/or metastasis of cancer cells. Such anti-cancer therapeutics include, but are not limited to, chemotherapeutic drug treatment, radiation, gene therapy, hormonal manipulation, immunotherapy and antisense oligonucleotide therapy. Examples of useful chemotherapeutic drugs include, but are not limited to, hydroxyurea, busulphan, cisplatin, carboplatin, chlorambucil, melphalan, cyclophosphamide, Ifosphamide, danorubicin, doxorubicin, epirubicin, mitoxantrone, vincristine, vinblastine, Navelbine® (vinorelbine), etoposide, teniposide, paclitaxel, docetaxel, gemcitabine, cytosine, arabinoside, bleomycin, neocarcinostatin, suramin, taxol, mitomycin C and the like. The compounds of the invention are also suitable for use with standard combination therapies employing two or more chemotherapeutic agents. It is to be understood that anti-cancer therapeutics for use in the present invention also include novel compounds or treatments developed in the future.

The dosage to be administered is not subject to defined limits, but it will usually be an effective amount. It will usually be the equivalent, on a molar basis of the pharmacologically active free form produced from a dosage formulation upon the metabolic release of the active free drug to achieve its desired pharmacological and physiological effects. The compositions may be formulated in a unit dosage form. The term “unit dosage form” refers to physically discrete units suitable as unitary dosages for human subjects and other mammals, each unit containing a predetermined quantity of active material calculated to produce the desired therapeutic effect, in association with a suitable pharmaceutical excipient. Examples of ranges for the compound(s) in each dosage unit are from about 0.05 mg to about 2000 mg.

A dosage form of the present invention may be administered, hourly, daily, weekly, or monthly. The dosage form of the present invention may be administered twice a day or once a day. The dosage form of the present invention may be administered with food or without food.

In one embodiment, compounds of the present invention or formulation prepared by compounds of the present invention, is administered once a week, once every two weeks, once every three weeks, once every four weeks, or once a month. In some embodiments, compounds of the present invention or formulation prepared by compounds of the present invention, is administered in a four-week treatment cycle comprising one administration weekly (QW×4). In some embodiments, compounds of the present invention or formulation prepared by compounds of the present invention, is administered in a four-week treatment cycle comprising one administration weekly for two weeks followed by two weeks of rest period (no treatment) (QW×2). In some embodiments, the administration is on a four-week treatment cycle comprising one administration weekly for three weeks followed by one week of rest period (no treatment). In some embodiments, compounds of the present invention or formulation prepared by compounds of the present invention, is administered in a three-week treatment cycle comprising one administration weekly for two weeks followed by one week of rest period. In another embodiment, compounds of the present invention or formulation prepared by compounds of the present invention, is administered once every three weeks. In other embodiments, compounds of the present invention or formulation prepared by compounds of the present invention, is administered once every three weeks by IV infusion.

In one embodiment, one IV infusion can comprise from about 25 mg of Compound I, or a pharmaceutically acceptable salt and/or solvate thereof to about 1000 mg of Compound I, or a pharmaceutically acceptable salt and/or solvate thereof.

In some embodiment, the treatment regimen with Compound I, or a pharmaceutically acceptable salt and/or solvate thereof, as disclosed herein, can last from 1 cycle to 20 cycles or greater period of time. An appropriate length of the treatment can be determined by a physician.

Dosages of the compounds of the present invention will typically fall within the range of about 0.01 to about 100 mg/kg of body weight, in single or divided dose. However, it will be understood that the actual amount of the compound(s) to be administered will be determined by a physician, in the light of the relevant circumstances, including the condition to be treated, the chosen route of administration, the actual compound administered, the age, weight, and response of the individual patient, and the severity of the patient's symptoms. The above dosage range is given by way of example only and is not intended to limit the scope of the invention in any way. In some instances dosage levels below the lower limit of the aforesaid range may be more than adequate, while in other cases still larger doses may be employed without causing harmful side effects, for example, by first dividing the larger dose into several smaller doses for administration throughout the day.

In one embodiment, any one of crystalline or non-crystalline forms of Compound I as disclosed herein can be administered in any one of the methods disclosed herein.

### EXAMPLES

The present invention is further illustrated by reference to the following Examples. However, it is noted that these Examples, like the embodiments described above, are illustrative and are not to be construed as restricting the scope of the invention in any way.

### Example 1: Polymorph Transition Study of Compound I-Acetate Form 1

A slurry of Compound I-acetate was prepared using various water-methanol solutions as indicated in Table 1. The Slurry was maintained at 20° C. or at 50° C. for one week then the resulting crystals were analyzed by XRPD to determine its polymorphic form.

TABLE 1
Transition of Compound I-Acetate Crystalline Form 1

| | | | Resulting |
|---|---|---|---|
| | | | Polymorph |
| | | | by XRPD |
| Solvent | Experiment | Appearance | Analysis |
| Water-Methanol (1:99) | Slurry 20° C., 1 week | White solid | Form 2 |
| Water-Methanol (5:95) | Slurry 20° C., 1 week | White solid | Form 2 |
| Water-Methanol (10:90) | Slurry 20° C., 1 week | White solid | Form 2 |
| Water-Methanol (25:75) | Slurry 20° C., 1 week | Yellow solid | Form 2 |
| Water-Methanol (50:50) | Slurry 20° C., 1 week | Yellow solid | Form 2 |
| Water-Methanol (1:99) | Slurry 50° C., 1 week | White solid | Form 1 |
| Water-Methanol (5:95) | Slurry 50° C., 1 week | White solid | Form 1 |
| Water-Methanol (10:90) | Slurry 50° C., 1 week | White solid | Form 1 |
| Water-Methanol (25:75) | Slurry 50° C., 1 week | Yellow solid | Form 2 |
| Water-Methanol (50:50) | Slurry 50° C., 1 week | Yellow solid | Form 2 |

The slurry experiments shown in Table 1 demonstrated that at 20° C., only 1% water was necessary to transition Crystalline Form 1 into Crystalline Form 2 (Compound I free base tetrahydrate). At 50° C., however, at least 25% water is needed for Crystalline Form 1 to transition into Crystalline Form 2. Without bound to any theory, the data presented in Table 1 indicates that Crystalline Form 2 is more stable than Crystalline Form 1 in the presence of water.

### Example 2: Effect of Drying on Crystalline Compound I-Tetrahydrate Form 2

Crystalline Compound I-tetrahydrate Form 2 was dried under varying temperature, pressure, and time as indicted in Table 2. After the drying, the resulting crystals were analyzed by XRPD to determine its polymorphic form.

TABLE 2
Transition of Crystalline Compound I-Tetrahydrate
Form 2 on Drying

| | | | Drying | Drying | Drying |
|---|---|---|---|---|---|
| Temp. | Pressure | Drying time | time | time | time |
| (° C.) | (mbar) | 1 h | 4 h | 24 h | 3 days |
| 15 | 600 | Form 2 | Form 2 | Form 2 | Form 2 |
| | 400 | Form 2 | Form 2 | Form 2 | Form 2 |
| | 0 | Form 2 | Form 1 | Form 1 | Form 1 |
| 25 | 600 | Form 2 | Form 2 | Form 2 | Form 2 |
| | 400 | Form 2 | Form 2 | Form 2 | Form 2 |
| | 0 | Form 2 | Form 1 | Form 1 | NT |
| 35 | 600 | Form 2 | Form 2 | Form 2 | NT |
| | 400 | Form 2 | Form 2 | Form 2 | NT |
| | 0 | Form 1 and Form 2 | Form 1 | Form 1 | NT |

NT = not tested

The drying study represented in Table 2 demonstrated that Crystalline Form 2 is stable under various drying conditions. Crystalline Form 1 was only observed after exposure to full vacuum (0 mbar). Under full vacuum for four hours, complete conversion to Form 1 was observed at all temperature tested. An anhydrous form of Compound I was not observed under any of the above drying conditions.

### Example 3: Compound I Bis-HCl Salt Formation

Initially, crystalline Compound I bis-HCl salt Form A was observed when Compound I-acetate Form 1 was used as the starting material in the HCl salt formation step with gas HCl whereas crystalline Compound I bis-HCl salt Form B was observed when Compound I-tetrahydrate Form 2 was used as the starting material in the HCl salt formation step with HCl(aq).

Different conditions were used to convert Compound I free base to Compound I bis-HCl salt as shown in Table 3 using 2 equiv. of HCl(aq). A slurry of Compound I free base and HCl(aq) in varying solvent (500 μL) was prepared and stirred overnight at the temperature indicated in Table 3, entries 1-8. In a cooling experiments as shown in Table 3, entries 9-12, Compound I free base was added to 1000 μL of the solvent and heated to 60° C. or reflux and held at this temperature for 1 hour. Subsequently, the solution was gradually cooled to 5° C. at a rate of 5° C./hour. In all of the experiments shown in Table 3, the resulting solids were analyzed by XRPD to determine the polymorphic forms.

TABLE 3
Compound I bis-HCl Formation on 50 mg scale using 2 equiv. HCl (aq)

| | | | | Resulting |
|---|---|---|---|---|
| | | | | HCl salt |
| | | | | Analyzed |
| Entry | Experiment* | Solvent | Appearance | by XRPD |
| 1 | Slurry, Room | N-methylpyrolidone | Yellow solid | Form C |
| | Temp. (RT) | | | |
| 2 | Slurry, RT | 2-ethoxyethanol | Yellow solid | Form D |
| 3 | Slurry, RT | Tetrahydrofuran | Yellow solid | Form B |
| 4 | Slurry, RT | 2-Butanone | Yellow solid | Form B |
| 5 | Slurry, 50° C. | N-methylpyrolidone | Yellow solid | Form C |
| 6 | Slurry, 50° C. | 2-ethoxyethanol | Yellow solid | Form D |
| 7 | Slurry, 50° C. | Tetrahydrofuran | Yellow solid | Form E |
| 8 | Slurry, 50° C. | 2-Butanone | Yellow solid | Form E |
| 9 | Slow cooling; | N-methylpyrolidone | Orange solid | Form C |
| | RT to | | | |
| | 60° C. to 5° C. | | | |
| 10 | Slow cooling; | 2-ethoxyethanol | Yellow solid | Amorphous |
| | RT to | | | |
| | 60° C. to 5° C. | | | |
| 11 | Slow cooling; | Tetrahydrofuran | Orange solid | Form A |
| | RT to | | | |
| | reflux to 5° C. | | | |
| 12 | Slow cooling; | 2-Butanone | Yellow solid | Form A |
| | RT to | | | |
| | reflux to 5° C. | | | |

*500 μL solvent used for slurry experiments and 1000 μL solvent used for slow cooling experiments.

Table 3 indicates that Compound I bis-HCl polymorph formation is dependent on solvent and temperature of the salt formation step.

### Example 4: Reproducibility of Compound I Bis-HCl Salt Formation

The reproducibility of the production of HCl salt polymorphs were tested on 0.5 g and 1 g scale with 20 mL of solvent and 5 equivalents of HCl (aq) as shown in Table 4 starting with Compound I free base.

TABLE 4
Compound I bis-HCl Formation using 5 equiv. HCl (aq)

| | | | | Resulting |
|---|---|---|---|---|
| | | | | HCl salt |
| | | | | Analyzed |
| Scale | Experiment | Solvent | Appearance | by XRPD |
| 0.5 g | Slow cooling; RT to | 2-Butanone | Orange/Yellow | Form B |
| | 60° C. to 20° C. | solid | | |
| 0.5 g | Slow cooling; RT to | Tetrahydrofuran | Orange/Yellow | Form A |
| | 40° C. to 20° C. | | solid | |
| 0.5 g | Slow cooling; RT to | Acetone | Orange solid | Form A |
| | 40° C. to 20° C. | | | |
| 0.5 g | Slow cooling; RT to | 2-Butanone | Orange solid | |
| | reflux to 20° C. | | | |
| 1 g | Slow cooling; RT to | 2-Butanone | Yellow solid | Form B |
| | reflux to 20° C. | | | |
| 1 g | Slow cooling; RT to | Acetone | Orange solid | |
| | 40° C. to 20° C. | | | |
| 1 g | Slow cooling; RT to | 2-Butanone | Yellow solid | Form B |
| | reflux to 20° C. | | | |

Table 4 demonstrates that Compound I bis-HCl salt polymorph formation is also dependent on scale. Compound I bis-HCl salt Form A can be prepared by cooling recrystallization in THF and Compound I bis-HCl salt Form B can be prepared by cooling recrystallization in 2-butanone. However, based on Examples 3 and 4, robust large scale manufacture of Compound I bis-HCl salt may be challenging.

### Example 5: Formulation Development with Compound I Free Base and Compound I HCl Salts

Previous formulation with Compound I HCl salt (Form A), Solutol® HS 15 (macrogol 15 hydroxystearate), propylene glycol (PG), polyethylene glycol-400 (PEG-400), and water (WFI=water for injection) faced challenges for IV administration due to filter clogging events. The previous formulation was an orange colored liquid that required storage under freezing conditions for stability. The objective of this study was to re-formulate Compound I HCl salt or prepare formulation with Compound I free base which would provide improved stability and eliminate filter clogging issues.

Various pharmaceutical compositions were prepared in order to develop a formulation suitable for IV administration. Table 5 demonstrates the optimization procedure for developing the pharmaceutical composition. A desirable IV pharmaceutical composition will be a solution. Further, a desirable IV pharmaceutical composition, when diluted with IV fluids will remain in solution. In addition, a desirable IV pharmaceutical composition, when diluted with IV fluids will remain in solution and pass an in-line filter of about 5 μm.

Clinical infusion simulation study: formulated Compound I (equivalent to 4 mg/mL Compound I free base tetrahydrate) was mixed in-line with Lactated Ringers IV Solution through a controlled delivery system employing infusion pumps. The blended liquid was passed through a 5 μm in-line filter and the eluate was then passed to the end of the infusion line. Sample analysis was performed by collecting materials at 0, 15, 30, 60, 75, 90, 105, 115 and 120 minutes and analyzing immediately be visual assessment under a microscope.

As indicated by Table 5, experiment nos. 1, 4-10, 13, and 17, a crystalline Compound I-HCl (bis-HCl salt) Form A was difficult to solubilize in varying concentration of Solutol® HS 15, propylene glycol (PG), polyethylene glycol-400 (PEG-400), and water. Compound I-HCl Form A was found to have insufficient solubility when formulated with the tested excipients. On the contrary, Compound I free base (such as Form 1 and Form 2) were more soluble than Compound I-HCl under the same conditions (e.g., compare entries 1 and 2). This was surprising because, usually salts are more soluble under aqueous conditions (e.g., water) than its counterpart free base. Therefore, the greater solubility of Compound I free base when compared to Compound I HCl salt was unexpected to the inventors.

Formulation using Compound I free base was tested under varying concentration of Solutol® HS 15, propylene glycol (PG), polyethylene glycol-400 (PEG-400), and water (exp. nos. 11-12, 14-16, and 18). The inventor experimented with different combinations of excipients used from in the previous formulation. PEG-400 did not improve solubility of Compound I free base in the tested conditions (entries 2 and 3). It was determined that PEG-400 did not add value, thus, PEG-400 was omitted from the formulation and the volume was replaced with PG.

Inventors observed, while adding water to the formulation, that the Compound I free base appeared more soluble in the beginning (approximately 3-10 mL added) while Compound I free base appeared insoluble (cloudy formulation) upon complete addition of water (up to 60 mL). With this observation in hand, the inventors reduced the amount of water content to observe its effect on Compound I free base solubility (exp. nos. 15, 16, and 18).

Upon reduction of water content to 3 mL (7% by volume), inventors observed clear solution which passed the clinical infusion procedure test, where no filter clogging was observed, and the pump flow rates never diminished. It was surprising to the inventors that the reduction of water content increased solubility of Compound I free base, because normally, increase in water would expect to increase solubility of free bases. Therefore, obtaining a clear solution with the formulation as indicated in experiment no. 18 was unexpected. The formulation according to experiment no. 18 did not require sonication and the formulation was stable at room temperature. Furthermore, the formulation according to experiment no. 18 demonstrated stability over at least one month under accelerated stability conditions at 25° C./60% RH (relative humidity) as shown in Table 6.

During the formulation optimization, the inventor found that it was beneficial to begin formulation by dispersing Compound I-tetrahydrate into a hot melt of Solutol® HS 15.

Furthermore, inventors discovered that the solubility was sensitive to PG manufacturer. Inventors found that Dow Chemical's PG was suited for preparing pharmaceutical composition for IV administration whereas Fischer Scientific's PG was not. When Fischer Scientific's PG was used to prepare the pharmaceutical composition, a clear solution could be obtained; however, upon addition of IV fluid Compound I crashed out of solution.

TABLE 5
Formulation Development Studies

| Exp. | Compd I Form | Hot Solutol | PG | PEG-400 | Water (WFI) | | Clinical Infusion Procedure |
|---|---|---|---|---|---|---|---|
| No. | Conc. (mg/mL) | mL (%)¹ | mL (%)¹ | mL (%)¹ | mL (%)¹ | Other Parameters | Test/Observations |
| 1 | Form A | 10 (10%) | 10 (10%) | 20 (20%) | 60 (60%) | Heated to 60-65° C., | Test: Did not pass |
| | 1 mg/mL | | | | | mixed, and | Form A (HCl salt) at 1 mg/mL failed |
| | | | | | | sonicated. | overnight solubility test for both |
| | | | | | | Prepared filtered | filtered and unfiltered solutions |
| | | | | | | (0.2 μM) sample | |
| | | | | | | and unfiltered | |
| | | | | | | sample | |
| 2 | Free Base | 10 (10%) | 10 (10%) | 20 (20%) | 60 (60%) | Change Compound | Test: Did not pass |
| | Form 2 | | | | | I form from HCl | Free base was more soluble than |
| | 1 mg/mL | | | | | salt (Form A) to | HCl salt (see Exp. No. 1). Addition |
| | | | | | | free base | of HCl (aq) enhanced solubility of |
| | | | | | | | the free base. PEG-400 reduced |
| | | | | | | | solubility. Insufficient solubility. |
| 3 | Free Base | 10 (10%) | 10 (10%) | 20 (20%) | 60 (60%) | Added DMSO | Test: Did not pass |
| | Form 2 | | | | | | Addition of DMSO did not enhance |
| | 1 mg/mL | | | | | | solubility of the free base. PEG-400 |
| | | | | | | | reduced solubility. Insufficient |
| | | | | | | | solubility. |
| 4 | Form A | 10 (10%) | 20 (22%) | 0 | 60 (67%) | Removed PEG-400 | Test: Did not pass |
| | 1 mg/mL | | | | | and increased PG | Increased PG in the absence of PEG- |
| | | | | | | | 400 enhanced solubility, but the |
| | | | | | | | overall solubility was insufficient. |
| 5 | Form A | 10 (10%) | 10 (10%) | 20 (20%) | 60 (60%) | Added HCl | Test: Did not pass |
| | 4 mg/mL | | | | | | Addition of HCl (aq) did not |
| | | | | | | | enhance solubility of HCl salt (Form |
| | | | | | | | A) |
| 6 | Form A | 10 (10%) | 30 (22%) | 0 | 60 (66%) | Added HCl and | Test: Did not pass |
| | 4 mg/mL | | | | | removed PEG-400 | Addition of excess HCl (aq) did not |
| | | | | | | | enhance solubility of HCl salt (Form |
| | | | | | | | A) in the absence of PEG-400 and |
| | | | | | | | increased amount of PG. |
| 7 | Form A | 10 | 10 | 20 | 40 | Used 20% less | Test: Did not pass |
| | 4 mg/mL | | | | | water | Decreased amount of water did not |
| | | | | | | | enhance solubility of HCl salt. |
| 8 | Form A | 10 | 10 | 20 | 80 | Used 20% more | Test: Did not pass |
| | 4 mg/mL | | | | | water | Increased amount of water did not |
| | | | | | | | enhance solubility of HCl salt. |
| 9 | Form A | 10 (10%) | 30 (30%) | 0 | 60 (60%) | Removed PEG-400 | Test: Did not pass |
| | 4 mg/mL | | | | | and substituted with | Substitution of PG for PEG-400 did |
| | | | | | | PG | not enhance solubility of HCl salt. |
| 10 | Form A | 10 (10%) | 30 (30%) | 0 | 60 (60%) | Added 10% excess | Test: Did not pass |
| | 4 mg/mL | | | | | of HCl | Substitution of PG for PEG-400 did |
| | | | | | | | not enhance solubility of HCl salt |
| | | | | | | | even with addition of excess HCl |
| | | | | | | | (aq). |
| 11 | Free Base | 10 (10%) | 30 (30%) | 0 | 60 (60%) | Removed PEG-400 | Test: Did not pass |
| | Form 2 | | | | | and substituted with | Substitution of PG for PEG-400 did |
| | 4 mg/mL | | | | | PG with free base | not enhance solubility of free base. |
| 12 | Free Base | 10 (10%) | 30 (30%) | 0 | 60 (60%) | Added 10% excess | Test: Did not pass |
| | 4 mg/mL | | | | | of HCl | Substitution of PG for PEG-400 did |
| | | | | | | | not enhance solubility of free base |
| | | | | | | | even with addition of excess HCl |
| | | | | | | | (aq). |
| 13 | Form A | 10 (10%) | 30 (30%) | 0 | 60 (60%) | Concentration of | Test: Did not pass |
| | 2 mg/mL | | | | | Compound I | Substitution of PG for PEG-400 did |
| | | | | | | reduced | not enhance solubility of HCl salt |
| | | | | | | | even when concentration reduced to |
| | | | | | | | 2 mg/mL. |
| 14 | Free Base | 10 (10%) | 30 (30%) | 0 | 60 (60%) | Add acidified water | Test: Did not pass |
| | Form 2 | | | | Acidified | with 10% excess of | Substitution of PG for PEG-400 did |
| | 2 mg/mL | | | | WFI | HCl | not enhance solubility of free base |
| | | | | | | | even with addition of excess HCl |
| | | | | | | | (aq) at reduced concentration of 2 |
| | | | | | | | mg/mL. |
| 15 | Free Base | 10 (12.5%) | 30 | 0 | 40 (50%) | Use 10% less water | Test: Did not pass |
| | Form 2 | | (37.5%) | | | | Substitution of PG for PEG-400 and |
| | 2 mg/mL | | | | | | reduction of water did not enhance |
| | | | | | | | solubility of free base even at |
| | | | | | | | reduced concentration of 2 mg/mL. |
| 16 | Free Base | 10 (20%) | 30 (60%) | 0 | 10 (20%) | Use 40% less water | Test: Did not pass |
| | Form 2 | | | | Acidified | and use acidified | Substitution of PG for PEG-400 and |
| | 2 mg/mL | | | | WFI | water (2 eq of HCl) | reduction of water did not enhance |
| | | | | | | | solubility of free base even with |
| | | | | | | | excess HCl and at reduced |
| | | | | | | | concentration of 2 mg/mL. |
| 17 | Form A | 10 (23%) | 30 (70%) | 0 | 3 (7%) | Use 53% less water | Test: Did not pass |
| | 4 mg/mL | | | | Acidified | and use acidified | Substitution of PG for PEG-400 and |
| | | | | | WFI | water | significant reduction of water did not |
| | | | | | | | enhance solubility of HCl salt even |
| | | | | | | | excess HCl. |
| 18 | Free Base | 10 (23%) | 30 (70%) | 0 | 3 (7%) | Use 53% less water | Test: Pass |
| | Form 2 | | | | | and no acid | Generated clear yellow solution with |
| | 4 mg/mL | | | | | | no visible particles. Solution |
| | | | | | | | remained clear and passed the |
| | | | | | | | clinical infusion procedure testing. |

¹% by volume

TABLE 6
Stability Test Results at 25° C./60% RH (Compound I free base tetrahydrate formulation according
to experiment no. 18 in Table 5)

| | | Initial Sample | Sample 1 at | Sample 2 at |
|---|---|---|---|---|
| | Limit | (t = 0) | 1 month | 1 month |
| Appearance | Report Results | Yellow translucent solution | Yellow transparent solution | Yellow transparent solution |
| | | in a 20 mL clear glass vial | in a 20 mL clear glass vial | in a 20 mL clear glass vial |
| | | with grey stopper and green | with grey stopper and green | with grey stopper and green |
| | | flip off overseal containing | flip off overseal containing | flip off overseal containing |
| | | 20 mL | 20 mL | 20 mL |
| pH | Report Results | 7.3 − standard probe | 8.0 | 8.0 |
| | | 8.5 − micro probe | | |
| Assay (HPLC) | Report Results | 101.4 | 100.4 | 101.1 |
| | (% LC) | | | |
| Related Substances | | | | |
| (RS) | | | | |
| 1,10-phenanthroline-5,6-dione | Report Results | <0.10 | <0.10 | <0.10 |
| 1,10-phenanthroline | (% LC) | <0.10 | <0.10 | <0.10 |
| 5-fluoro-2-methylindole-3- | | <0.10 | <0.10 | <0.10 |
| carboxyaldehyde | | | | |
| 5-fluoro-2-methylindole | | 0.17 | 0.17 | 0.18 |
| Largest Unknown | | 1.12/0.25 | 1.09/0.18 | 1.09/0.14 |
| (RRT %) | | | | |
| Total RS | | 0.44 | 0.35 | 0.32 |
| Particulate Matter | Particles | 459 | NA | NA |
| | ≥10 μm/vial | | | |
| | Particles | 52 | NA | NA |
| | ≥25 μm/vial | | | |

### Example 6: IV Formulation IV Infusion Simulation

Materials and Equipment

IV infusion pump: Carefusion/Cardinal Health/Alaris, ALARIS 8015

IV Bag: BBraun Partial Additive Bag (PAB); Empty 150 mL PAB Bag (HETP free)

Syringe (20 or 60 mL): BD 302830 or BD 309653; BD Sterile Luer Lok Syringes

Low Sorbing Infusion Set: Carefusion, REF 2260-0500; low sorbing extension set for pump infusion (approx 23 mL priming volume).

‘Y’ Extension Set: Carefusion, REF MP2202-C; Y line for infusion with Luer Lok connections (approx 1.2 mL priming volume).

Extension Set with Filter: ICU Medical, Inc., B90003; extension set with filter (5 micron) (approx 2.6 mL priming volume)

Needle: BD 305175 or BD 305196; Sterile Precision Glide 20G or 18G Needle

Micropipette: Gilson (100 μL); micropipette sampling, 25 μL sample taken for microscopy analysis.

Microscope slides: Hemocytometer, Clay Adam, Cat. 1490. Model: 4011 Pre-cleaned glass microscope slides.

Microscope: Nikon ECLIPSE 50i, 125v, Nikon INTENSILIGHT C-HGFI DS Camera Control Unit DS-U2; Software: NIS-Elements BR3.2; 10×10 lens; Hemocytometer: Clay Adam, Cat. 1490. Model: 4011.

Lactated Ringer's Solution: B Braun, Lot #: J5D140; Container: Excel® plastic bag; Fill volume: 1000 mL; Storage: Room temperature

Pharmaceutical composition as described in Table 5, entry 18 (PG:Solutol® HS 15:USP Water for Injection in a 70:23:7 v/v/v solution) was tested to in a IV infusion simulation study. This study was conducted to demonstrate that the optimized IV formulation using Compound I-tetrahydrate (Form 2) does not clog the in-line filter during IV administration using “Y-line” infusion set.

For this simulated studies Compound I-tetrahydarte (4 mg/mL) was mixed in-line with Lactated Ringers IV Solution through a controlled delivery system employing infusion pumps. The blended liquid then passed through an in-line filter (5 μm) and the eluate then passed to the end of the infusion line. This was performed with the 220 mg/m² dosage level

Sample analysis was performed by collecting materials at 0, 15, 30, 60, 75, 90, 105, 115 and 120 minutes and analyzing immediately (within about 1 min) after being collected be visual assessment under a microscope. In this simulated infusion studies, no filter clogging was observed and the pump flow rates never diminished.

### Example 7: IV Formulation End User Compatibility Study

Materials and Equipment

A pharmaceutical composition comprising Compound I-tetrahydrate was diluted for use in a running IV line at a high and low strength concentration as follows:

For the high concentration, 150 mL of Compound I-tetrahydrate (4.0 mg/mL) was injected into a BBraun partial additive bag (PAB) for IV administration, attached to a Y-infusion set line. The other end of the Y infusion set line was connected to Lactated Ringer's Solution. Infusion is performed via the Y infusion set, such that the drug product is mixed with Lactated Ringer's Solution directly prior to administration. A total volume of 100 mL drug product at a rate of 50 mL/hr and 200 mL Lactated Ringer's Solution at a rate of 100 mL/hr were administered in a 2:1 ratio over a two hour period, to deliver 300 mL of eluent at a rate of 150 mL/hr.

In USP <788> monograph, two methods were provided to determine particles or particulate matter in an injectable drug, i.e. using an analytical instrument to count particle or Method I (e.g. electronic light obscuration or HIAC method) or counting the particles using an optical microscope (Method 2). Prior to conducting the End-User Compatibility Study, a study was performed to determine the feasibility of sample collection and assessment by USP <788> Method I (HIAC analysis). For this study, Compound I-tetrahydrate (4 mg/mL) was mixed with Lactated Ringers IV Solution in a 2:1 ratio of Lactated Ringers solution to Compound I-tetrahydrate and held in a glass or Nalgene plastic container. Samples were held for 1, 5, 10, 15, 20, 25 and 30 minutes and analyzed by USP <788> Method 1 using a HIAC electronic light obscuration particle counter. Results demonstrated that particulate would form at levels exceeding USP acceptance criteria of not more than (NMT) 3000 particles per container ≥10 μm and NMT 300 per container ≥25 μm at approximately 10 minutes. This short time duration precluded the use of USP <788> Method 1 (HIAC) due to the large sample volumes (˜25 mL) and length of time required to collect this volume of sample, drop wise, from the infusion set. Similarly, it does not allow for large sample volume collection and filtration as required by USP <788> Method 2.

The evaluation of particulate matter from collected IV samples was therefore performed by microscopic visual examination utilizing approximately 25 μL of IV co-infusion eluate, collected directly from the terminal end of the IV line, deposited on a microscope slide. The sample was deposited on a microscope slide (a hemocytometer) for Particulate Matter Testing, and the following criteria were applied: “The average number of particles present does not exceed 3000 per container equal to or greater than 10 μm and does not exceed 300 per container equal to or greater than 25 μm.” A microscopic photograph of each collected sample as well as blanks were recorded, and photographs taken to document results. The specific microscope settings used are shown below. A Positive Control sample containing a USP Particle Count Reference Standard consisting of spherical particles of known sizes between 10 μm and 15 μm (USP reference standard, Cat. No.: 1500502, lot #L0L142) was visualized under the microscope using a 10×10 lens.

No solid particles were seen anywhere inside the infusion set within the 120 min simulated infusion run, and no particles were detected by the microscope examination in any of the eluent sample collected. The flow was smooth and the pumps delivered the expected amount of eluent. No filter clogging or stoppage of the infusion pump was observed. These microscopic findings illustrate that the HIAC data are artefactual and do not represent the particle-free material that is actually delivered to the patient via the eluate exiting the IV infusion tubing.

This study demonstrated that the material may be infused for up to 2 hours without filter clogging when administered using a “Y-Line” infusion set.

### Example 8: In Vitro Antiproliferative Assay Against Acute Myeloid Leukemia Cell Assay

Compound I free base and Compound I HCl salt were both tested for their in vitro antiproliferative potency against acute myeloid leukemia (AML) cell lines, for their concentration-dependent abilities to induce in vitro changes in gene expression (KLF4, c-Myc, CDX2, p21, and GAPDH genes) and to induce cell cycle arrest and apoptosis in AML cell lines. In every assay performed, the free base and the HCL salt of Compound I behaved equivalently.

### Example 9: Rat Pharmacokinetics (PK) Study

A GLP, single-dose intravenous (IV) PK study is planned in Sprague-Dawley rats with the new formulation with Compound I-tetrahydrate. The study is planned to divide the rat population in 4 groups to administer Compound I-HCl salt in 10% Solutol HS-15, 20% PEG-400, and 10% PG in water diluted in D5W at two different doses (low dose and high dose) using a single IV infusion pump as well as Compound I-tetrahydrate (free base) in 23% Solutol HS-15 and 70% PG in water with co-administration of Lactated Ringer's solution using dual IV infusion pump at two different doses (low dose and high dose). For the groups receiving Compound I-tetrahydrate, the Compound I-tetrahydrate solution and Lactated Ringer's solution will be infused for 2 hours simultaneously using two separate infusion pumps and a Y connector to mimic the Y-infusion set.

### Example 10: Synthesis of Crystalline Compound I-Hydrate Form 2

To a 10-L, jacketed reactor was charged crude Compound I freebase (438 g), isopropyl alcohol (IPA, 2.97 L, 6.8 vol), and ammonium hydroxide (NH₄OH, 1.27 L, 2.9 vol). The resulting slurry was heated to 50° C. and stirred 4 h. The batch was then cooled to 20° C. over 4 h and stirred 14 h. The batch was then filtered through a polypropylene cloth and washed twice with 2:1 IPA/water (876 mL, 2 vol) followed by washing three times with DI water (6×1.31 L, 6×3 vol). The filtration was very slow and was changed to a sharkskin filter paper after the first water wash. The batch was then dried at 30-40° C. in a vacuum oven to give 198 g crude Compound I-hydrate (70.5% yield, 99.3 area % by HPLC). The batch was returned to the 10-L reactor with acetone (1.34 L, 3 vol) and DI water (571 mL, 1.3 vol), followed by heating to 50° C. The batch dissolved completely and was stirred for 4 h at 50° C. The batch was then cooled to 20° C. over 4 h and stirred 10 h prior to filtering through polypropylene cloth. The filter cake was washed twice with 2:1 acetone/DI water, followed by three washes with DI water. The batch was then dried at 30-40° C. in a vacuum oven to give 187 g Compound I-tetrahydrate Form 2 (94.9% recovery, 99.8 area % by HPLC; 16.7% w/w water by KF).

TGA thermogram of Compound I-hydrate Form 2 is shown in FIG. 12. The TGA thermogram shows overlapping large weight losses from 38° C. to 137° C. (9.9 wt %) and from 137° C. to 182° C. (6.5 wt %). The total loss (16.4 wt %) is equivalent to 4-5 moles of H₂O as the amount of organic solvents observed by NMR is insufficient to account for such an appreciable weight loss.

DSC thermogram of Compound I-hydrate Form 2 is shown in FIG. 13. DVS isotherm plot of Compound I-tetrahydrate Form 2 is shown in FIG. 13. The DSC thermogram displays two broad endotherms at 128° C. and 166° C. (peak max). These are concurrent with the weight losses seen by TGA and are likely due to desolvation, based on hotstage microscopy observations. There is also a sharp exotherm present at 217° C. (peak max), the nature of which is unknown.

Compound I-hydrate Form 2 was also analyzed by DVS (FIG. 14). Form 2 steadily gained 1.1 wt % from 5-95% RH. It underwent complete desorption with minor hysteresis. No form change was apparent, based on XRPD of the post-DVS solids.

Compound I-hydrate Form 2 was determined to be composed of a single crystalline phase based on XRPD analysis (FIG. 11). Form 2 has a monoclinic unit cell containing eight molecules of Compound I. The unit cell volume, calculated from the indexing solution, was consistent with a tetrahydrate.

### Example 11. Crystalline Compound I-Hydrate Form 3

Crystalline Compound I-Hydrate Form 3 was prepared by drying Crystalline Compound I-Hydrate Form 2 over P₂O₅ under vacuum for three hours.

XRPD spectrum was obtained for Compound I-hydrate Form 3 (FIG. 15B).

DSC thermogram of Compound I-hydrate Form 3 is shown in FIG. 16. TGA thermogram of Compound I-hydrate Form 3 is shown in FIG. 17. DVS isotherm plot of Compound I-tetrahydrate Form 3 is shown in FIG. 18.

### Example 12. Crystalline Compound I Form 4

Crystalline Form 4 was prepared by heating Crystalline Compound I-Hydrate Form 2 at 180° C. or 220° C. Crystalline Form 4 obtained by heating at 180° C. contained Crystalline Form 3. Crystalline Form 4 obtained by heating at 220° C. contained Crystalline Form 3 and Crystalline Form 6.

XRPD spectrum was obtained for Crystalline Form 4 (FIG. 19A, top spectrum and second from top spectrum). Based on XRPD, Form 4 is crystalline with disorder.

### Example 13. Crystalline Compound I Form 5

Crystalline Form 5 was prepared by heating a slurry of Compound I (free base) in butanol to 65° C., then slow cooling to slurry mixture to room temperature.

XRPD spectrum was obtained for Crystalline Form 5 (FIG. 20). XRPD demonstrated that Form 5 composed of a single crystalline phase with an orthorhombic unit cell containing four molecules of Compound I. The unit cell volume, calculated from the indexing solution, is consistent with one mole of butanol per molecule of Compound I. DSC and TGA thermograms of Crystalline Form 5 is shown in FIG. 21. The DSC thermogram shows a small endotherm at 153° C. (peak max) followed by a large endotherm at 179° C. (peak max; onset at 170.5° C.). The larger endotherm is likely due to desolvation as it occurs above the boiling point of 1-butanol. Negligible weight loss is observed in the TGA thermogram for Form 5 below 121° C. A 17 wt % loss is seen between 121° C. and 202° C. and is equivalent to approximately 1 mol of butanol.

### Example 14. Crystalline Compound I Form 6

Crystalline Form 6 was prepared by a slurry of Compound I (free base) in anhydrous acetone and stirring at room temperature for about 2.5 weeks.

XRPD spectrum was obtained for Crystalline Form 6 (FIG. 22). XRPD demonstrated that Form 6 composed of a single crystalline phase with an orthorhombic unit cell containing four molecules of Compound I. The unit cell volume, calculated from the indexing solution, is consistent with an anhydrous and unsolvated Compound I.

TGA thermogram of Crystalline Form 6 is shown in FIG. 23. TGA thermogram shows a 0.4 wt % loss from 38° C. to 182° C., which could be attributable to about 0.1 moles of H₂O or 0.03 moles of acetone.

### Example 15. Variable Relative Humidity (VRH)-XRPD Analysis of Crystalline Compound I Form 2

To investigate the behavior of Crystalline Form 2 at various humidities and evaluate conditions that favor generation of Form 2 (tetrahydrate) versus Form 3 (dihydrate), Form 2 was characterized by VRH-XRPD. The analysis was conducted by beginning at ambient RH (˜50%) and lowering to 0% RH for 4 hours. Actual RH during this time ranged from 0.7-0.3%. Humidity was then increased to 50% RH and 81% RH, each for 1 hour, and finally to 86% RH for 2 hours before cycling back to 80% RH, 50% RH, and ambient RH (˜40-37%), each also for 1 hour.

Based on the data, Form 3 was present in significant quantities after the first scan at ˜0% RH. Over time at 0% RH, the amount of Form 3 increases while the amount of Form 2 continually decreases. After increasing the RH to 50%, Form 2 is apparent again by)(RFD, but trace amounts of Form 3 likely remain through 86% RH.

To determine relative humidity boundaries for conversion between Form 2 and Form 3, solids of Form 2 were exposed to P₂O₅ conditions (˜0% RH), 11%, and 23% RH. The solids were held at the specified humidities for 11 days to see if the longer duration would result in the presence of Form 3. Based on the data, the samples from 11% and 23% RH are consistent with Form 2 and no form change was observed. This suggests that Form 2 is physically stable at ˜11% RH and above, when exposed for over a week. Under P₂O₅ conditions (˜0% RH), Form 2 converts to Form 3.

TABLE 6
VRH-XRPD Analysis

| Conditions | Results |
|---|---|
| Initial scan at ambient RH (46.3%) | Consistent with Form 2 |
| Held at ~0.3-0.7% RH, 4 hours | Form 2 + Form 3; amount of Form 3 |
| RH at start of first scan: ~0.7% | increases over time, amount |
| RH at end of final scan: ~0.3% | of Form 2 decreases over time |
| Held at ~50% RH, 1 hour | Form 2 + Form 3 |
| Held at ~82-79% RH, 1 hour | Form 2 + trace amount of Form 3 |
| Held at ~85-87% RH, 2 hours | Form 2, possibly contains trace |
| | amount of Form 3 |
| Held at ~80% RH, 1 hour | Form 2, possibly contains trace |
| | amount of Form 3 |
| Held at ~50% RH, 1 hour | Form 2, possibly contains trace |
| | amount of Form 3 |
| Held at ambient RH | Form 2 + trace amount of Form 3 |
| RH at end of first scan: ~40% | |
| RH at end of final scan: ~37% | |

### Example 16. Single Dose Intravenous Infusion in Rats

Compound I-hydrate Form 2 and Compound I-HCl salt were administered to Sprague-Dawley rats (6 rats per dose group) once by intravenous infusion via a tail vein over a 5-minute period at 0.5 mg/kg dose (calculated based on equivalent weight of Compound I free base). Compound I-hydrate Form 2 was formulated in 23% Solutol HS-15 and 70% PG and diluted with Lactated Ringer Solution to a concentration of 0.061 mg/mL. Compound I-HCl salt was formulated in 10% Solutol HS-15, 20% PEG_400, and 10% PG, and diluted with D5W to a concentration of 0.061 mg/mL. Dose volume was 10 mL/Kg and the infusion rate was 120 mL/kg/hr.

Following dosing, a series of 12 blood samples (approximately 0.2 mL each) were collected from the rats in the first 72 hours. Following collection, blood samples were allowed to stand at room temperature for approximately 30 to 60 minutes to clot and then centrifused (1200×g for 10 minutes at approximately 4° C.) and the resulting serum was recovered and stored frozen until analysis. Mean toxicokinetic parameters were calculated (Table 7).

TABLE 7
Mean toxicokinetic Parameters in Male Rats Serum at 0.5 mg/kg Dose

| | Comp I-hydrate | Comp |
|---|---|---|
| | Form 2 | I-HCl salt |
| T_{1/2} (hr) | 1.45 | 0.25 |
| (Terminal elimination half-life) | | |
| Tₘₐₓ (hr) | 0.00 | 0.00 |
| (time to maximum plasma concentration) | | |
| Cₘₐₓ (ng/mL) | 1750 | 590 |
| (maximum plasma concentration) | | |
| AUC_{0-Tlast} (hr*ng/mL) | 100 | 34 |
| (area under the plasma drug concentration- | | |
| time curve from the time of dosing | | |
| extrapolated to infinity) | | |
| AUC_{INF} (hr*ng/mL) | 130 | 35 |
| (area under the plasma drug concentration- | | |
| time curve from the time of dosing to the last | | |
| quantifiable concentration) | | |
| Cl (mL/hr/kg) | 3843 | 14192 |
| (total body clearance per kg body weight) | | |
| Vz (mL/kg) | 8037 | 5140 |
| (volume of distribution per kg body weight) | | |
| AUC % Extrap (%) | 23.1 | 3.70 |

Notably, Cₘₐₓ and AUCs were approximately 3 fold higher with Compound I-hydrate Form 2 (tetrahydrate) than with Compound I-HCl salt. Even though the volume of distribution was slightly higher (1.6 fold) for Compound I-hydrate Form 2, the rate of total body clearance per kg body weight was 3.7 fold higher for the Compound I-HCl salt than Compound I-hydrate Form 2.

### INCORPORATION BY REFERENCE

All references, articles, publications, patents, patent publications, and patent applications cited herein are incorporated by reference in their entireties for all purposes. However, mention of any reference, article, publication, patent, patent publication, and patent application cited herein is not, and should not be taken as acknowledgment or any form of suggestion that they constitute valid prior art or form part of the common general knowledge in any country in the world.

1. A crystalline form of Compound I free base tetrahydrate.

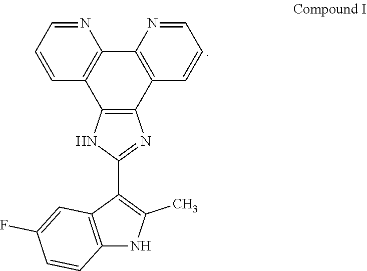 [SMILES: Cc1[nH]c2ccc(F)cc2c1-c1nc2c3cccnc3c3ncccc3c2[nH]1]

2. The crystalline form of claim 1, wherein the crystalline form is substantially pure.

3. The crystalline form of claim 1, wherein the crystalline form of Compound I free base tetrahydrate has a chemical purity of greater than about 95%.

4. (canceled)

5. The crystalline form of claim 1, wherein the crystalline form of Compound I free base tetrahydrate has a chemical purity of greater than about 99%.

6. The crystalline form of claim 1, which exhibits an X-ray powder diffraction (XRPD) pattern substantially similar to FIG. 11.

7. (canceled)

8. The crystalline form of claim 1, which exhibits an XRPD pattern comprising peaks at 10.0±0.2 and 25.0±0.2 degrees two-theta.

9. The crystalline form of claim 8, which exhibits an XRPD pattern comprising peaks at 26.3±0.2 and 28.2±0.2 degrees two-theta.

10. The crystalline form of claim 8, which exhibits an XRPD pattern comprising peaks at 6.0±0.2, 9.4±0.2 and 25.2±0.2 degrees two-theta.

11. The crystalline form of claim 1, which exhibits a DSC (differential scanning calorimetry) thermogram substantially similar to FIG. 4, FIG. 5, or FIG. 13.

12. The crystalline from of claim 1, which exhibits a DSC thermogram comprising an exotherm peak (max) between about 200° C. to about 220° C.

13. (canceled)

14. The crystalline form of claim 12, wherein the DSC thermogram further comprises at least two endotherm peaks between about 60° C. to about 180° C.

15. The crystalline form of claim 12, wherein the DSC thermogram further comprises an endotherm peak (max) between about 105° C. to about 130° C.

16. The crystalline form of claim 12, wherein the DSC thermogram further comprises an endotherm peak (max) between about 140° C. to about 170° C.

17. The crystalline from of claim 1, which exhibits a TGA (thermogravimetric analysis) thermogram substantially similar to FIG. 6 or FIG. 12.

18. The crystalline form of claim 1, wherein the crystalline form is isolated.

19.-20. (canceled)

21. A pharmaceutical composition comprising a crystalline form of claim 1 and a pharmaceutically acceptable carrier or excipient.

22.-26. (canceled)

27. A pharmaceutical composition comprising Compound I or a pharmaceutically acceptable salt or a solvate thereof, propylene glycol (PG) and macrogol (15)-hydroxystearate.

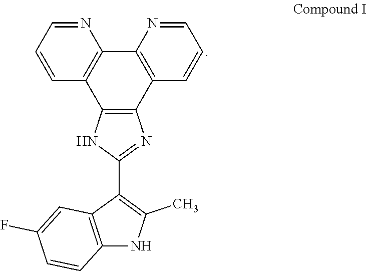 [SMILES: Cc1[nH]c2ccc(F)cc2c1-c1nc2c3cccnc3c3ncccc3c2[nH]1]

28. The pharmaceutical composition of claim 27, wherein the Compound I is Compound I free base tetrahydrate.

29. The pharmaceutical composition of claim 28, wherein the Compound I free base tetrahydrate is the crystalline form of claim 1.

30.-31. (canceled)

32. The pharmaceutical composition of claim 27, wherein the composition is a solution.

33.-34. (canceled)

35. The pharmaceutical composition of claim 27, wherein:
(a) the propylene glycol is present in about 60% to about 80% by volume;
(b) the macrogol (15)-hydroxystearate is present in about 15% to about 30% by volume; and
(c) the water is present in about 3% to about 12% by volume.

36. The pharmaceutical composition of claim 27, wherein:
(a) the propylene glycol is present in about 70% by volume;
(b) the macrogol (15)-hydroxystearate is present in about 23% by volume; and
(c) the water is present in about 7% by volume.

37. The pharmaceutical composition of claim 27, wherein the composition is substantially free of polyethylene glycol.

38. The pharmaceutical composition of claim 27, wherein the composition is diluted in IV fluid selected from sterile water, dextrose in water, glucose in water, invert sugar in water, saline solution in water (NaCl), sodium bicarbonate solution in water, sodium lactate solution in water, lactated Ringer's solution, or combinations thereof.

39. The pharmaceutical composition of claim 27, wherein the composition is diluted in IV fluid selected from 5% dextrose in water, 10% dextrose in water, lactated Ringer's solution, saline solution in water, or combinations thereof.

40. The pharmaceutical composition of claim 38, wherein the Compound I or a salt or a solvate thereof stays in solution for at least about 120 minutes.

41. (canceled)

42. A method of treating cancer, comprising administering the crystalline form of claim 1 to a subject.

43-49. (canceled)

50. A method of treating cancer, comprising administering the pharmaceutical composition of claim 47 to a subject.

51.-70. (canceled)

71. Compound I free base tetrahydrate. 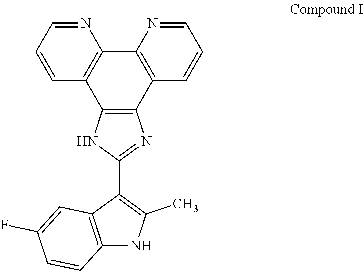 [SMILES: Cc1[nH]c2ccc(F)cc2c1-c1nc2c3cccnc3c3ncccc3c2[nH]1]
